(19) United States
(12) Patent Application Publication
(10) Pub. No.: US 2025/0201406 A1
(43) Pub. Date: Jun. 19, 2025

(54) METHOD AND BIOMARKER FOR PREDICTING SEVERITY OF COVID-19 DISEASE AND METHOD FOR THERAPEUTIC INTERVENTION IN COVID-19 DISEASE

(71) Applicant: University of Central Florida Research Foundation, Inc., Orlando, FL (US)

(72) Inventor: Hung Nguyen, Orlando, FL (US)

(73) Assignee: University of Central Florida Research Foundation, Inc., Orlando, FL (US)

(21) Appl. No.: 18/833,955

(22) PCT Filed: Jan. 31, 2023
(86) PCT No.: PCT/US2023/012002

§ 371 Date: Jul. 29, 2024

Related U.S. Application Data
(60) Provisional application No. 63/304,876, filed on Jan. 31, 2022.

(51) Int. Cl. G16H 50/20; A61K 31/517; A61K 45/06; A61P 31/14; C12N 5/00; C12N 5/0783; G01N 15/10; G01N 15/14; G16B 25/10; G16B 40/30; G16H 50/70

(52) U.S. Cl. CPC G16H 50/20; A61K 31/517; A61K 45/06; A61P 31/14; C12N 5/0018; C12N 5/0636; G01N 15/14; G16B 25/10; G16B 40/30; G16H 50/70; C12N 2501/999; G01N 2015/1006

(57) ABSTRACT

Method for predicting severity of COVID-19 disease resulting from infection with SARS-COV-2 (severe acute respiratory syndrome coronavirus 2). The method uses a high dimensional approach to construct a comprehensive metabolic landscape of immune cells participating in the anti-viral response against SARS-CoV-2. Also encompassed within the invention are novel immune cell subsets exhibiting metabolic dysfunction that could serve as predictive biomarkers for COVID-19 severity or as targets for therapeutic interventions in COVID-19 disease.

### FIELD OF THE INVENTION

The invention is encompassed within the field of immunometabolism and generally relates to analysis of changes in the cellular metabolism of immune cells that can alter their function, particularly relates to analysis of the immunometabolic profile of immune cells in COVID-19 disease resulting from infection with SARS-COV-2 (severe acute respiratory syndrome coronavirus 2), and most particularly relates to using the immunometabolic profile of immune cells (in COVID-19 disease) to identify potential biomarkers for predicting severity of COVID-19 disease and to identify potential therapeutic targets for intervention in COVID-19 disease.

### BACKGROUND

According to the World Health Organization (WHO), over 3.5 million individuals across the world have perished due to the COVID-19 pandemic as of May 2021.¹ Although multiple SARS-CoV-2 vaccines have received emergency use authorization from several countries, global herd immunity gained by vaccinations may not be reached until late 2021 or early 2022.² Additionally, the mutant strains, which can partially evade immunity induced by currently available vaccines, as well as display significantly increased rates of infection, are rapidly increasing in prevalence.³ Therefore, novel therapeutics to tackle SARS-CoV-2 are urgently needed.

Metabolic syndrome and disorders have been recognized as risk factors for severe COVID-19 pathogenesis.^{4,5} The growing body of evidence suggests that individuals with pre-existing metabolic comorbidities are at far higher risks of suffering severe complications from COVID-19.^{6,7} However, there is a lack of understanding about the metabolism of immune cells in the microenvironment of injured organs, such as the lungs, during SARS-CoV-2 infection. Most studies so far have been performed on patients' peripheral blood mononuclear cells (PBMCs).^{4,5,8} Because the metabolic characteristics of the target organ and circulation are different,⁹ knowledge of the immunometabolic landscape in SARS-CoV-2 targeted organs will be essential for generating safe and effective treatments for COVID-19. Additionally, there have not been any metabolic biomarkers, used stand-alone or in combination, that specifically predict the prognosis of COVID-19 patients. Identifying prognostic metabolic biomarkers may provide the urgently needed essential knowledge for clinicians to assess potential for disease severity and triage care accordingly if needed.

### SUMMARY

Metabolic diseases increase the risk of severe COVID-19 symptoms for reasons yet to be fully elucidated. Cellular metabolic dysregulation accompanying COVID-19 infection is a key determinant of disease progression. High-dimensional immune profiling of COVID-19 peripheral blood mononuclear cells (PBMCs), in combination with single cell transcriptomic analysis of COVID-19 bronchoalveolar lavage fluid (BALF) to paint the landscape of immune cells during severe COVID-19 infection, was performed. It was found that hypoxia, a cardinal feature of acute respiratory distress syndrome (ARDS), elicits metabolic reprogramming in immune cells including CD8 T cells, memory NK, NKT, and epithelial cells that increases reliance on anaerobic glycolysis to meet bioenergetic demands. In response to an oxygen and nutrient-deprived environment, CD8 and NKT cells display increased mitophagy and senescence. This metabolic reprogramming induces cellular dysfunction and lymphocytopenia, thereby hampering the host immunological response and derailing anti-viral immunity. Furthermore, direct injury to ciliated epithelial cells by SARS-CoV-2 elevates HLA class 1 machinery and proinflammatory cytokine secretion. Augmented antigen stimulation by epithelial cells, coupled with metabolic dysregulation, likely compromise the effector function and memory cell differentiation of CD8 and NK cells. Multiple distinct, highly resolved CD8 and NK cell subsets that are phenotypically glycolytic and display mitochondrial dysfunction were identified, which may potentially be used as predictive biomarkers for COVID-19 severity. Importantly, lymphocytes from patients infected with the B.1.1.7 UK variant are more glycolytic and functionally exhausted than non-variant. The current studies expand knowledge of the pivotal relationship between cellular metabolism and immune response in COVID-19, and unveils a novel approach for the prediction, treatment and triaging of COVID-19 infected patients.

As noted above, cellular metabolic dysregulation is a consequence of COVID-19 infection that is a key determinant of disease severity. However, how metabolic perturbations influence immune cell function during SARS-CoV-2 infection remains unclear. The instant inventor, using a combination of high-dimensional flow cytometry, cutting-edge single-cell metabolomics in Experiment 2 described below, and re-analysis of single-cell transcriptomic data, demonstrates a global hypoxia-linked metabolic switch from fatty acid oxidation and mitochondrial respiration towards anaerobic, glucose-dependent metabolism in CD8+Tc, NKT, and epithelial cells (ECs). Consequently, it was found that a significant dysregulation in immunometabolism was tied to increased cellular exhaustion, attenuated effector function, and impaired memory cell differentiation. Pharmacological inhibition of mitophagy with Mdivi-1 reduced excessive glucose metabolism, resulting in enhanced generation of SARS-CoV-2-specific CD8′Tc, increased cytokine secretion capacity, and augmented memory cell proliferation. Taken together, this study, described herein in Experiment Two, provides novel cellular and molecular mechanisms underlying the effect of SARS-CoV-2 infection on host immune cell metabolism and highlights immunometabolism as a promising therapeutic target for SARS-CoV-2 treatment.

In a most basic aspect, the invention provides a new treatment modality for COVID-19 disease. COVID-19, the disease caused by infection with SARS-CoV-2 (severe acute respiratory syndrome coronavirus 2), is referred to interchangeably herein as “coronavirus disease 2019”, “COVID-19”, and “COVID-19 disease.”

In another basic aspect, the invention provides a new approach for the prediction of severity, treatment of, and triaging of COVID-19 infected patients.

Aspects of the invention are defined graphically in FIG. 14. Attack of lung epithelial cells by SARS-CoV-2 causes pulmonary damage leading to a hypoxic insult, which in turn increases anaerobic glycolytic metabolism. Concomitant changes of metabolic processes with increased MHC class I expression in lung epithelial cells (ECs) cause immune cell exhaustion (CD8 T and NK cells) which is evident by characteristics of mitochondrial dysfunction; oxidative stress, senescence, glycolysis and inflammatory phenotype. An accompanying insidious effect is interference with immune memory cell formation, which increases the vulnerability of the host to reinfection by SARS-CoV-2. Therefore, protecting immune cell function in the face of a SARS-CoV-2 challenge by preserving normal immunometabolism is a promising approach to treat COVID-19 patients and improve clinical outcome.

In an aspect, the invention provides a method for predicting severity of COVID-19 disease in a patient having or suspected of having COVID-19 disease. The patient is preferably, but not limited to, a human. The method includes: obtaining a sample of bronchoalveolar lavage fluid (BALF) from the patient; performing a single cell transcriptomic analysis of the BALF obtained from the patient; obtaining a sample of bronchoalveolar lavage fluid (BALF) from a healthy subject; performing a single cell transcriptomic analysis of the BALF obtained from the healthy subject; comparing the single cell transcriptomic analysis obtained from the patient to the single cell transcriptomic analysis obtained from the healthy subject and identifying differences from the comparison which indicate biomarkers of the severity of COVID-19 disease; using the biomarkers indicated to predict the severity of COVID-19 disease in the patient; obtaining a blood sample from the patient to isolate peripheral blood mononuclear cells (PBMCs) from the blood sample; performing high-dimensional immune profiling of the PBMCs obtained from the patient to identify and characterize the populations of lymphocytes contained therein; obtaining a blood sample from the healthy subject to isolate the PBMCs from the blood sample; performing high-dimensional immune profiling of the PBMCs obtained from the healthy subject to identify and characterize the populations of lymphocytes contained therein; comparing the populations of lymphocytes identified from the patient to the populations of lymphocytes identified from the healthy subject and determining differences from the comparison which indicate biomarkers of the severity of COVID-19 disease; and using the biomarkers indicated to predict the severity of COVID-19 disease in the patient.

The “healthy subject” or “healthy patient” does not have nor is suspected of having COVID-19 and is preferably, but not limited to, a human.

In carrying out the described methods, biological samples, such as, but not limited to, BALF and PBMCs, may be obtained from patients having or suspected of having COVID-19 disease, healthy patients, and patients having non-COVID respiratory infections. The samples may be obtained from a single patient type or from any combination of patient types.

In an aspect of the method, performing the single cell transcriptomic analysis of the BALF includes, but is not limited to, quality control and batch effect correction; unsupervised clustering and non-linear dimensionality reduction for high-dimensional immunophenotyping; differential expression and abundance analysis; and nonparametric correlation analysis.

In an aspect of the method, obtaining the blood sample includes, but is not limited to, isolating the PBMCs from the whole blood using density dependent centrifugation.

In an aspect of the method, the high-dimensional immune profiling is carried out using flow cytometry.

In an aspect of the method, subsets of immune cells are identified as biomarkers useful for predicting severity of COVID-19 in the patient. Examples include, but are not limited to, GLUT1⁺VDAC⁺ CD8 cells, GLUT1⁺VDAC⁺ CD8 NKT cells, and GLUT1⁺CD62L⁺NK cells.

In another aspect of the method further steps may be added to the protocol, such as, but not limited to, carrying out single-cell metabolomics on lymphocytes identified from the population of lymphocytes contained within the PBMCs obtained from the patient and on lymphocytes identified from the population of lymphocytes contained within the PBMCs obtained from the healthy subject and carrying out scRNA-SEQ reanalysis. The identified lymphocytes can be, but are not limited to, at least one of CD8 cells, such as, but not limited to CD8 memory cells, and Natural Killer T cells (NKTs).

In another aspect, carrying out single-cell metabolomics includes carrying out a SCENITH metabolomics assay which can include carrying out a glucose uptake assay.

Another aspect of the invention includes a biomarker for predicting severity of COVID-19 disease in a patient having COVID-19 disease including a metabolic profile of a CD8 cell, such as, but not limited to, a CD8 memory cell or CD8 cytotoxic cell.

Another aspect of the invention provides a method for treatment of COVID-19. Treatment can be, but is not limited to, modulation of cellular processes to preserve healthy immunometabolism in the lungs during COVID-19 disease. Examples are, but not limited to, inhibition of glycolysis, inhibition of mitophagy, and/or inhibition of lymphocytopenia. A variety of modulating compounds can be used. Examples include, but are not limited to, inhibitors of lactate dehydrogenase A (LDHA) such as FX11(FX11 [3-dihydroxy-6-methyl-7-(phenylmethyl)-4-propylnaphthalene-1-carboxylic acid]) and glucocorticoids (dexamethasone) and inhibitors of mitophagy such as Mdivi-.

Another aspect of the invention includes providing and administrating a composition including Mdivi-1 to both cells and patients. This administration can have multiple aims, such as, but not limited to, inhibition of mitophagy in cells (such as, but not limited to, lymphocytes and epithelial cells) isolated from bronchoalveolar lavage fluid (BALF); restoration of lymphocyte function in lymphocytes (such as, but not limited to, CD8⁺T_{C} and CD8⁺T_{M}) isolated from a patient having COVID-19 disease; restoration of proliferation, activation, and memory formation of CD8⁺T_{C} and CD8⁺T_{M} isolated from a patient having COVID-19 disease; inhibition of mitophagy in lymphocytes (such as, but not limited to, CD8⁺T_{C} and CD8⁺T_{M}) of a patient having or suspected of having COVID-19 disease; treatment of COVID-19 disease in a patient having or suspected of having COVID-19 disease; and inhibition of viral replication of SARS-CoV-2 in cells of a patient having COVID-19 disease.

In a further aspect, treatment of COVID-19 disease can include the administration of other drugs known as beneficial in treating COVID-19 with Mdivi-1. Examples of such drugs, include, but are not limited to cyclophilin, a cholesterol-lowering drug, a glucose-metabolism reducing drug, and an antioxidant drug.

In another aspect, the invention includes a pharmaceutical composition for treatment of COVID-19 disease comprising a therapeutically effective dosage of Mdivi-1 in at least one pharmaceutical carrier. The “pharmaceutical carrier” can be any inactive and non-toxic substance useful for preparation of medication. The phrase “therapeutically effective dosage” or “therapeutically effective amount” refers to the amount of a composition required to achieve the desired function; for example, treatment of COVID-19 disease.

Other objectives and advantages of this invention will become apparent from the following description, wherein are set forth, by way of example, certain embodiments of this invention.

### Abbreviations

COVID-19, coronavirus disease 2019, COVID-19 disease
SARS-CoV-2, severe acute respiratory system coronavirus 2 causes COVID-19
ARDS, acute respiratory distress syndrome
CD8⁺Tc, CD8 T lymphocytes
BALF, bronchoalveolar lavage fluid
PMBC, peripheral blood mononuclear cells
FAO, fatty acid oxidation
CRS, cytokine released syndrome
CTL, cytotoxic T lymphocyte
SASP, senescence-associated secretory phenotype
EC, epithelial cells
Trm, T resident memory
ARDS, acute respiratory disease syndrome
p38 MAPK, p38 mitogen-activated protein kinase
LDHA, lactate dehydrogenase
PCA, principal component analysis
UMAP, universal manifold approximation and projection
CCA, canonical correlation analysis
ICU, intensive care unit

### BRIEF DESCRIPTION OF THE DRAWINGS

A more complete understanding of the present invention may be obtained by references to the data shown in the accompanying drawings when considered in conjunction with the subsequent detailed description. Any embodiments illustrated in the drawings are intended only to exemplify the invention and should not be construed as limiting the invention to the illustrated embodiments.

FIGS. 1A-L present data showing that high-dimensional immunophenotyping revealed a highly resolved single cell landscape of COVID-19 brochoalveolar lavage fluid (BALF) and peripheral blood mononuclear cells (PBMCs).

FIGS. 2A-P present data showing the metabolic profile of cytotoxic T lymphocytes (CTLs) in COVID-19, with FIGS. 2H-K evidencing that metabolic dysfunction is evident in CD8⁺Tc (cytotoxic T lymphocytes) from severe COVID-19 patients and FIGS. 2L-P evidencing that CD8⁺Tc are functionally exhausted in severe COVID-19 patients.

FIGS. 3A-L present data showing the metabolic profile of CD8 memory cells in COVID-19, with FIGS. 3A-F evidencing metabolic reprogramming in BALF CD8⁺T_{M} during SARS-CoV-2 infection, FIGS. 3G-K evidencing that metabolic reprogramming towards anaerobic glycolysis upon mitochondrial dysfunction occurs in CD8⁺T_{M} during SARS-CoV-2 infection, and FIGS. 3L-O evidencing that metabolic dysregulation triggers functional impairment in CD8⁺T_{M}.

FIGS. 4A-D present data showing that COVID-19 induced metabolic dysfunction impairs memory formation and cellular differentiation of CD8 T-cells.

FIGS. 5A-J present data showing metabolic disorder of epithelial cells in COVID-19.

FIGS. 6A-G present data showing that metabolic disorder impairs immune surveillance and inflammatory response of ECs.

FIGS. 7A-M present data showing aberrant metabolism of natural killer T-cells (NKTs) and CD62L+ cells in COVID-19 patients.

FIGS. 8A-D present data showing differential metabolism of memory cells in B.1.1.7 variant infected patients.

FIGS. 9A-B show heatmaps displaying canonical gene expression of genes used to annotate unsupervised clusters for all 66,452 cells after sample integration.

FIG. 10 shows a heatmap displaying canonical gene expression of genes used to annotate unsupervised clusters after T cell reintegration.

FIGS. 11A-G show heatmaps displaying canonical marker expression of phenotypic markers.

FIGS. 12A-D show heatmaps displaying expression of key differentially expressed metabolic genes.

FIGS. 13A-B show UMAP projection of 2603 CD4 cells.

FIG. 14 is a graphical summary of cellular metabolic dysregulation accompanying coronavirus disease 2019 (COVID-19).

FIGS. 15A-F show data evidencing that mitophagy inhibition restores CD8⁺Tc and SARS-CoV-2 specific CD8⁺Tc function.

FIG. 16 shows the representative flow cytometry gating strategy.

FIGS. 17A-C show data evidencing unsupervised clustering for CD8⁺Tc and NK of patient PBMCs.

FIGS. 18A-C show data evidencing an association between prior comorbidities and CD8⁺Tc metabolic dysfunction.

FIGS. 19A-G show data evidencing abnormal metabolic phenotype of BALF effector CD8⁺Tc from severe COVID-19 patients.

### DETAILED DESCRIPTION

For the purpose of promoting an understanding of the principles of the invention, the research will now be presented and discussed. Reference will be made to embodiments illustrated herein and specific language will be used to describe the same. It will nevertheless be understood that no limitation of the scope of the invention is thereby intended. Any alterations and further modification in the described assays, biomarkers, methods, procedures, therapeutics, and/or compositions along with any further application of the principles of the invention as described herein, are contemplated as would normally occur to one skilled in the art to which the invention relates.

Additionally, the term “or” is intended to mean an inclusive “or” rather than an exclusive “or.” That is, unless specified otherwise or clear from context, “X employs A or B” is intended to mean any of the natural inclusive permutations. That is if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

Unless explicitly stated otherwise, each numerical value and range should be interpreted as being approximate as if the word “about” or “approximately” preceded the value of the value or range.

It should be understood that the steps of the methods set forth herein are not necessarily required to be performed in the order described, and the order of the steps of such methods should be understood to be merely exemplary. Likewise, additional steps may be included in such methods, and certain steps may be omitted or combined in methods consistent with various embodiments of the present device.

Although the elements in the following method claims, if any, are recited in a particular sequence with corresponding labeling unless the claim recitations otherwise imply a particular sequence for implementing some or all of those elements, those elements are not necessarily intended to be limited to being implemented in that particular sequence.

### INTRODUCTION

Immunological dysfunction is well-recognized in COVID-19 patients.^{10,11} A hallmark characteristic of severe SARS-CoV-2 infection is lymphocytopenia,¹ which is characterized by reduced abundance and compromised function of lymphocytes, leading to an impaired SARS-CoV-2-specific T cell antiviral response.^{12,13} However, the immunological mechanisms responsible for T cell dysfunction remain elusive. Given that cellular metabolism and bioenergetics dictate T cell fate and function,¹⁴ the instant inventor hypothesized that cellular metabolism plays a critical role in dictating T cell responses during severe COVID-19.

The lung is the primary target organ of SARS-CoV-2, as the spike protein directly binds to ACE2 (angiotensin converting enzyme 2) receptors expressed on the surface of lung epithelial cells.¹⁵ As a result, acute respiratory distress syndrome (ARDS) often occurs in severe COVID-19, resulting in decreased blood oxygen saturation levels (hypoxia), as well as increased serum lactate dehydrogenase (LDHA) levels.^{11,16,17} Both downstream hypoxia signaling and hyperlactatemia (increased blood lactate level) have been associated with proinflammatory cytokine syndrome and lymphocyte dysfunction.^{12,18} However, it is not understood whether/how hypoxia in COVID-19 ARDS patients affects the metabolic phenotype of lymphocytes. Thus, there is an urgent need to identify distinct cell subsets with metabolic dysfunction that are responsible for immunological dysfunction in the lungs during severe COVID-19.

SARS-CoV-2 viruses are constantly changing through mutation. There have been multiple variants of the virus including United Kingdom (UK) variant (a), the South Africa variant (0), and the India variant (A) have been cumulatively reported.¹⁹ These variants are more contagious, increase risk of death, and also have a significant impact on the effectiveness of monoclonal antibody medications and COVID-19 vaccine or previous infection-inducing antibodies¹⁹. However, it is unknown whether immunometabolism is related to different pathogenesis during SARS-CoV-2 variant infection.

In the study/research described herein, a publicly available single cell sequencing dataset from the bronchoalveolar lavage fluid (BALF) of COVID-19 patients was used to produce a transcriptional gene-level landscape of metabolic activity in multiple cell subsets at a single-cell resolution in the complex COVID-19 lung microenvironment. Next, a high-dimensional flow cytometry of hospitalized COVID-19 patient PBMCs to validate the gene-expression results at a proteomic level was performed. Flow cytometry was performed specifically on freshly isolated and unstimulated cells. It was found that hypoxia and anaerobic glycolysis are the driving factors responsible for lymphocytopenia in COVID-19 patients. These factors not only attenuate T-cell immunity, but also suppress their capacity to transit into memory phenotype, affecting the generation of effective immunity against COVID-19. It is demonstrated for the first time that memory lymphocytes in patients infected with the UK variant B.1.1.7 display a distinguishable metabolic profile, which is characterized by further increased aerobic glycolysis and cellular exhaustion as compared to non-variant. Accordingly, this study provides a rationale for the relationship between metabolic disorders and COVID-19 severity, as well as unveiling multiple promising metabolic pathways and targets for the control of COVID-19 severity and the enhancement of COVID-19 vaccine efficacy. The findings in this study provide a single-cell metabolic landscape of COVID-19, highlighting the metabolic plasticity and heterogeneity of immune cells during the COVID-19 infection cascade. The study also proves that glycolytic and mitochondrial reprogramming highly regulate the metabolic features of immune cell subtypes. Many of these cell subsets can be used as biomarkers for COVID-19 severity. Therefore, the study/research described herein has tremendous implications for advancement in COVID-19 severity assessment, treatment, and therapy.

### Experiment One

### Methodology

### Sample Acquisition

Blood samples from healthy donors were ordered from Research Blood Company. Blood samples from hospitalized COVID-19 patients were collected from the AdventHealth hospital under protocols IRB #1668907 and #1590483 approved by AdventHealth IRB committee. Strict confidentiality was maintained for all patients according to HIPAA confidentiality requirements. COVID-19 was confirmed by PCR test at AdventHealth. Blood was used for human PBMC, plasma, and serum isolation.

### PBMC Isolation

PBMCs were isolated by density-gradient centrifugation using Ficoll. Briefly, blood specimens were centrifuged at 700 G for 7 min at RT for serum collection. The pellets were resuspended in phosphate buffer saline (PBS). Cell suspensions were carefully overlaid on the top of 4 mL Ficoll in 15 mL conical tube, followed by centrifugation at 700 G at RT for 25 min without break. PBMCs were collected from interphase between plasma and Ficoll layers. Cells were washed twice with PBS to remove Ficoll residue. All the procedures were approved at BSL2⁺ level by UCF Environmental Health and Safety.

### Antibody Staining and Flow Cytometry

About 5×10⁵ cells from each sample were used for flow cytometry staining. See Table 1 below for antibody information. PBMCs were first stained with live/dead in PBS for 15 min, washed with FACS buffer, and stained with surface markers in FACS buffer at 4° C. for 30 min. Following incubation, PBMCs were washed (FACS buffer, 200 μL), stained with secondary antibody mix for another 15 min, and then washed again with FACS buffer. Samples were fixed and permeabilized with Fixation/Permeabilization buffer (15 min) and washed with FACS buffer. PBMCs were then stained with the intracellular antibody staining mix at 37° C. for 45 min. Samples were washed once with Fixation/Permeabilization buffer before being resuspended in FACS buffer for flowcytometric analysis.

TABLE 1

| Reagent/Resource | Source | Identifier |
|---|---|---|
| Anti-human LAG-3 PE/Dazzle | BioLegend | Clone- |
| | | 11C3C65/369331 |
| Anti-human CD62L PerCP/Cy 5.5 | BioLegend | Clone DREG- |
| | | 56/304823 |
| Anti-human CCR7 BV510 | BioLegend | Clone |
| | | G043H7/353231 |
| Anti-human/mouse Granzyme B PE/Cy7 | BioLegend | Clone |
| | | QA16A02/372213 |
| Anti-human CD56 BV650 | BioLegend | Clone |
| | | HCD56/318343 |
| Anti-human GLUT-1 AF700 | R&D Systems | Clone 202915/ |
| | | FAB1418N |
| Anti-VDAC | Alomone Labs | avc-001-50 mcl |
| CD8 APC/Cy7 | | |
| Anti-human/mouse HIF-1 APC | R&D Systems | Clone 248182/ |
| | | IC1935A |
| H2DCFDA | ThermoFisher | D399 |
| Milk Powder | AmericanBio | AB10109-01000 |
| PBS | Gibco | 10010-023 |
| Water For Injection | Gibco | A1287301 |
| Tween 20 | Fisher | BP337-500 |
| | Bioreagents | |
| Anti-Human IgG (Fab specific)-Peroxidase antibody | Sigma | A0293 |
| Hydrochloric Acid | Fisher Scientific | S25856 |
| SIGMAFAST ™ OPD | Sigma-Aldrich | P9187 |
| Spike Glycoprotein Receptor Binding Domain (RBD) | Bei Resources | NR-54004 |
| from SARS-Related Coronavirus 2, United Kingdom | | |
| Variant | | |
| Spike Glycoprotein Receptor Binding Domain (RBD) | Bei Resources | NR-52366 |
| from SARS-Related Coronavirus 2, Wuhan-Hu-1 | | |
| with C-Terminal Histidine Tag, | | |

### Spike Confirmatory ELISA

The ELISA setup and protocol for detection of the SARS-CoV-2 spike protein was adapted from previous publication in the online library of the Wiley website. A 96-well flat-bottom immunoplate (Extra Gene, USA) was coated with 100 μl/well of SARS-CoV-2 specific recombinant nucleoprotein (2 μg/mL) in coating buffer PBS and incubated either at 37° C. for an hour or overnight at 4° C. The coating solution and unbound antigen were removed by adding 200 μl per well of 3% non-fat milk prepared in PBS with 0.1% Tween 20. PBST was added to the plates at room temperature for 1 hr as a blocking solution. Serum samples were heat-inactivated at 56° C. for 1 h before use to reduce the risk from any potential residual virus in the serum. Serum (positive and negative) and antibody samples were prepared in 1% non-fat milk prepared in PBST. 100p of test serum was added into each well and incubated at 22° C. for 2 hr at room temperature. Next, a 1:3,000 dilution of anti-human IgG (Fab specific) HRP labeled secondary (Thermo Fisher Scientific) antibody was prepared in 0.1% PBST and 100 μl of this secondary antibody was added to each well for 1 h. Plates were again washed three times with 0.1% PBST. 100 μl SIGMAFAST OPD (o-phenylenediamine dihydrochloride; Sigma-Aldrich) solution was added to each well. Reaction was left to develop for 10 min and stopped by the addition of 50 μl per well of 3 M hydrochloric acid. The optical density at 490 nm (OD490) was measured using a Synergy 4 (BioTek) plate reader. The background value was set at an OD490 of 0.035. True positive samples had a signal higher than the negative control plus 3 standard deviations of the negative controls in at least two consecutive dilutions.

High-dimensional Flow Cytometry Analysis First, the flowCore package in R was used to read in compensated FCS files into the R environment.⁹⁵ Automatic gating functions in openCyto were next used to filter cells for doublets and debris.⁹⁶ Next, an arcsinh transformation with a cofactor of 5 was applied for data normalization. Data from all the samples was then merged into one Catalyst object, upon which downstream analyses were performed.⁹⁷ CytoNorm was next applied to correct for batch effect between samples by aligning peaks of bimodally distributed markers.²⁵ PCA was then run on bulk sample-aggregated data and the top 3 principal components were plotted. FlowSOM clustering was performed on only cell surface markers used for phenotypic identification with the number of expected populations set at 20.²⁶ Clusters were then annotated based upon canonical marker expression. For purposes of UMAP dimensionality reduction, clusters that could not be labelled were discarded before UMAP was performed. Differential abundance of cell-type proportions and differential expression of MFI values were next conducted. For each identified population of interest, FlowSOM was applied again on only the functional state markers with the number of expected populations set at 10. Unsupervised clusters were then annotated by canonical marker expression to define highly resolved cell states for specific populations.

### BALF Data Acquisition

Single cell RNA-seq data from the BALF of 6 severe COVID patients, 3 moderate patients, and 4 healthy donors were used for analysis.²² This study defined moderate and severe COVID-19 patients as those with pneumonia experiencing respiratory distress and hypoxia and with critical condition, requiring ICU care, and having been placed under mechanical ventilation, respectively. Prefiltered expression matrices with UMI counts were downloaded from the GEO Database with accession number GSE145926. Additionally, as suggested by the original study, data from an additional BALF sample derived from a healthy donor from a separate study was used as a reference.⁹⁸ Prefiltered expression matrices with UMI counts were downloaded from the GEO Database with accession number GSE128033 and sample number GSM3660650.

### Data Quality Control and Preprocessing

Quality control and data preprocessing was done using Seurat.^{99,100} First, cells for which more than 10% of reads were mitochondrial transcripts were discarded. Next, cells that had less than 1000 detected transcripts were removed. Cells with less than 200 and greater than 6000 unique genes were also filtered. Filtered data from different 14 patient samples was integrated in Seurat. Individually, data from each sample was log 2 normalized and the top 2000 variable genes were identified using the “vst” method in Seurat. Data from each sample was next scaled and PCA was run with percentage of mitochondrial DNA and number of detected unique genes regressed out. Alignment and batch effect correction was done using reciprocal PCA and canonical correlation analysis (CCA) (in accordance with standard Seurat integrated analysis workflow) on the first 30 dimensions of the data. Next, a shared nearest neighbor graph was constructed and Louvain-based optimization was run to perform unsupervised clustering. UMAP was next run on the first 30 dimensions. Data was next log 2 normalized and scaled in the “RNA” assay for expression analysis, with percentage of mitochondrial DNA and number of detected unique genes regressed out. The top 2000 variable genes were determined by the “vst” method in Seurat. Expression of canonical markers was used to define cell populations. For each identified cell population, SCTransform was done on the “RNA” assay to improve normalization and aid in visualization purposes.

### T Cell Reintegration

T cells were subsetted and split according to samples. Data from healthy control 1 and severe 1 were excluded from analysis due to low T cell count. To further correct for batch effect, T cells were then reintegrated using canonical correlation analysis in Seurat run on the first 30 dimensions. SCTransform was next implemented on the “RNA” assay and stored in a new “SCT” assay to better normalize counts across samples for visualization purposes with percentage of mitochondrial DNA regressed out. Standard log 2 normalization and scaling was then performed on the “RNA” assay. Subpopulations of T-cells were next identified based upon canonical marker expression.

### Trajectory Inference and Pseudo-Temporal Ordering

Monocle 3 was used to construct a trajectory upon UMAP embeddings and order cells in pseudotime.” Analysis was done on both CD8 and CD4 T cells. Seurat wrapper function “asMonocle” was used to create Monocle CellDataSet object from an existing Seurat object. “learn_graph” function was used to construct trajectory mappings onto transferred UMAP embeddings. “order_cells” was used to estimate and order cells in pseudotime. All samples for CD8 and CD4 populations were ordered together and were split by disease state after ordering for differential comparison of pseudotime.

### Metabolic Phenotype Based Clustering

To investigate whether metabolic phenotypes of certain cell populations alone could be used alone as predictive indicators of disease severity, dimensionality reduction at both a single cell and sample-wide resolution was done only on key identified differentially expressed metabolic genes to see if cells/samples would cluster according to disease severity. For sample-wide analysis, principal component analysis was conducted and the first three principal components were visualized. For analysis at single cell resolution, UMAP was done and the first two components were visualized.

### Network Analysis

For construction of gene pathway enrichment network, networkanalyst.ca was used.¹⁰² All statistically significant genes were input along with log fold change values to construct enrichment network. Transcription factor-gene interaction network was also constructed using networkanalyst.ca.¹⁰² Statistically significant genes along with log fold changes vales were input. The “degree” filter was first set to 100 and then the “betweenness” filter was set to 170.

### Downstream Analysis

For heatmap visualizations, scaled SCTransformed values were used and the Complexheatmap package was used to generate visualization.¹⁰² Hierarchical clustering and dendrogram generation were done using default settings of the package. Outliers with extremely high scaled expression values (2) were set to a maximum value of 2 to not distort. For dotplot visualizations, first a Euclidean distance matrix was generated for which hierarchical clustering was then applied. Ggtree was next used for dendrogram construction.¹⁰³ ReactomePA package was used for functional GSEA.¹⁰⁴ All unique detected genes in the cell subset were sorted by log fold change values to create ranked list that was inputted for GSEA analysis. enrichR was used to determine over and under expressed pathways from differential expression analysis (Kuleshov).¹⁰⁵ Corrplot package was used for generation of correlation matrices. Volcano plots were constructed using EnhancedVolcano. Other graphical visualizations were created using ggplot2, ggpubr or plotly. All further downstream analysis was done in base R.

### Statistical Analysis

Differential expression analysis of transcript abundance was assessed using Seurat's implementation of the nonparametric Wilcoxon rank-sum test. Genes were generally defined as statistically significant by Bonferroni adjusted p. values less than 0.05 and log-fold change greater than 0.25. For NKT cells, non-adjusted p. value was used to define differentially expressed genes due to very small sample size.

For comparison of cell-type proportions, nonparametric Wilcoxon rank-sum test was performed, and non-adjusted p values were used to indicate significance. To compare median fluorescent intensity (MFI), nonparametric Wilcoxon rank-sum test was also used to evaluate significance. Additionally, Pearson correlation coefficient was used to indicate strength of measured correlations. Student's t test was used to evaluate significance of measured correlation.

### Experiment One: Results and Detailed Figure Description

### A) Results

### Transcriptional Landscape Analysis of BALF Cells Reveals Dysfunctional Innate and Adaptive Immunity in COVID-19

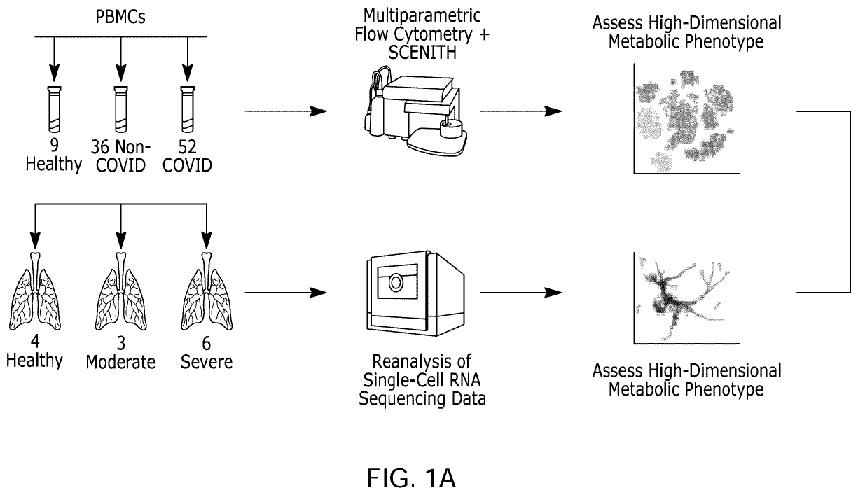

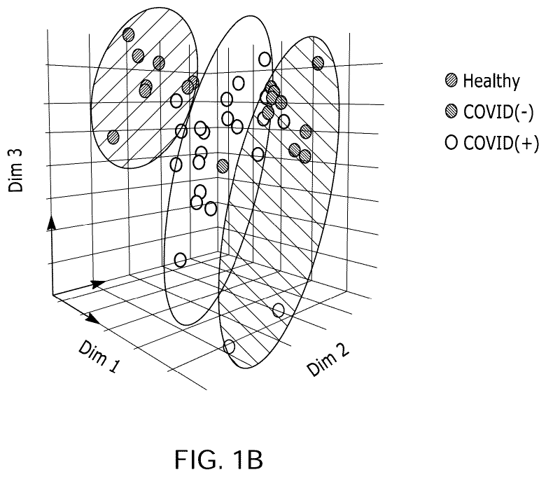

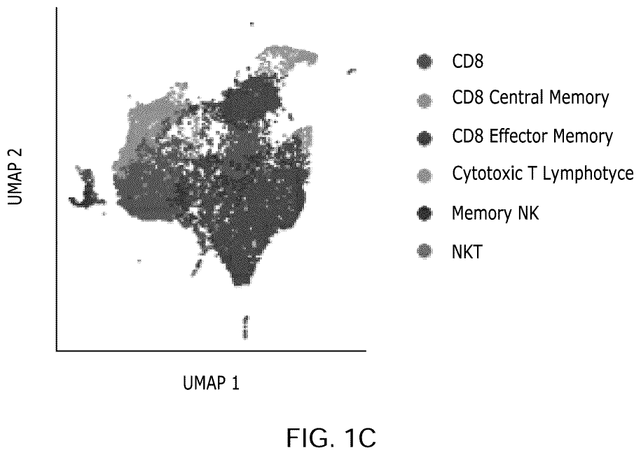

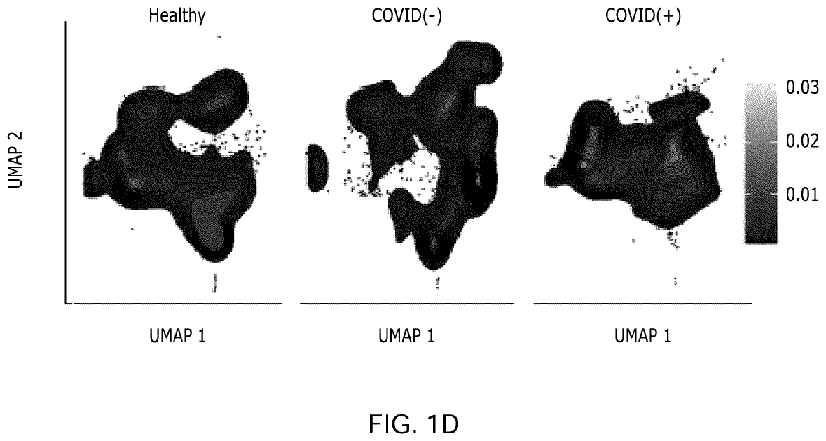

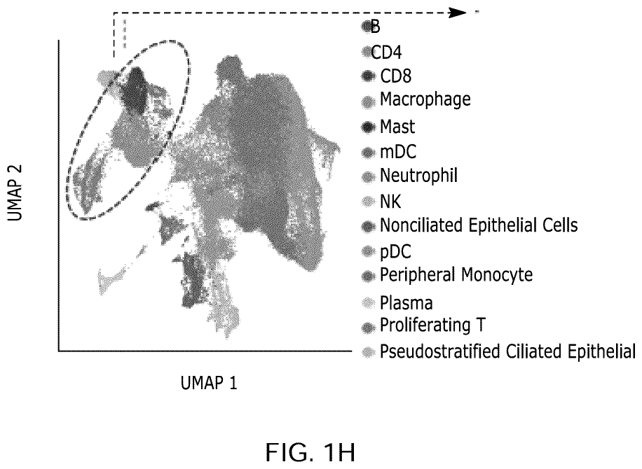

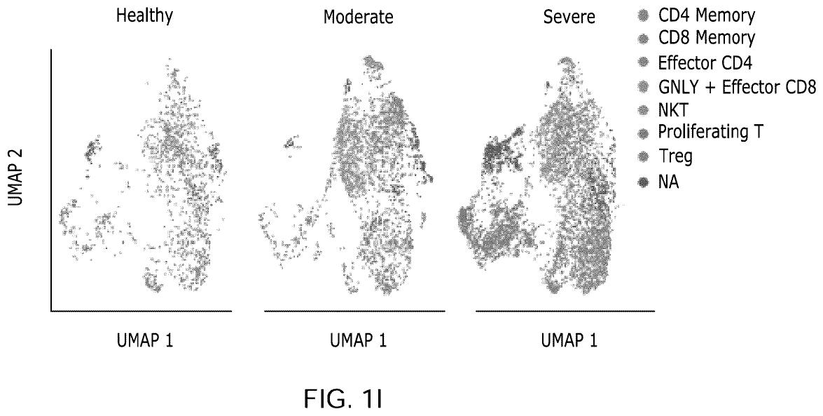

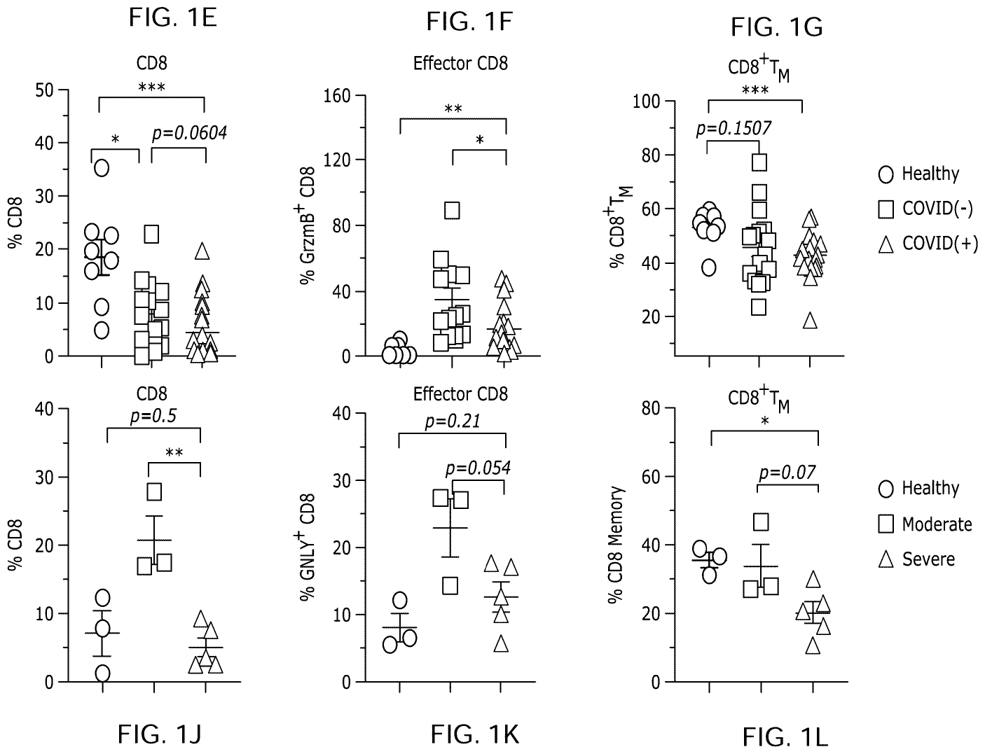

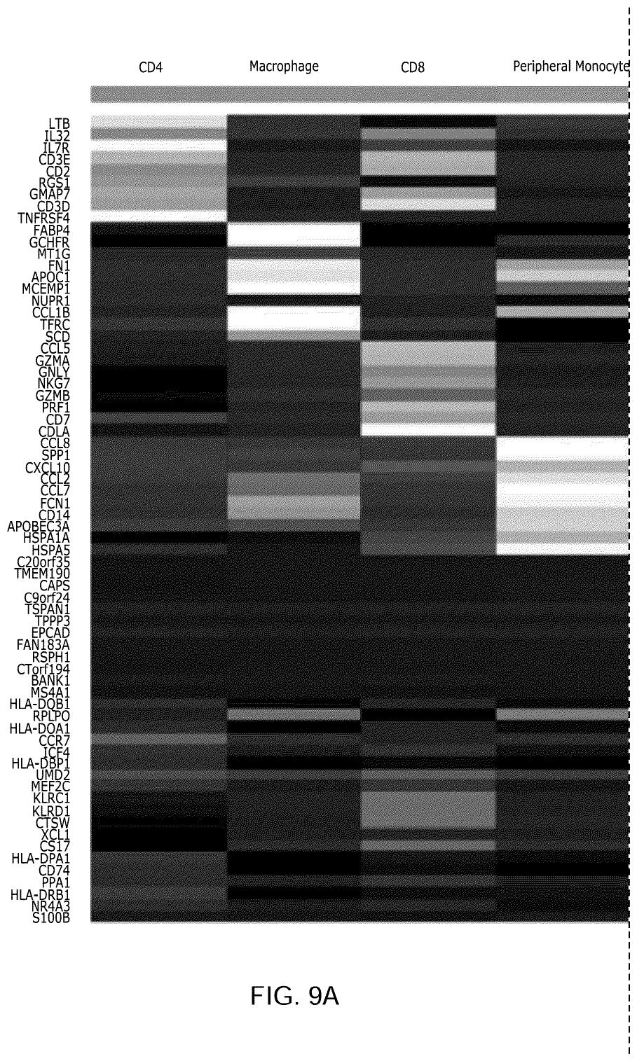

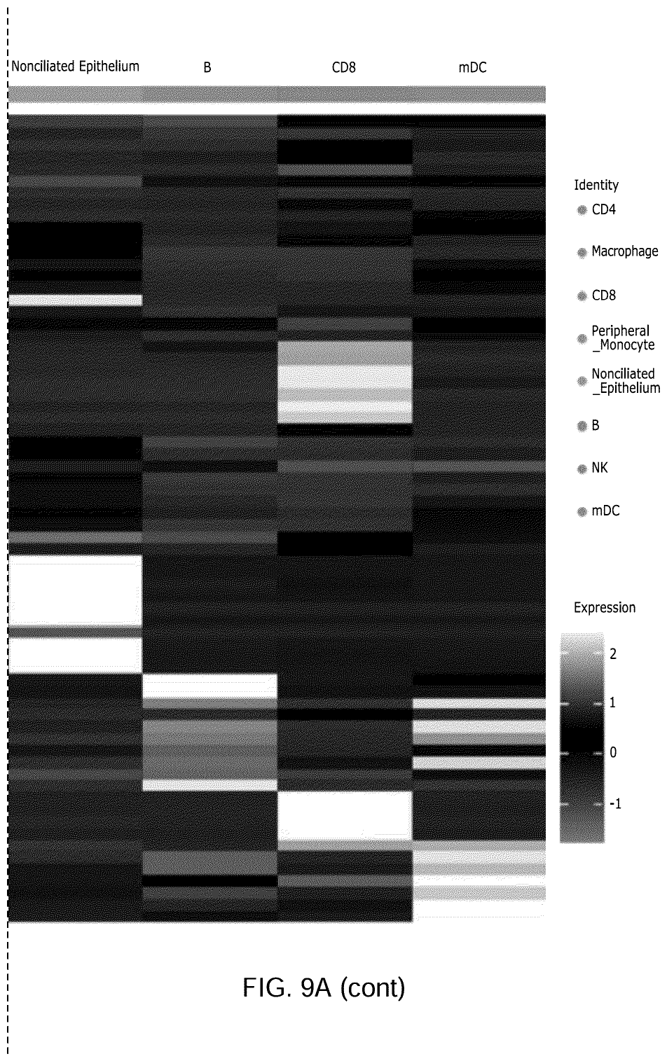

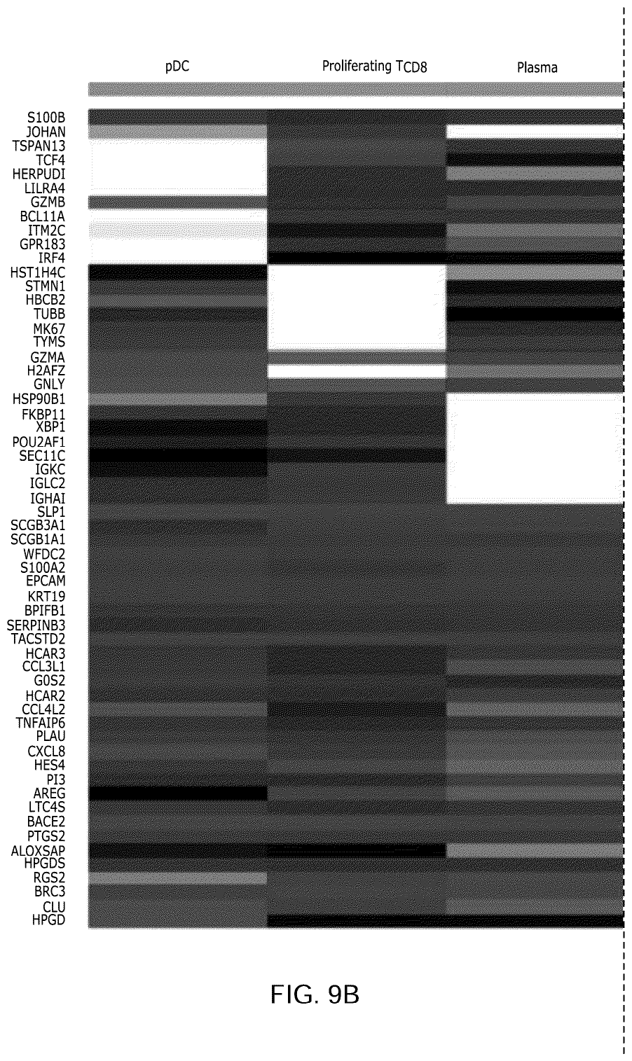

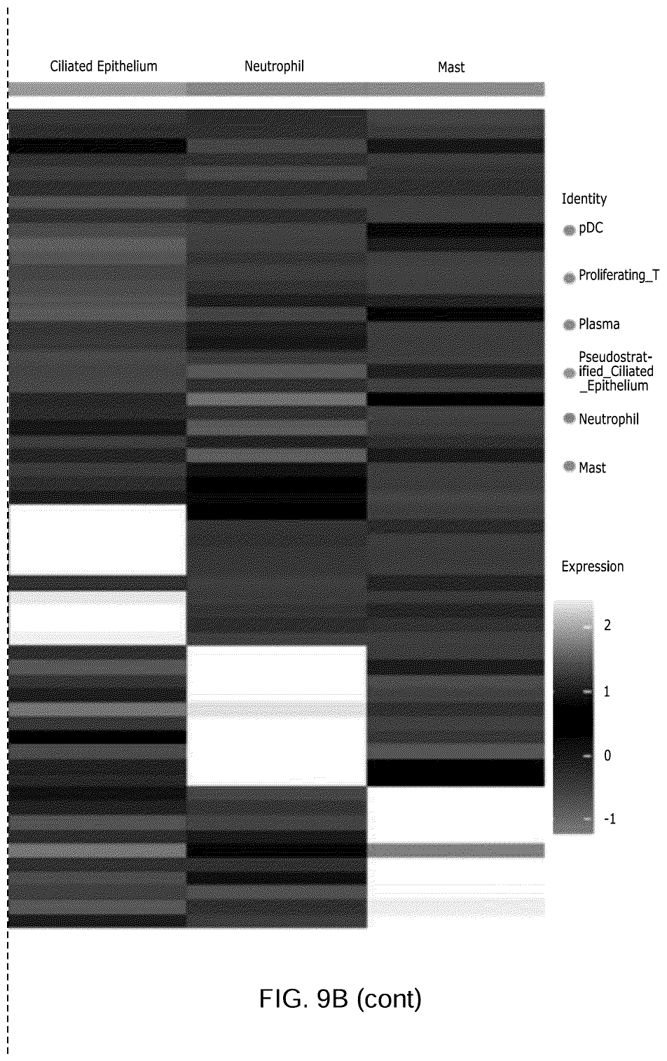

To understand the immunological response to SARS-CoV-2, transcriptomic analysis of publicly available single cell RNA-sequencing data from patients infected with SARS-CoV-2¹⁹ was performed. 66,452 cells derived from BALF samples of 4 healthy, 3 moderately-ill (moderate), and 6 severely ill (severe) patients were retained after initial quality control.²⁰ Cells from each sample were log-normalized and scaled. Anchor-based integration, along with principal component analysis (PCA) and canonical correlation analysis (CCA), were used to combine data and remove batch effect. Universal manifold approximation and projection (UMAP) was performed for dimensionality reduction before Louvain-based optimization to identify unsupervised clusters. The UMAP plot in FIG. 1A demonstrates the annotation of unsupervised clusters into distinct, highly resolved populations for all 66,452 cells from all samples. Specifically, canonical markers used to annotate the unsupervised clusters and define cell subsets were as follows: CD8⁺ T cells (CD3D and CD8A); CD4⁺T cells (CD3D and CD4); proliferating T cells (CD3D and MK167); pseudostratified epithelial cells (EPCAM, CFAP126, and DNAAF1); non-ciliated epithelial cells (EPCAM); plasma cells (IGKC and MS4A1(−)); B cells (MS4A1); neutrophils (HCAR3); NK cells (KLRC1); MAST cells (LTC4S); pDCs (CLEC4C); myeloid dendritic cells (mDCs) (CD1C and CLEC9A); macrophages (CD68 and FABP4); peripheral monocytes (CD68, FCN1, and CD14) (FIGS. 9A-B). Differential abundance analysis of cell subsets was next performed. The proportion of CD8 cells was significantly elevated in moderate compared to severe and healthy patients suggesting a failure of generating robust CD8 response as a major factor driving disease. Lymphocytopenia may arise from innate immune cell dysfunction, such as impaired antigen presenting capacity. Indeed, the frequency of mDCs was remarkably diminished in severe compared to moderate patients (FIGS. 1B-C). Furthermore, monocyte-to-macrophage differentiation was impaired in COVID-19, demonstrated by the inverse correlation of these cell subsets with disease severity (FIG. 1B). A significant influx of neutrophils (PMN) in severe patients was also observed (FIG. 1B). It is well-known that accumulation of PMNs is an important factor contributing to cytokine release syndrome (CRS), a condition that causes lymphocytopenia.²¹ (FIG. 1B).

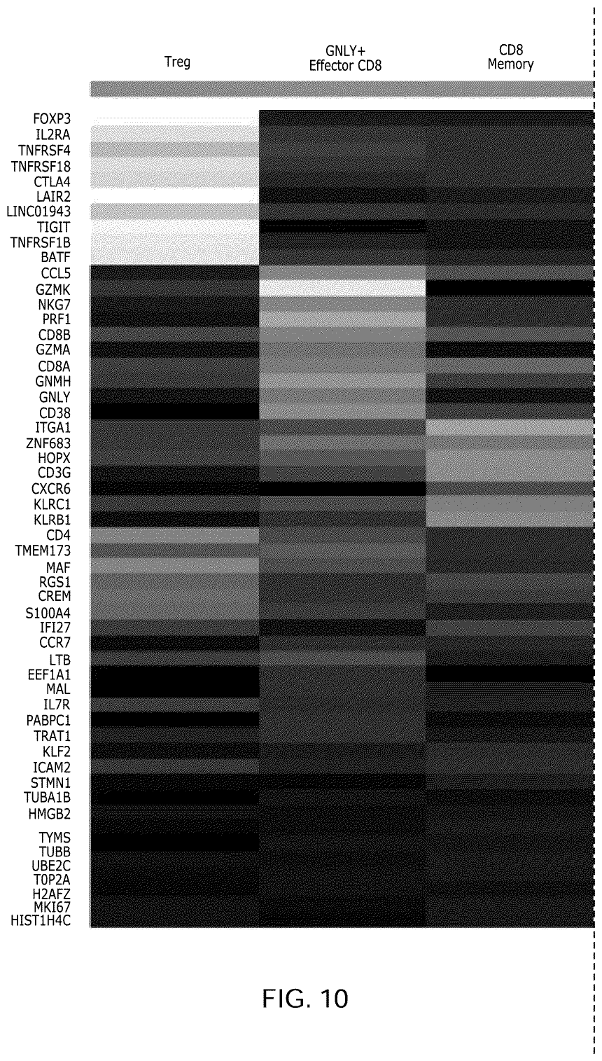

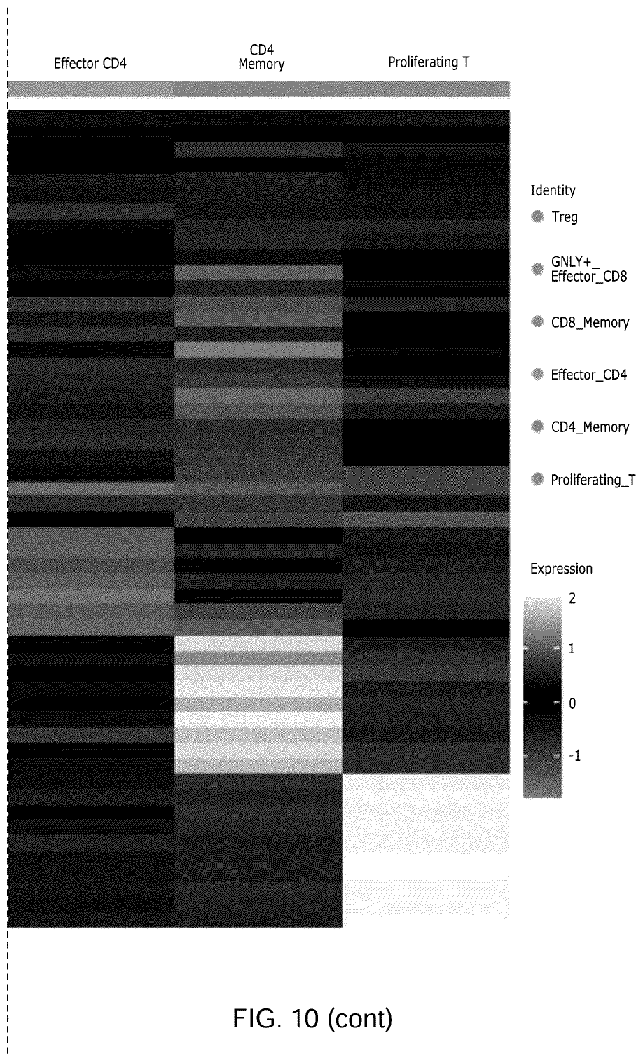

For a more comprehensive analysis specific to T cells, 7,601 T cells were isolated from total cells and reintegrated using reciprocal PCA and CCA for downstream analysis (FIG. 1D). Healthy control 3, along with severe patients 3 and 4, were excluded from analysis due to the extreme reduction of T cell number as suggested in the original study.²² After reintegration, UMAP and Louvain-based optimization were conducted for unsupervised clustering (FIG. 1D). Expression of canonical markers was used for cluster annotation. Proliferating T cells were defined by expression of CD3 and MKI67, Treg by FOXP3; cytotoxic T lymphocyte (CTL) (GNLY+Effector CD8) by CD8A and GNLY; CD8 memory by CD8A and HOPX; effector CD4 by CD4, GZMA, CREM, TMEM173; memory CD4 by CD4, CCR7, IL7R, and HNRNPLL (activation markers); innate T by CD3, TMIGD2 (FIG. 10). CD8 memory cells were significantly diminished in severe patients compared to both healthy controls and moderate patients (FIGS. 1E-F). Similarly, the numbers of innate T cells were drastically decreased specifically in severe COVID-19 (FIG. 1F). Tregs demonstrated a marked reduction in COVID-19 patients (both moderate and severe) compared to healthy. Although not statistically significant, frequencies of effector CD4, and proliferating T cells showed an increasing trend correlated to COVID-19 severity (FIGS. 1E-F). The proportion of CTLs were largely increased in both moderate and severe compared to healthy patients; however, amongst the COVID-19 positive patients, there was a notable decrease in CTLs in the severe vs moderate groups (FIGS. 1E-F). Monocyte abundance was significantly increased in moderate and severe patients whereas macrophage and monocyte derived dendritic cells (mDCs) were decreased. This finding suggests that the differentiation of monocyte lineages may be impaired (FIG. 1B). Mast cells also exhibited a decreasing trend in moderate and severe conditions compared to healthy controls (FIG. 1B).

High-Dimensional Flow Cytometry Identifies Highly Resolved Cell Populations in PBMCs of Patients with COVID-19.

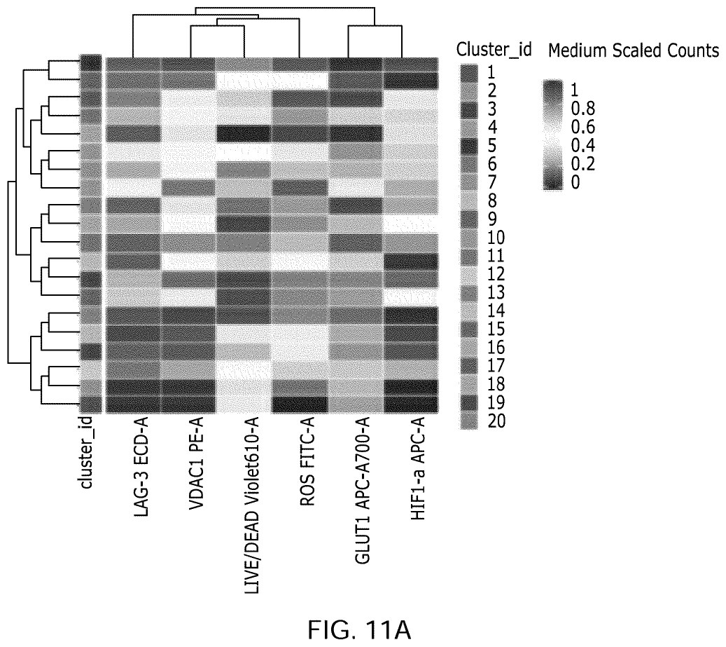

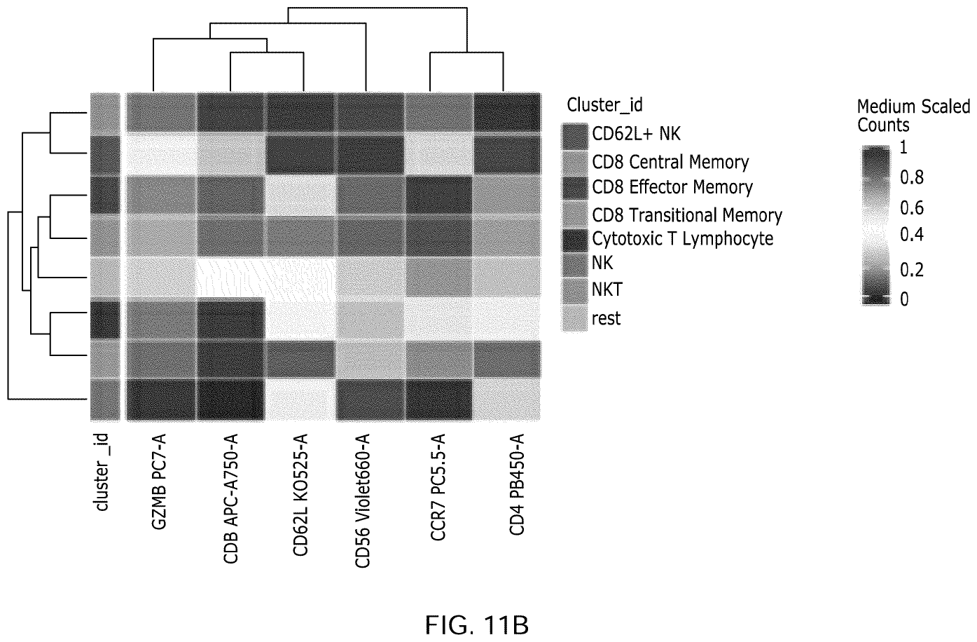

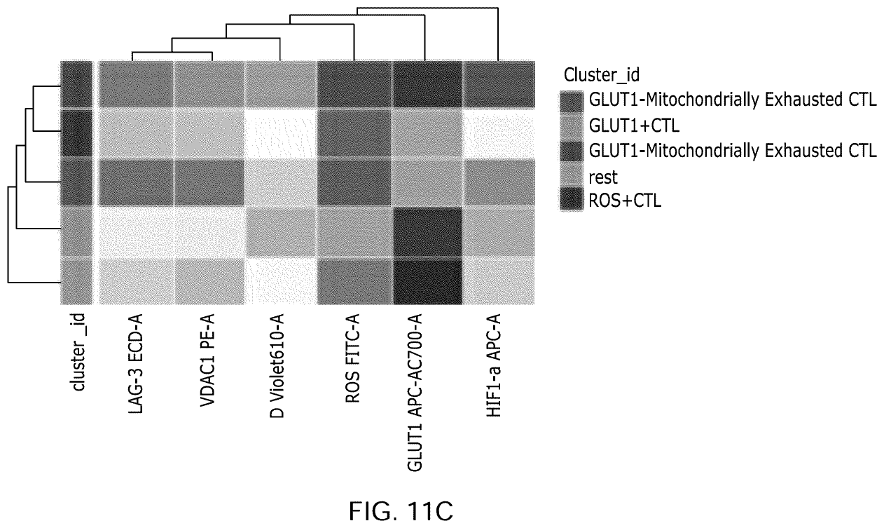

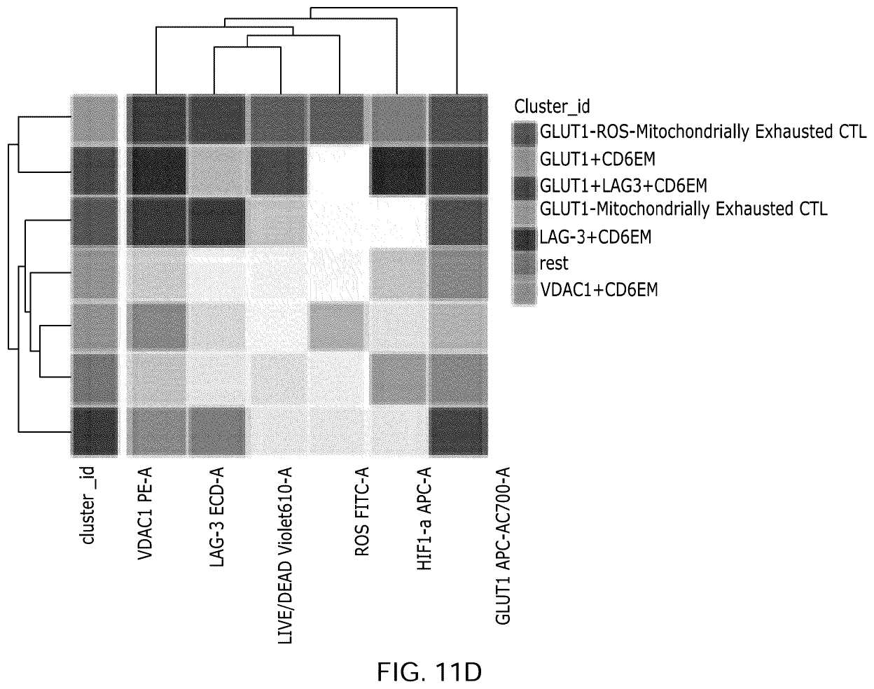

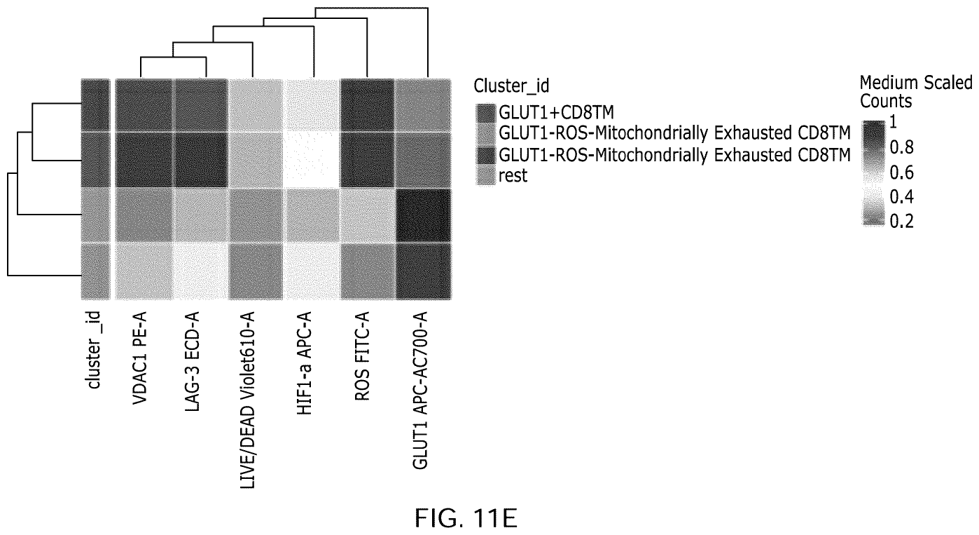

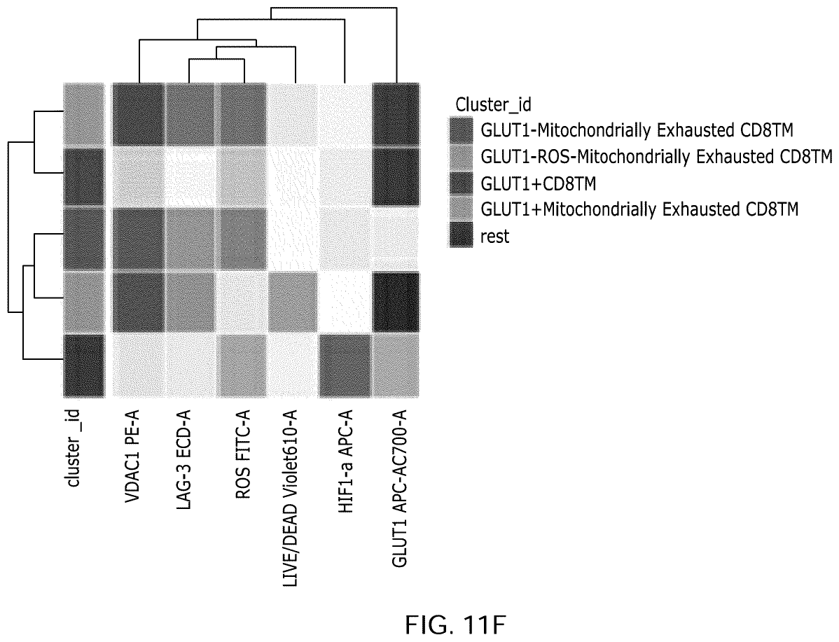

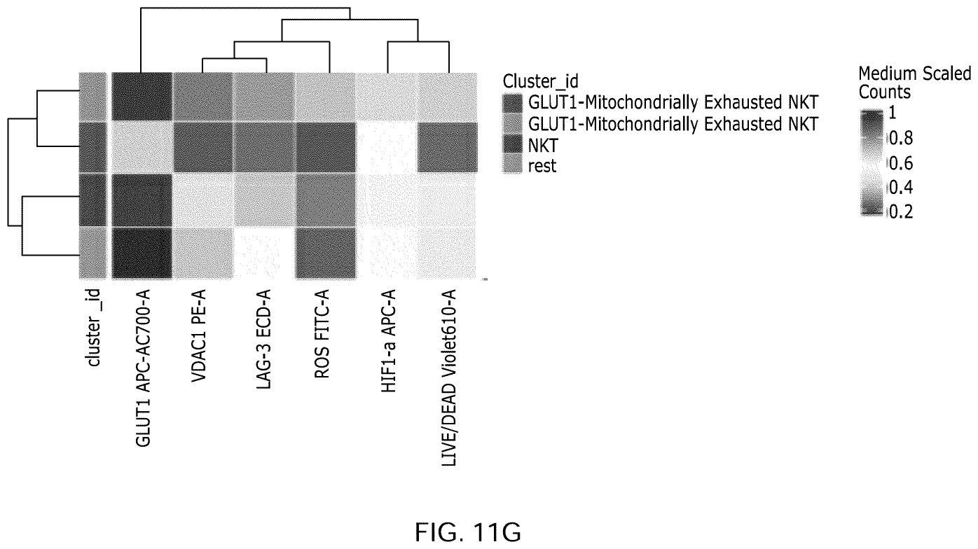

To validate these transcriptome-level observations, PBMCs from hospitalized COVID-19 patients were isolated and high-dimensional immunophenotyping using multiparametric flow cytometry for unbiased identification of low frequency populations was conducted. Freshly isolated PBMCs from 20 COVID-19 and 8 healthy patients were analyzed without stimulation or cryogenic preservation to identify metabolic perturbations in immune cells due to viral infection. Because NK and CD8⁺T cells are critical for effective immunity against SARS-CoV-2, multiparametric flow cytometry to profile the phenotype of these subsets in the PBMCs^{23,24} was first used. Briefly, after quality control and compensation, batch effect was corrected in makers with bimodally distributed expression by the CytoNorm package in R.²⁵ PCA revealed distinct clustering between COVID-19 and healthy samples, suggesting that PBMCs in COVID-19 patients exhibited an abnormal immunophenotype (FIG. 1G). UMAP and FlowSOM²⁶ were used to identify unsupervised clusters (FIG. 1H and FIG. 11A) which were assigned to specific populations based on canonical marker expression (FIG. 11 and FIG. 11B). Cytotoxic T lymphocytes (CTLs) were identified by co-expression of CD8 and GZMB; CD8 central memory by CD8, CD62L, and CCR7; CD8 transitional memory by CD8, CD62L; CD8 effector memory by CD8, CCR7, GZMB; NK by CD56; NKT by CD56, CD8; CD62L⁺NK by CD56, CD62L (Table 2).

TABLE 2

| | Surface Markers Used |
|---|---|
| PBMC Cell Population | for Identification |
| Cytotoxic T Lymphocytes | CD8, GZMB |
| CD8 Central Memory Cells | CD8, CD62L, and CCR7 |
| CD8 Transitional Memory Cells | CD8, CD62L |
| CD8 Effector Memory Cells | CD8, CCR7, GZMB |
| NK Cells | CD56 |
| NKT Cells | CD56, CD8 |
| CD62L⁺ NK Cells | CD56, CD62L |

Nonparametric differential testing showed a significant decrease in the abundance of CTLs, CD8 transitional memory, NKT, and CD62L⁺NK subsets, indicating lymphocytopenia in COVID-19 patients as observed elsewhere^{10,11} (FIGS. 1J-L). Conversely, increases in the percentage of CD8 effector memory and NK cells were found in COVID-19 patients (FIGS. 1J-L). Because the proportion of CD62L⁺NK and CD8 transitional memory cells were inversely correlated to that of NK and CD8 effector memory cells (FIGS. 1J-L), respectively, in COVID-19 patients, it is likely that memory differentiation of NK and CD8⁺T cells is impaired in severe COVID-19. These results suggested that impaired memory formation might be a cause of lymphocytopenia in COVID-19 patients.

### Hypoxia/Anaerobic Glycolysis Axis Induces CD8 T-Cell Lymphocytopenia in COVID-19

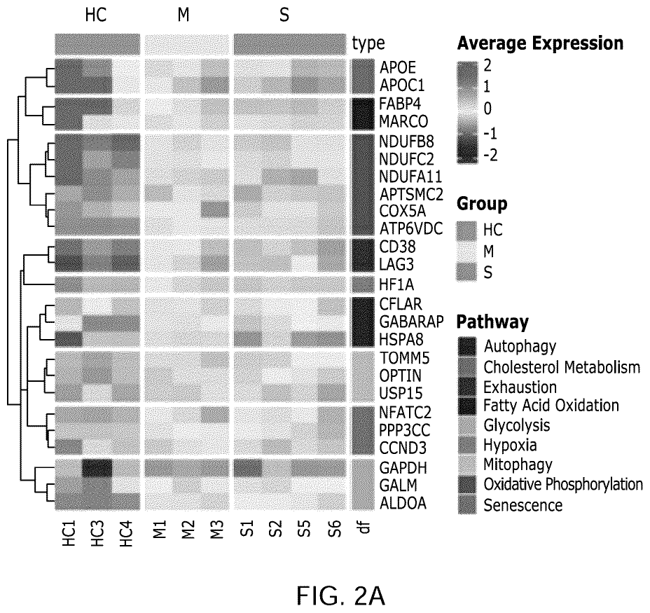

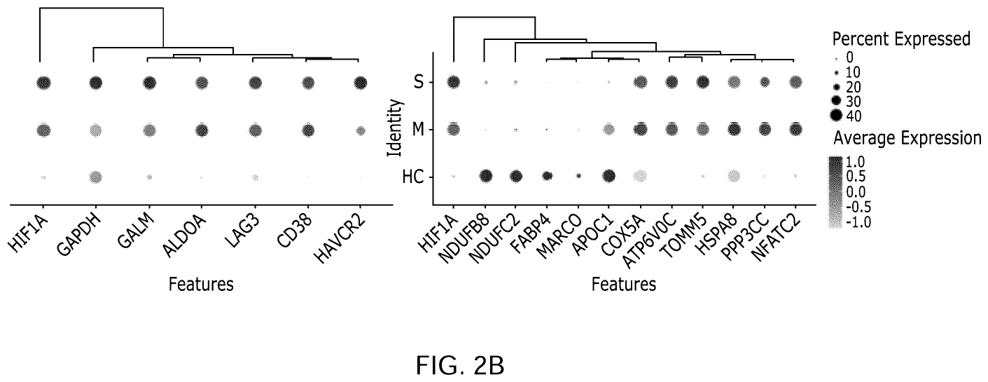

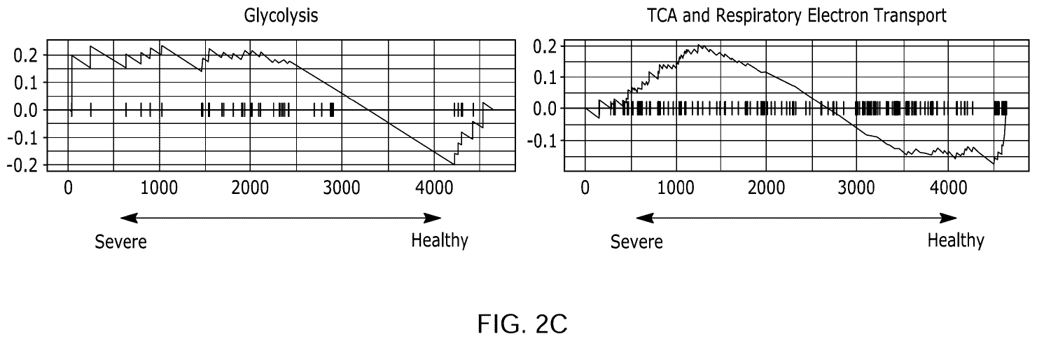

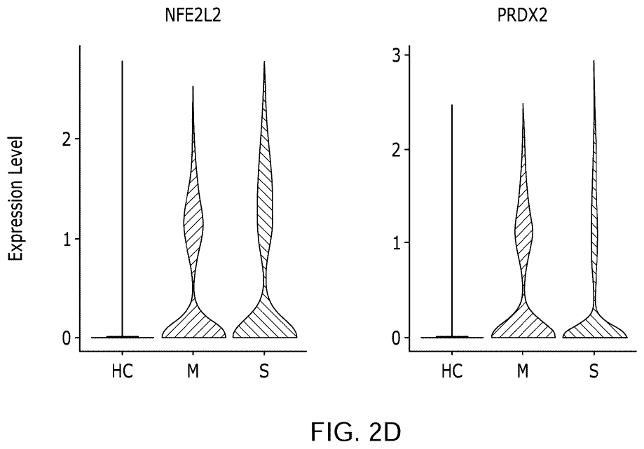

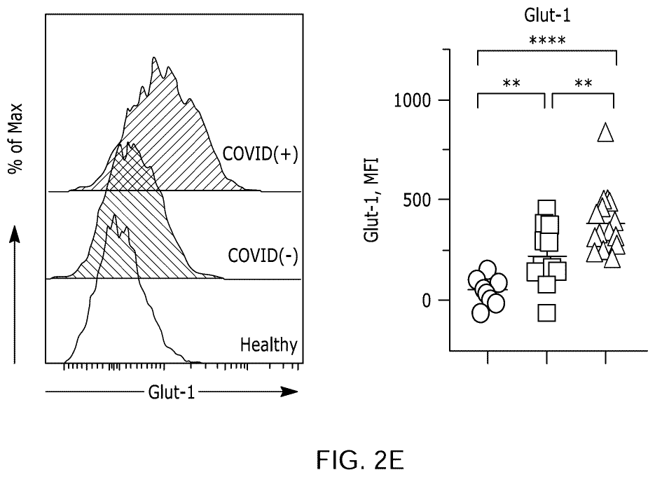

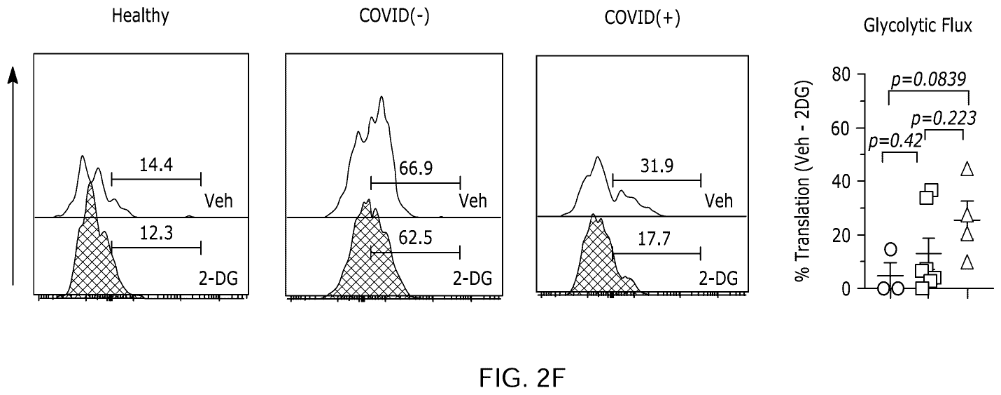

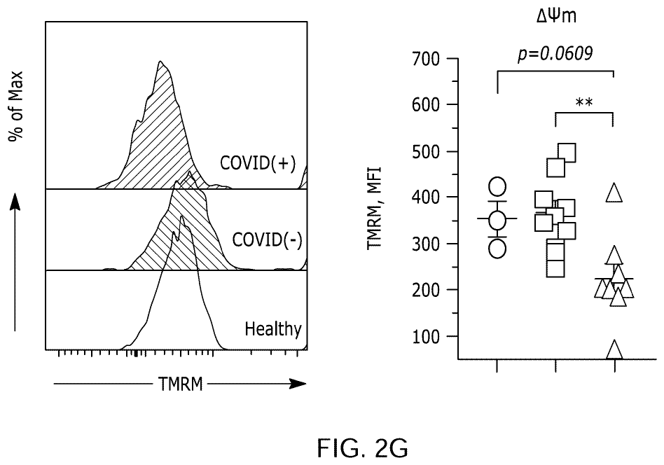

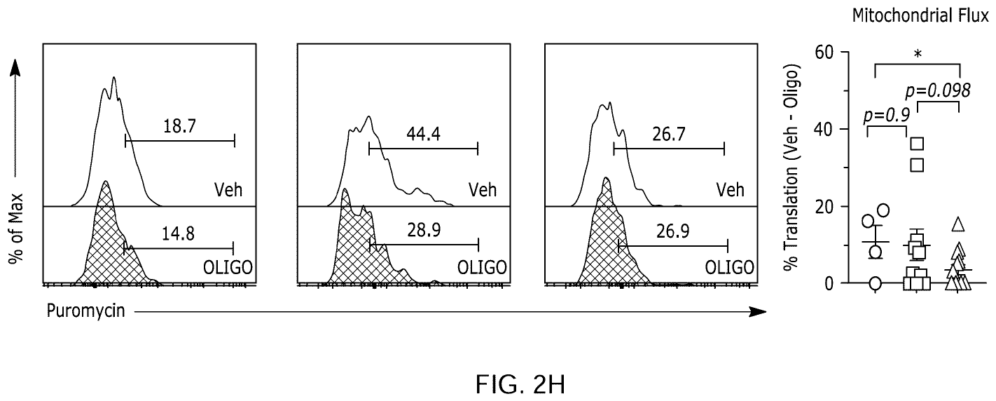

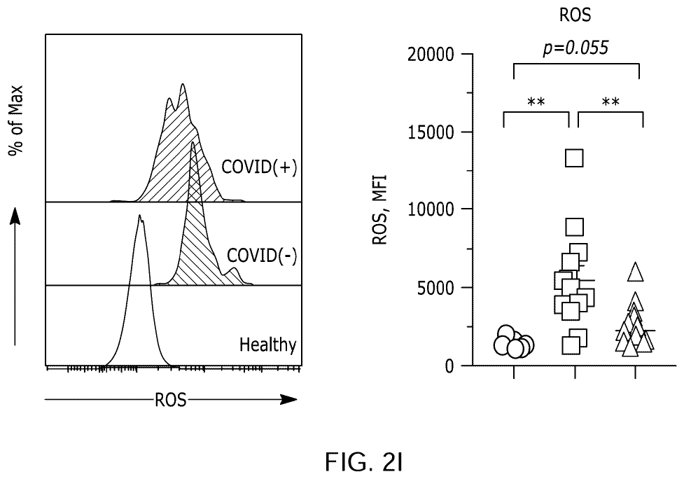

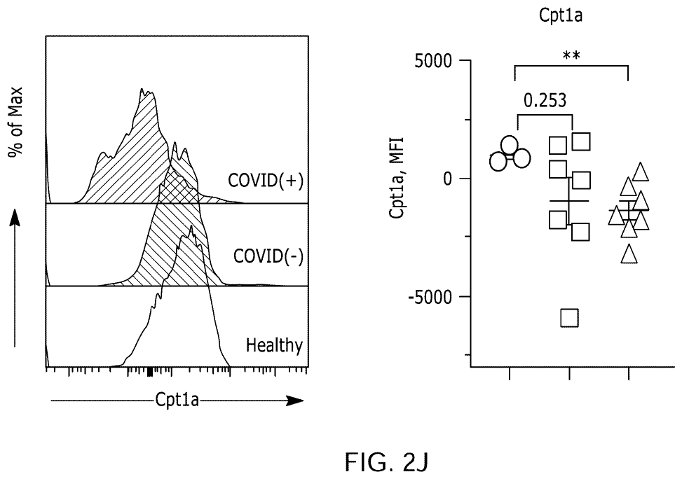

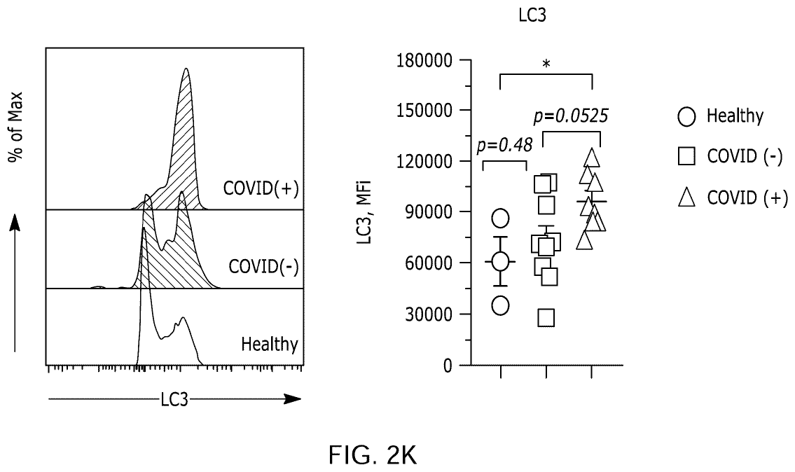

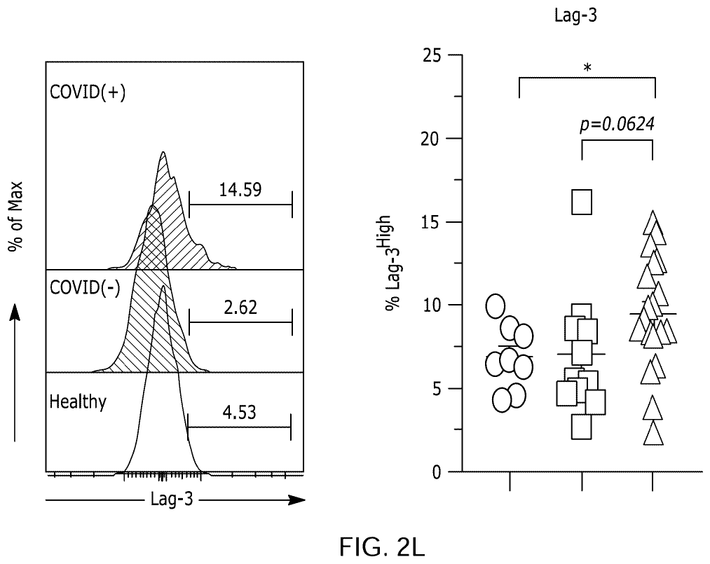

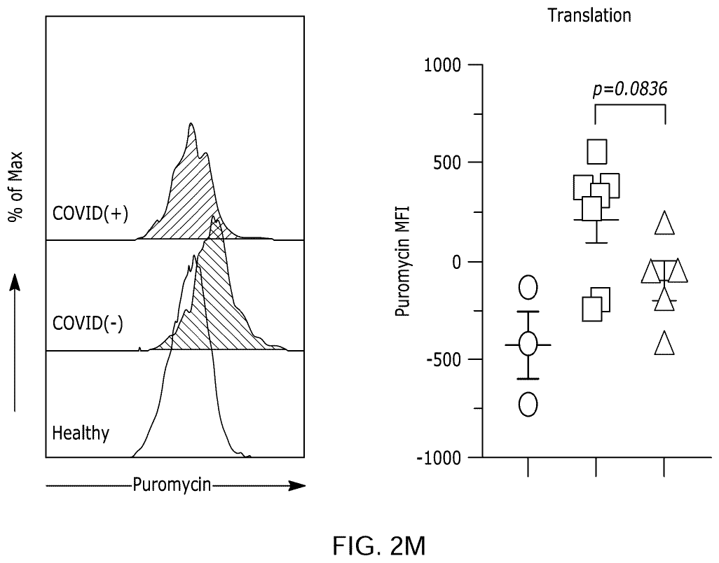

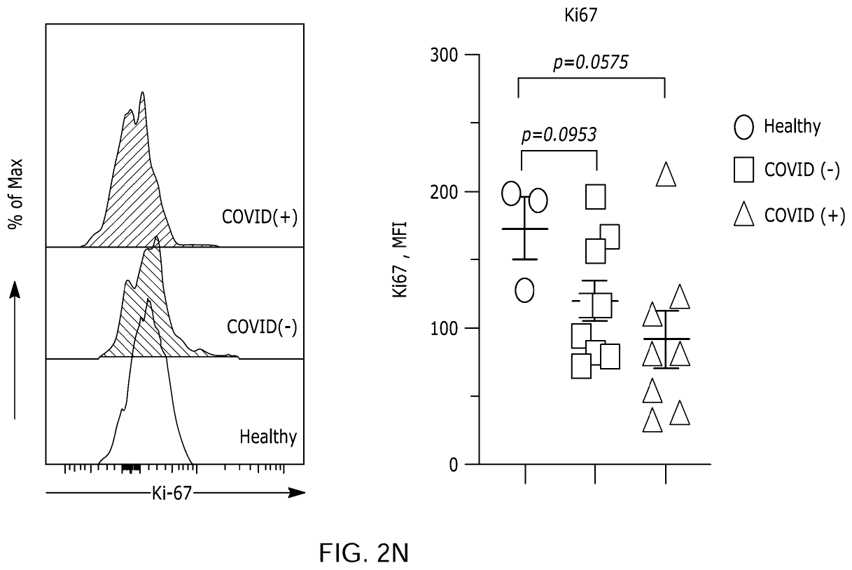

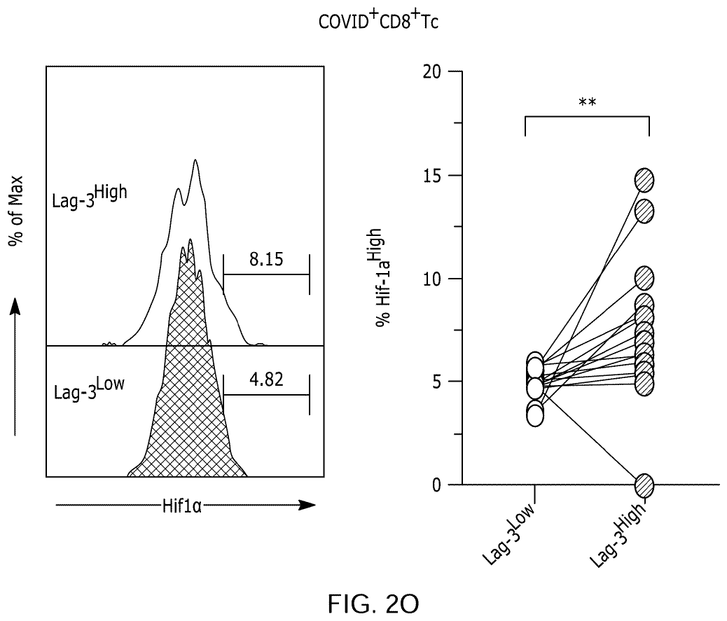

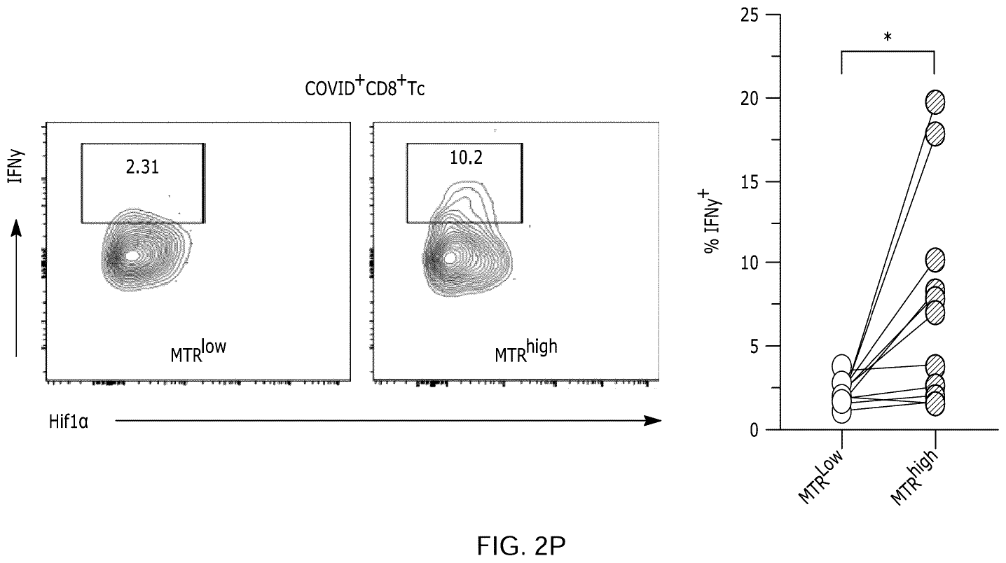

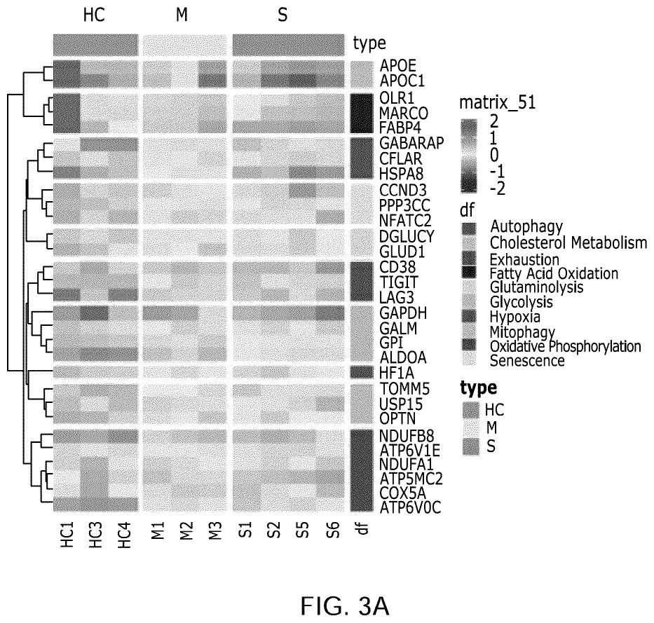

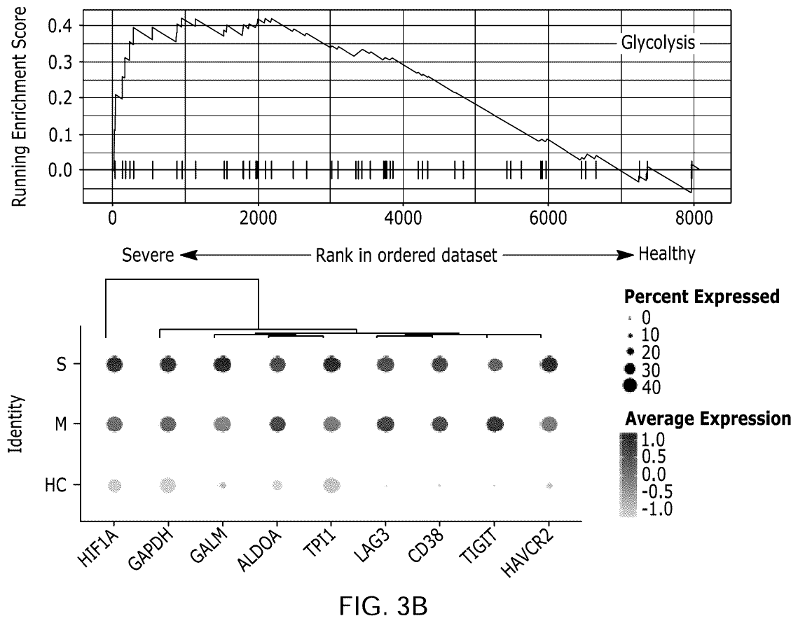

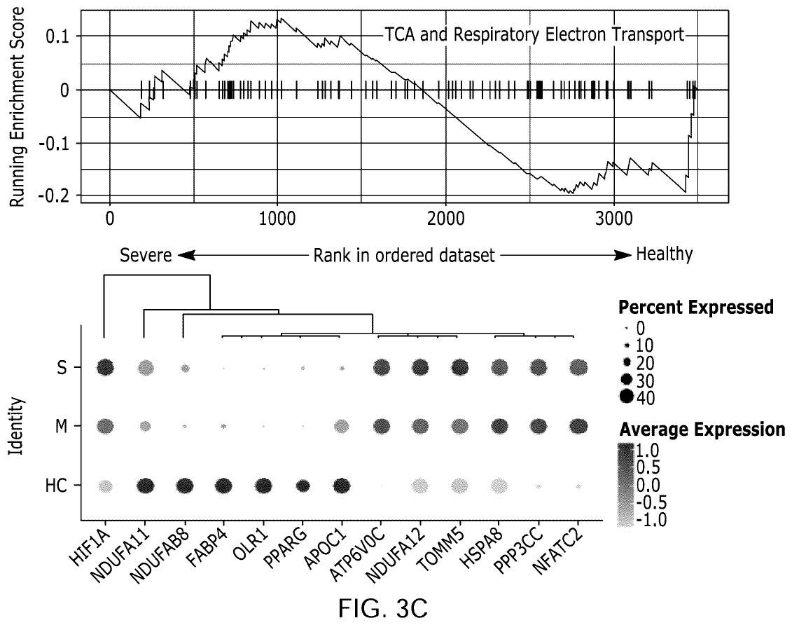

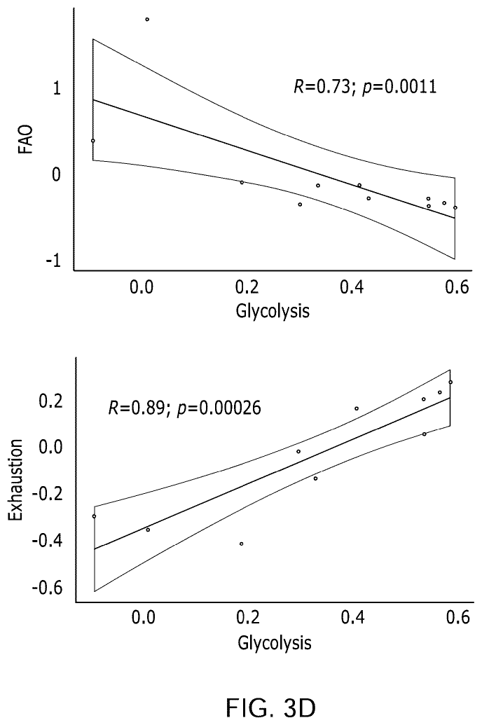

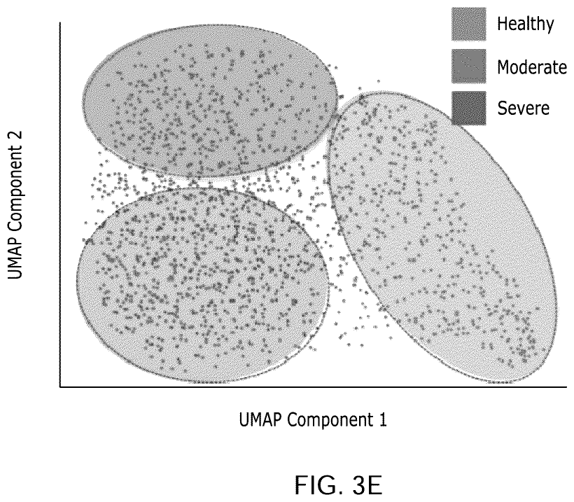

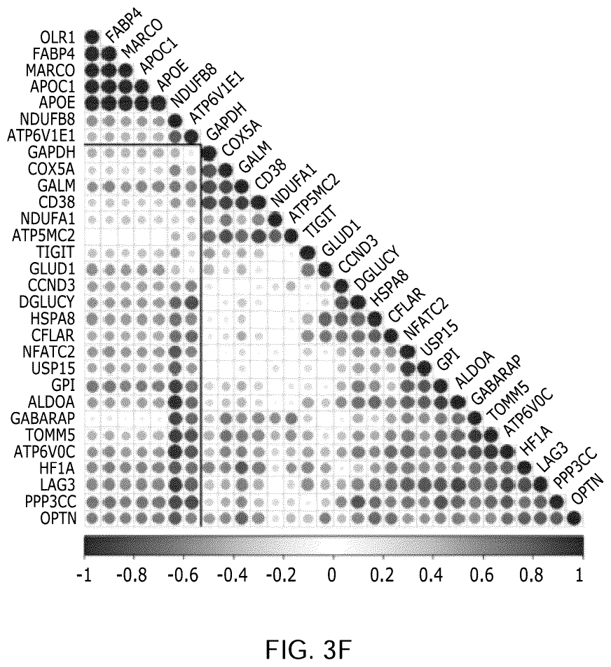

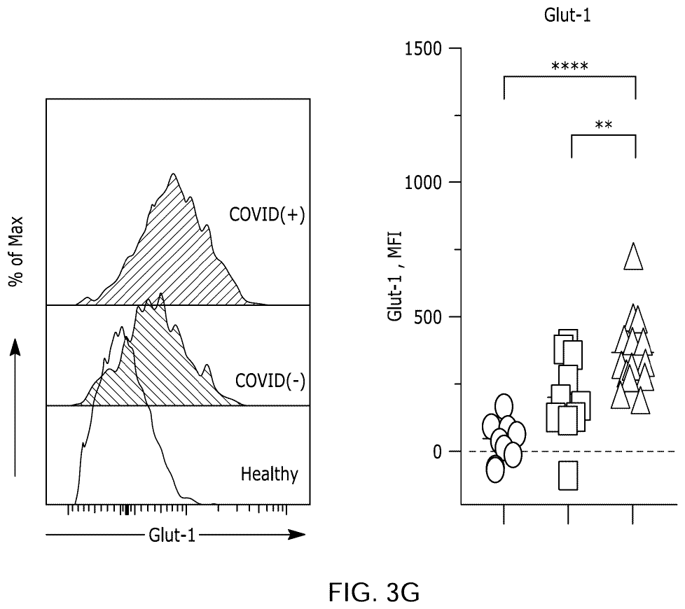

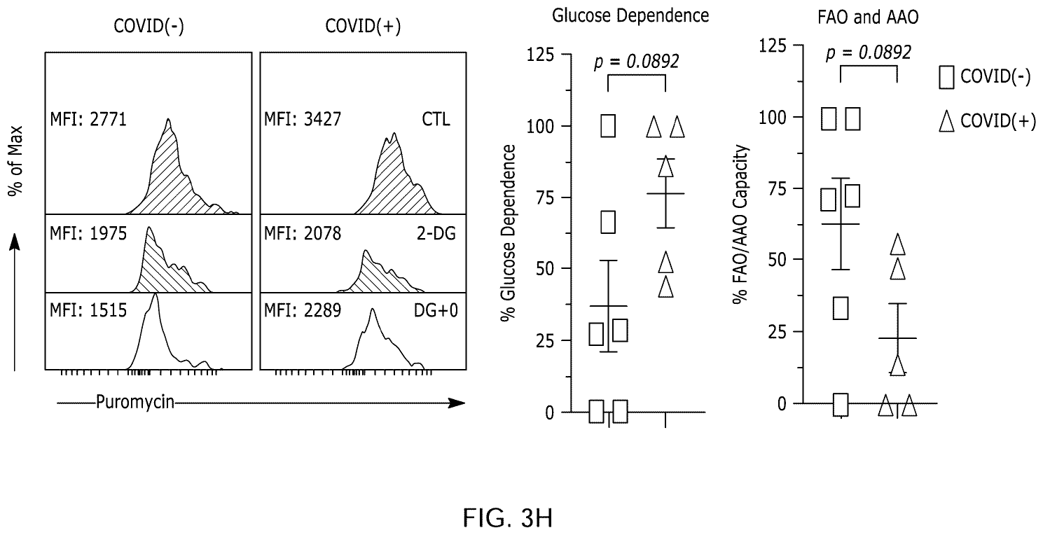

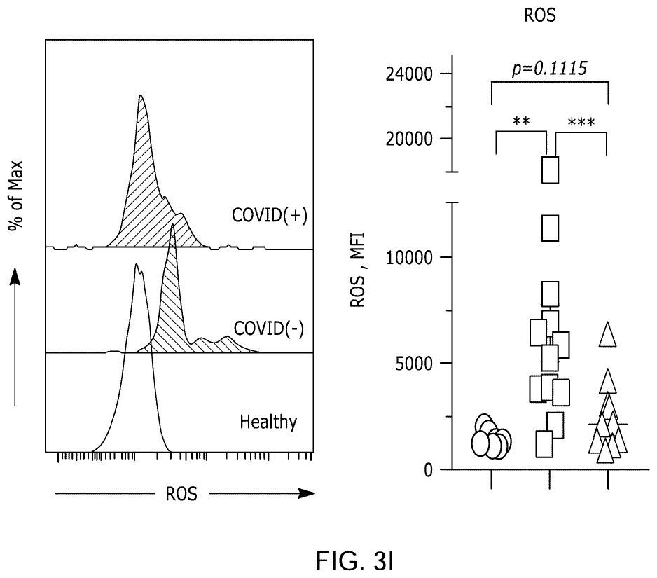

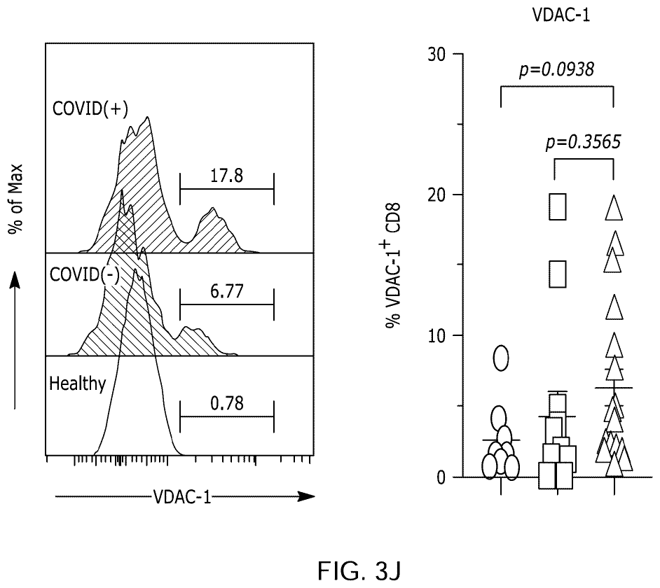

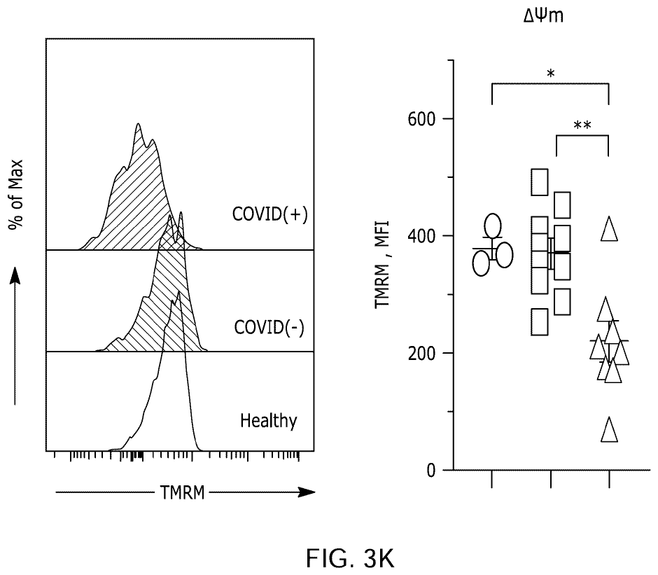

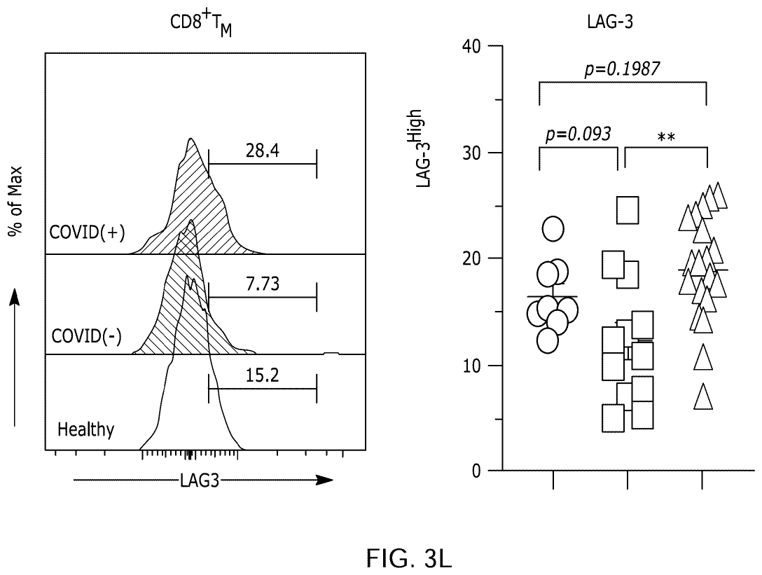

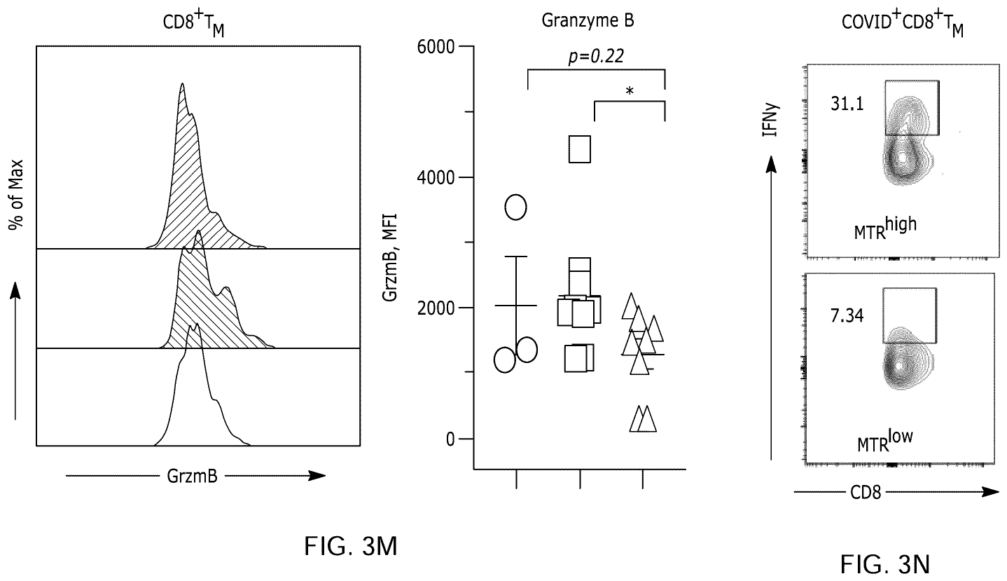

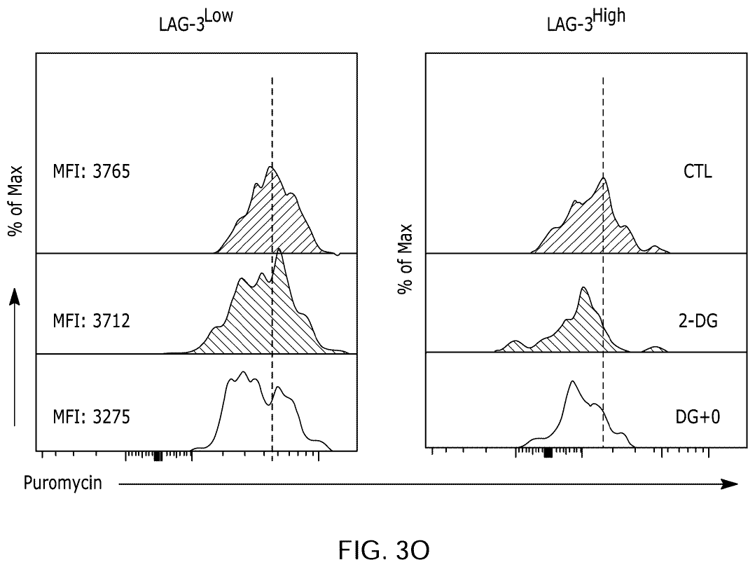

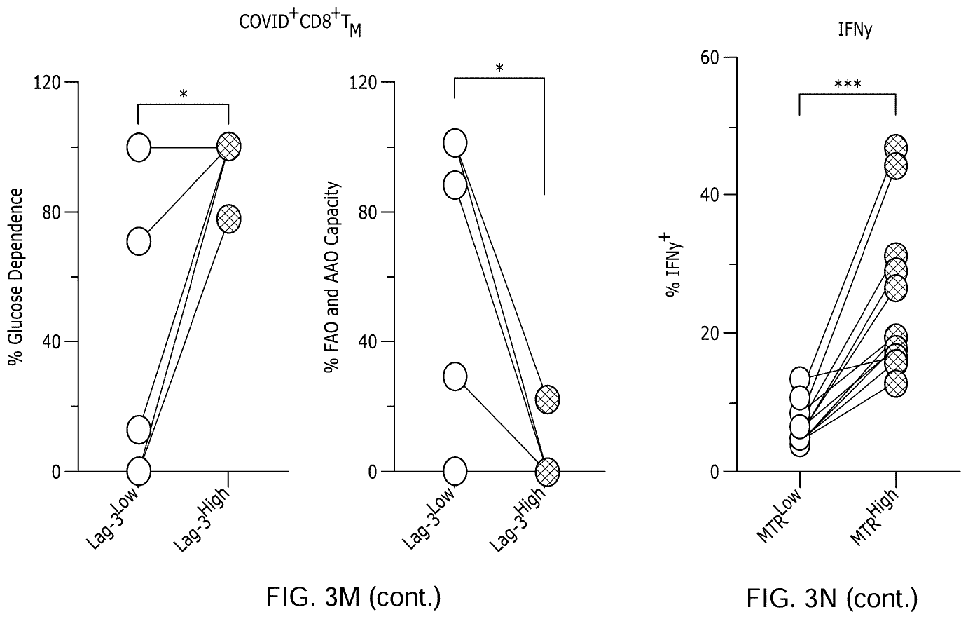

CD8 T-cells are essential for an effective immune response against viral infection²⁷. Thus far, the mechanisms that mediate CD8 lymphocytopenia in SARS-CoV-2-infected patients have not been fully elucidated. Given that cellular metabolism plays an important role in dictating the fate of CD8 T cells during viral infection,^{14,18} metabolic reprogramming in CD8⁺T cells due to SARS-CoV-2 infection was profiled. In the BALF dataset, effector CD8 cells were first subdivided into cytotoxic CD8 cells and others based on canonical markers GNLY, NKG7, and KLRD1, respectively (FIG. 1D). Differential expression analysis and functional gene set enrichment analysis (GSEA) between samples from severe COVID-19 and healthy controls were conducted to identify dysregulated pathways affecting CTL differentiation (FIGS. 2A-C). It was found that hypoxia, glycolysis, mitophagy, autophagy, fatty acid oxidation (FAO), OXPHOS, cholesterol metabolism, cell exhaustion, and cellular senescence were the most overexpressed metabolic pathways in CTLs from COVID-19 patients (FIG. 2A). Hif-1α was clearly overexpressed in CTLs from severe patients, a finding that is concordant with hypoxic and oxygen-deprived conditions present in the BALF microenvironment during severe COVID-19 (FIGS. 2A-C). GSEA analysis demonstrated a higher basal glycolytic state for CTLs in severe COVID-19 patients (FIG. 2C). Increased glycolytic activity was accompanied by overexpression of key glycolytic regulators genes (GAPDH, GALM, ALDOA) (FIGS. 2A-C). During normal CTL differentiation, cells preferentially shift from OXPHOS towards mTOR/ATK-mediated aerobic respiration to sustain increased bioenergetic demand and augmented mitochondrial biosynthesis.²⁸ Unexpectedly, a significant decrease in the expression of genes encoding NADH oxidoreductases (NDUFB8, NDUFC2, NDUFA11) was observed, and also an increase in the expression of genes encoding cyclooxygenases and transcripts responsible for ATP synthesis was observed (FIGS. 2A-C). Furthermore, increased expression of genes regulating oxidative stress (NFE2L2, PRDX2) (FIG. 2D), accompanied with an upregulation of senescence and mitophagy (FIG. 2A) were observed. An accompanying significant decrease in FAO and lipid metabolism in CTLs from severe patients was observed in 4 associated genes (FABP4, APOC1, APOE, MARCO) (FIGS. 2A-B). Together, these results suggest that hypoxic conditions linked to Covid-19-pulmonary dysfunction triggers impaired FA metabolism and oxidative stress, which induces mitochondrial dysfunction. Given that a decrease in NADH oxidation and increased NAD⁺ levels is associated with impaired cytokine secretion, proliferation, survival and oxidative stress,^{29,30} NAD⁺ depletion might mediate CD8 T cell exhaustion in patients with severe COVID-19.¹¹ Interestingly, the expression of CD38, an NAD⁺ hydrolase associated with T cell exhaustion,³⁰ was significantly increased (FIG. 2A). It was hypothesized that hypoxia induced CD38 expression, coupled with a microenvironment consistent with prolonged anaerobic glycolysis indicated by hyperlactatemia,¹⁰ local nutrient deprivation, and excessive cytokine secretion, drives cellular exhaustion and lymphocytopenia in CTLs in COVID-19. CTLs from severe COVID-19 patients expressed significantly higher levels of LAG3, a canonical T cell exhaustion marker, as compared to healthy controls (FIGS. 3A and D). Hierarchical clustering indicated that ALDOA and exhaustion markers clustered closely together (FIG. 2B) thus bolstering the putative causal relationship between hypoxia/anaerobic glycolysis and cellular exhaustion.

Next, building upon the transcriptomics results of transcriptomic analysis by evaluating expression of various proteins by PBMCs from COVID-19 ICU patients was attempted. Cytotoxic CD8 T cells were identified from CD8 T cells in PBMCs by co-expression of CD8 and GZMB (FIG. 1B and FIG. 10). Again, as demonstrated in FIG. 1C the percentage of CTLs was significantly decreased in COVID-19 patients as compared to healthy individuals. The glucose transporter 1 (GLUT1) governs glycolytic flux in lymphocytes.³¹ Striking increases in median fluorescent intensity (MFI) of CTL GLUT1 expression and frequency of GLUT1⁺ CTL in COVID-19 patients was observed (FIG. 2E). These results suggest that COVID-19 CTLs display increased dependence on glycolysis to meet bioenergetic demands. Increased oxidative stress was also observed in COVID-19 PBMC CTLs, as indicated by higher frequency of reactive oxidative species (ROS)^{high} CTLs in PBMCs of COVID-19 patients (FIG. 2F). The median expression of ROS and voltage-dependent anion-selective channel 1 (VDAC-1) was also significantly increased in GLUT1⁺ CTLs (FIG. 2G). Given that VDAC1 is associated with mitochondrial cell death signaling,³² these results indicate that upregulated glycolysis in CTLs is associated with oxidative stress and mitochondrial dysfunction in COVID-19 PBMC CTLs. Furthermore, the large increase in LAG-3 expression revealed the exhausted phenotype of COVID-19 PBMC CTLs (FIG. 2G). To validate this proposed relationship, unsupervised clustering with FlowSOM on CTLs using the expression of GLUT1, ROS, LAG3, and VDAC1 to glean insight into the functional metabolic state of CTLs (FIG. 2H and FIG. 11C) was performed. A unique cluster with high expression of all markers GLUT1, LAG3, ROS, and VDAC1 was identified, which was designated as GLUT1⁺ mitochondrially exhausted CTLs (FIG. 2H and FIG. 11C). This cell population was significantly enriched in PBMCs of patients with severe COVID-19 (FIG. 2I). It was inferred that CTLs from COVID-19 patients are severely exhausted and exhibit mitochondrial dysfunction and metabolic dysregulation. These results confirmed that excessive aerobic glycolysis, accompanied by mitochondrial damage, causes CTL exhaustion in the blood of severe COVID-19 patients. CTLs in severe COVID-19 appear to exhibit a metabolic shift to greater reliance on anaerobic glycolysis and oxidative stress, resulting in impaired mitochondrial function and cellular exhaustion. These effects can lead to lymphocytopenia and attenuated viral clearance. Importantly, while CTLs from both severe and healthy donors are metabolically reprogrammed to exhibit increased glycolysis, the underlying cellular signaling and mechanistic factors are different. Unlike CTLs from healthy donors, which acquire an aerobic glycolysis phenotype via increasing mTOR/AKT signaling after TCR activation,³³ CTLs from severe COVID-19 patients underwent a hypoxia-mediated metabolic shift toward anaerobic glycolysis. While aerobic glycolysis shift seen in healthy CTLs to augment effector cytokine production and cytotoxic activity; this metabolic reprogramming triggers cellular exhaustion, mitochondrial dysfunction, and consequently impairs CTL-mediated anti-SARS-COV-2 activity.

### COVID-19-Induced Metabolic Dysfunction Drives Memory T Cell Exhaustion

Memory T cells are antigen-specific T cells that persist after the initial viral infection,³⁴ which can quickly differentiate into effector subtypes upon reinfection.³⁵ Elucidating how metabolism influences memory formation during the viral infection will also be useful for understanding how metabolic disorders might alter the immune response to Covid-19 vaccination. The metabolic phenotype of CD8 memory cells during SARS-CoV-2 infection was profiled and analyzed.

Effector CD8 cells in BALF were identified as memory cells based upon the expression of canonical markers including HOPX and SELL (FIG. 11A). In addition to the metabolic pathways that were dysregulated in COVID-19 CTLs, glutaminolysis-related genes were found to be differentially expressed in CD8 memory cells (FIG. 3A). Generally, during CD8 memory cell differentiation from CTLs, metabolic reprogramming leads to increases in FAO, OXPHOS and mitochondrial metabolic fitness, while decreasing glycolytic capacity.³⁶ However, significantly increased glycolytic potential in CD8 memory cells from severe COVID-19 patients was observed as evident by upregulation of critical glycolytic genes (GALM, GAPDH, GPI, ALDOA) (FIGS. 3A-B). A moderate decrease in OXPHOS was also revealed by the downregulation of genes responsible for NADH oxidation (FIGS. 3A-B). Meanwhile, genes coding for regulators of lipid uptake and FAO (OLR, MARCO, FABP4, APOE, APOC1) were significantly downregulated in CD8 memory cells of severe COVID-19 patients (FIG. 3A). Hif-1α expression was significantly upregulated indicating hypoxic conditions in the BALF microenvironment may drive CD8 memory cells toward anaerobic glycolysis (FIG. 3A). Pearson correlation analysis revealed a negative correlation (R=−0.73, p=0.011) between module scores for glycolysis and FAO (FIG. 3C) thus suggesting that prolonged hypoxia-induced anaerobic glycolysis is responsible for decreased mitochondrial fitness. Consistently, genes of cellular senescence and mitophagy were significantly increased (FIG. 3A), suggesting that CD8 memory cells augmented these pathways to produce necessary catabolic intermediates and sustain bioenergetics demands. Interestingly, increased expression of the GLUD1 and DGLUCY genes mediating glutamate oxidation were also identified, thus indicating that that CD8 memory cells increase glutaminolysis to maintain TCA cycle function in response to a reduced lipid uptake and FAO (FIG. 3A).

Next, a Pearson correlation analysis on averaged pseudo-bulk data was conducted using a panel of 30 identified key metabolic genes (Table 3) differentially expressed in severe COVID-19 patients.

TABLE 3

| Cell type | Panel for metabolic phenotype-based clustering |
|---|---|
| CTL | APOE, APOC1, FABP4, MARCO, NFUFB8, NDUFC2, NDUFA11, ATP5MC2, |
| | COX5A, ATP6V0C, CD38, LAG3, HIF1A, CFLAR, GABARAP, HSPA8, |
| | TOMM5, OPTN, USP15, NFATC2, PPP3CC, CCND3, GAPDH, GALM, ALDOA |
| CD8 | APOE, APOC, OLR1, MARCO, FABP4, GABARAP, CFLAR, HSPA8, CCND3, |
| Memory | PPP3CC, NFATC2, DGLUCY, GLUD1, CD38, TIGIT, LAG3, GAPDH, GALM, |
| | GPI, ALDOA, HIF1A, TOMM5, USP15, OPTN, NDUFB8, OSP15, OPTN, NDUFB8, |
| | ATP6V1E1, NDUFA1, ATP5MC2, COX5A, ATP6V0C |
| Epithelial | IGFBP3, CDKN1A, GADD45B, ADH1C, ALDOA, GAPDH, PCK2, ENO1, |
| | ALDH1A3, GABARAP, SQSTM1, HIF1A, CTSB, CTSL, NDUFC2, NDUFB8, |
| | ATp5PO, UBB, MDH1, SDHC, ACADM, ACI2, SLC27A2, FABP6, APOC1, |
| | VDAC3 |
| NKT | FABP4, OLR1, SCP2, ACADM, HIF1A, GAPDH, LDHA, TPI1, PGAM1, ALDOA, |
| | LAG3, CD44, CD38, NDUFA11, NDUFA13, NDUFB8, ATp5MC2, NDUFA12, |
| | ATP6V0C, COX5A, CTSL, CFLAR, ITPR1, UBB, KRAS, CSNK2A1, SLC25A5, |
| | GAFF45B, JUN, PRF1, IFNG, TNFSF10, HCST, LRC2, KLRC1, APOE, APOC1 |

A significant positive correlation was observed between expression of genes of glycolysis, mitophagy, senescence, and glutaminolysis, which was negatively correlated with genes regulating FA and NADH oxidation (FIGS. 3C-D). Furthermore, increased expression of CD38 suggested that decreased NAD and increased Hif-1α-triggered anaerobic glycolysis are also responsible for CD8 memory lymphocytopenia. As expected, canonical exhaustion markers LAG3 and TIGIT were upregulated in CD8 memory cells from severe COVID-19 patients (FIG. 3A). Pearson correlation analysis of module scores confirmed a strong positive correlation for glycolysis and exhaustion (R=0.85, p=0.00026) (FIG. 3C).

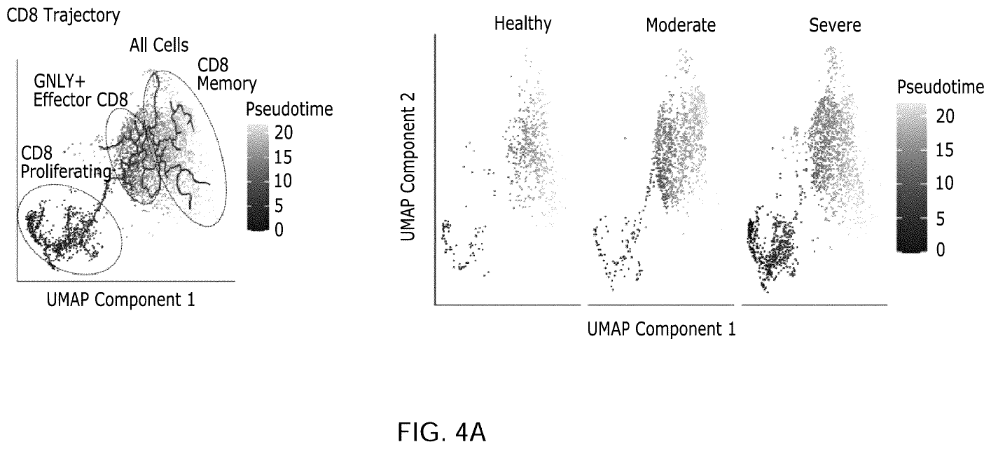

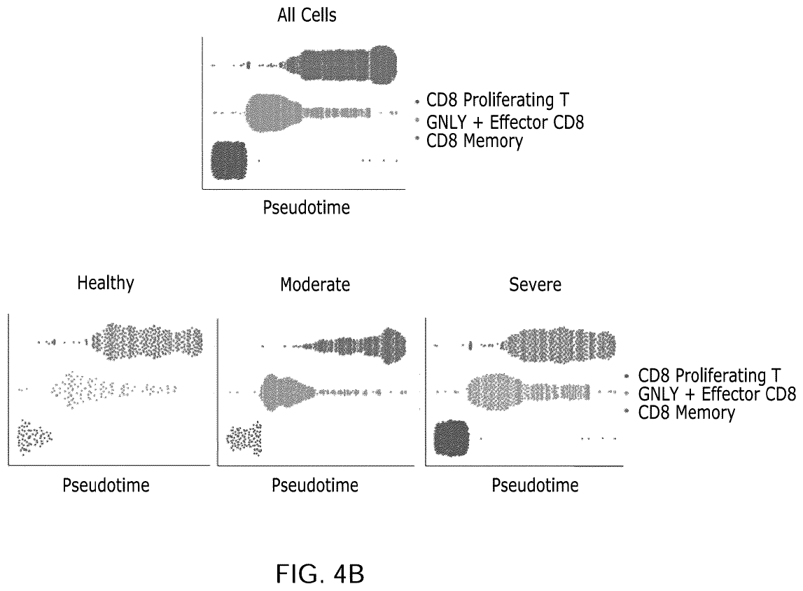

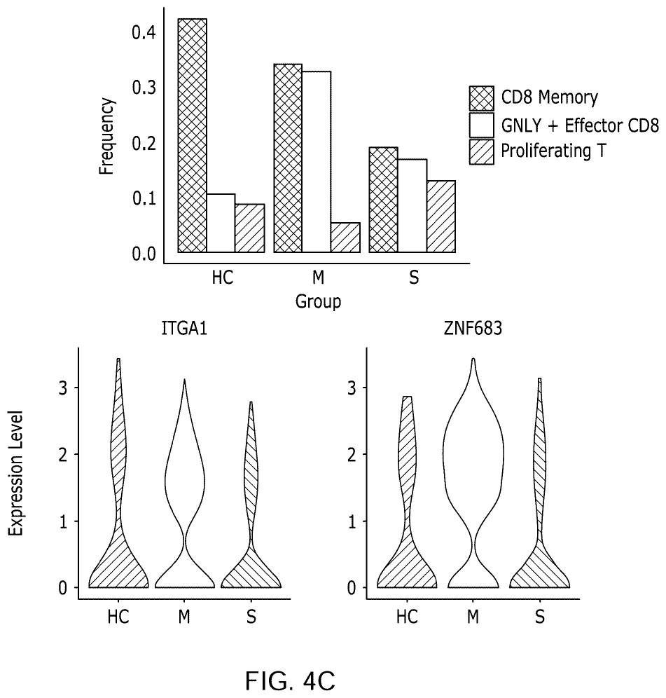

Because SARS-CoV-2-specific CD8 T cells are inversely correlate with COVID-19 severity,³⁷ the possibility that the metabolic phenotype of CD8 memory cells could be used as a predictive indicator of disease severity was explored. UMAP was performed again on the CD8 memory cell population using only the panel of 30 key identified metabolic genes (FIG. 3E and Table 3) to see if unsupervised clustering at a single-cell resolution could differentiate cells based upon the severity of the COVID-19 patients from which the cells were derived. Samples demonstrated distinct clustering and separation by disease severity (FIG. 4C-D), indicating that memory CD8 metabolism alone could indeed predict the disease severity.

CD8 memory cells are often categorized into three major subsets: CD8CM, CD8TM, and CD8EM based upon canonical marker expression.³⁸ Briefly, CD8CM were defined by CD8, CD62L, CCR7; CD8TM by CD8, CCR7, GZMB; CD8EM cells by CD8, CD62L, and GZMB. A significant increase in population of GLUT1^{high} CD8EM cells in PBMCs from COVID-19 patients was observed which corroborates the earlier conclusion that CD8EM cells in COVID-19 exhibit robust glycolytic activity (FIG. 3F). GLUT1⁺ CD8EM cells had increased expression of Hif-1α and VDAC1 (FIG. 3G) suggesting that the oxygen-deprivation associated with COVID-19 infection also modulates mitochondrial dysfunction and drives anaerobic glycolytic reprogramming in CD8EM cells. Interestingly, there was no difference in the expression of LAG-3 in the percentage of identified GLUT1⁺ mitochondrially exhausted CD8EM by unsupervised clustering (FIGS. 3G-H). Therefore, at this point in their differentiation trajectory, while CD8EM cells do not exhibit an exhausted phenotype, a clear metabolic shift to anaerobic glycolysis accompanied by dysregulated mitochondrial function is observed (FIGS. 3F-G). Furthermore, a significant increase in ROS^{high}CD8TM cells was seen (FIG. 31). Unsupervised clustering showed an abundance of GLUT1⁺ mitochondrially exhausted CD8TM, which was positive for GLUT-1, ROS, LAG-3, and VDAC1 in COVID-19 (FIG. 3J). A significant increase in percentage of GLUT1⁺ and ROS^{high} CD8CM cells (FIG. 3K) was also observed. The frequency of GLUT1⁺ mitochondrially exhausted CD8CM cells showed clear increasing trend, albeit not statistically significant, in COVID-19 PBMCs (FIG. 3L). Thus, although CD8 memory cell lymphocytopenia is not evident in COVID-19 patient PBMCs, all three detected memory subpopulations exhibit a hypoxia-mediated metabolic reprogramming to anaerobic glycolysis coupled with significant mitochondrial dysfunction. Additionally, this differentially abundant population of exhausted cells with clear mitochondrial dysfunction were detected in both CD8CM and CD8TM populations, although not detected in CD8EM (FIGS. 31 and 3L). Given that the abundance of CD8TM was solely decreased in COVID-19 (FIG. 1K) and CD8TM display an exhausted phenotype, it is likely that metabolically driven exhaustion in CD8 memory cells is tightly linked to lymphocytopenia in COVID-19.

Overall, these results demonstrate that hypoxia and nutrient deprivation within the microenvironment thwart CD8 memory cells of COVID-19 patients from relying on FAO and mitochondrial metabolism, thereby forcing metabolic reprogramming towards anaerobic glycolysis and mitophagy to satisfy their bioenergetics demands. Prolonged glycolysis coupled with increased Hif-1α signaling, leads to a nutrient depleted, high lactate milieu, which promotes increased cellular exhaustion and senescence, impaired CD8 memory cell formation and ultimately lymphocytopenia. Consequently, a decreased CD8-mediated anti-viral response will be mustered upon re-exposure to the SARS-CoV-2 pathogen.

### Metabolic Dysregulation in COVID-19 Dictates Impaired CD8 T-Cell Differentiation

Cellular metabolism is known to be dysregulated and linked to cytokine release syndrome (CRS), which modulates cell differentiation.³⁹ Thus, understanding the impact of immunometabolism on T cell differentiation is crucial for the efficacy of both COVID-19 therapeutics and vaccines. The trajectory of CD8 cell differentiation during SARS-CoV-2 infection in the BALF microenvironment was analyzed (FIG. 4A). CD8 cells were fractionated and monocle 3 was used to define a trajectory on UMAP embeddings for the entire population of CD8 cells. After the trajectory was defined, cells were then ordered in pseudotime²⁰ to evaluate differentiation kinetics (FIG. 4A). Differential analysis revealed decreased pseudotime values for CD8 memory cells from severe compared to moderate and healthy patient (FIGS. 4A-B). This indicated that CD8 memory cells are stuck along the differentiating trajectory and are unable to reach the terminal state during severe COVID-19 infection (FIGS. 4A-B). During viral infection, circulating effector memory cells migrate to the infected tissue and differentiate into tissue-resident memory (Trm) cells to provide the first response defense against reencounter of the pathogen.⁴⁰ Indeed, CD8EM cells from moderate patients were found to express higher tissue residence phenotype (i.e., increased expression of ZNF683 and ITGBN) as compared to CD8EM cells from severe patients (FIG. 4D). This novel finding suggests that impaired effector CD8 differentiation results in decreased Trm formation, which subsequently leads to impaired viral clearance in severe COVID-19. This mechanism, along with impairment of mitochondrial metabolic fitness and excessive anaerobic glycolysis in CD8 cell subsets, can drive CD8 cell dysfunction.

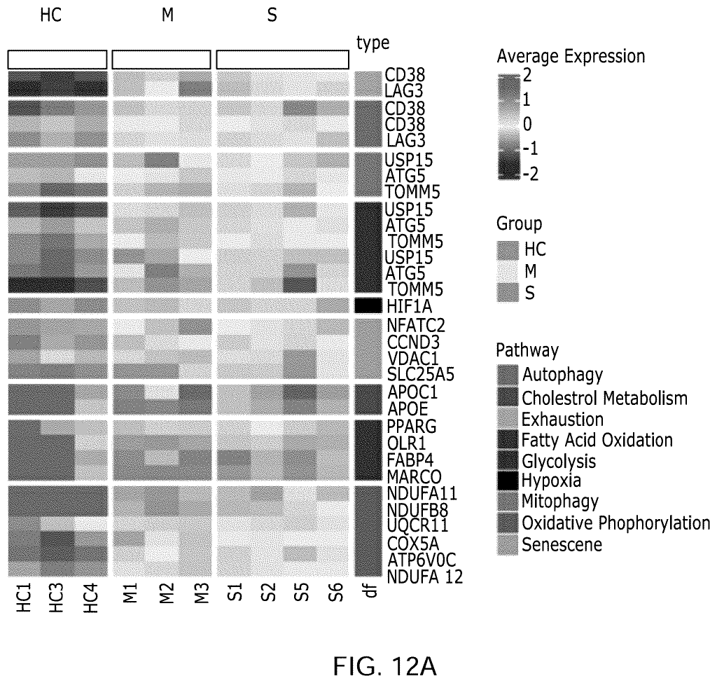

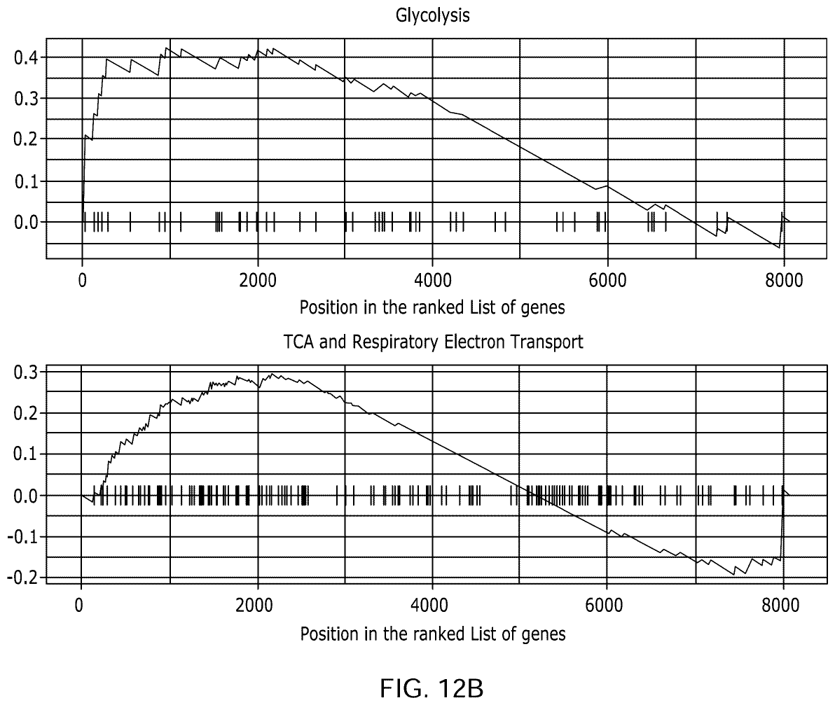

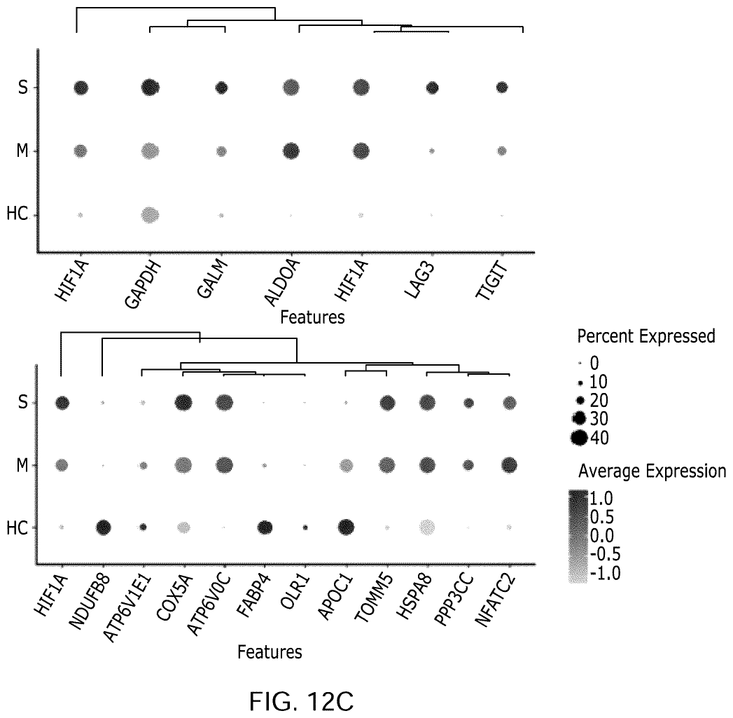

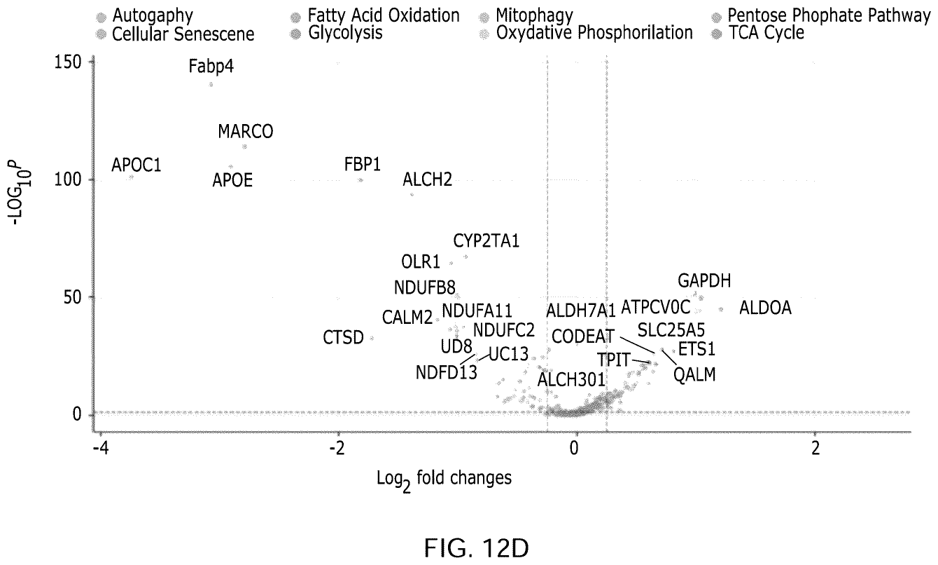

There was also a significant increase in the proportion of proliferating T cells along with a decreased frequency of activated CD8 cells in severe compared to moderate patients (FIG. 4D). Hence, the transition of proliferating T cell (CD8 and MKI67) into activated CD8 cells also appears to be impaired in severe COVID-19. Similar to CTLs and memory CD8 cells, proliferating T cells in severe patients are metabolically reliant on anaerobic glycolysis and mitophagy, while suppressing FAO and OXPHOS (FIGS. 12 A-D). Consequently, proliferating T cells also become functionally exhausted (FIGS. 12C-D) and their differentiation into effector CD8 cells is compromised.

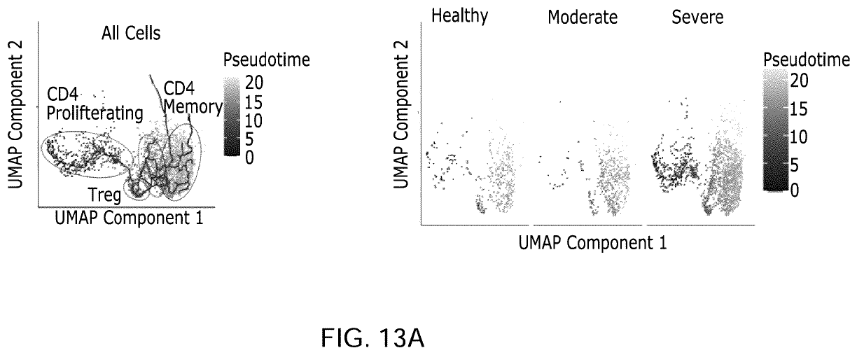

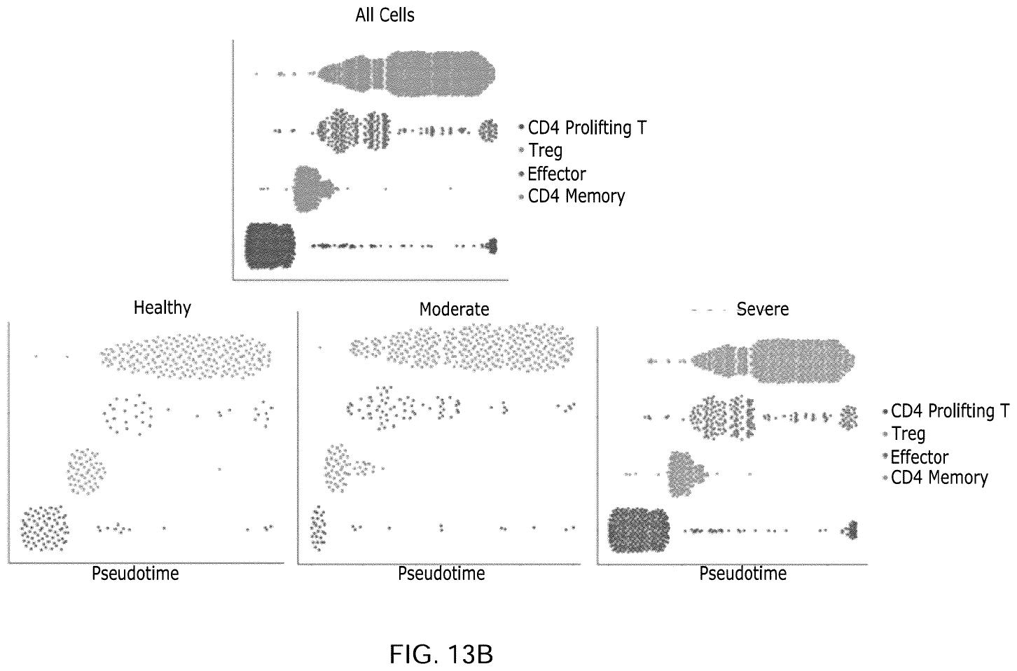

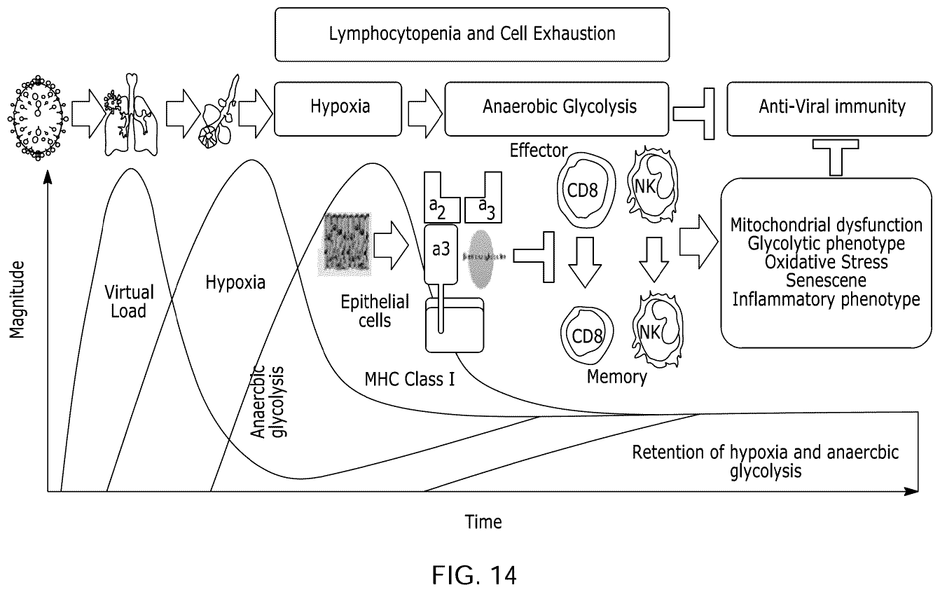

Overall, it was shown that dysregulated metabolism impairs proliferating and effector CD8 cell survival and differentiation, resulting in attenuation of antigen-specific T cell response against SARS-CoV-2 (FIGS. 3A-H). Dysfunction of CTLs and CD8 memory cells impairs terminal Trm formation (FIGS. 4A-D). Importantly, metabolic dysregulation in CD8 cells occurred at the proliferating stage. Hence, therapeutic interventions targeting exhaustion of proliferating T cells may rescue effector CD8 cell function in severe COVID-19 if administered at the initial phase of T cell recognition and clonal expansion. In contrast, no difference was found in the dynamics of CD4 cell differentiation through the trajectory inference and pseudotemporal ordering analyses (FIGS. 13A-B). This indicates that under metabolic stress, CD4 cells were able to retain proper memory formation and thus rescue differentiating trajectory capacity, which suggests the differences in CD8 and CD4 T cell responses.

### Metabolic Dysregulation in Lung Epithelial Cells Impairs Immune Surveillance and Increases Proinflammatory Response

Epithelial cells in BALF constitute the lining of upper respiratory airways and play a major role in facilitating oxygen transfer.⁴¹ As the main cell subset that expresses the ACE2 receptor in the respiratory system, epithelial cells serve as the direct target for cellular entry of SARS-CoV-2.⁴² While dysregulation of epithelial cell metabolism has been reported,⁴³ how epithelial metabolism might contribute to immunological dysfunction during SARS-CoV-2 is not well understood.

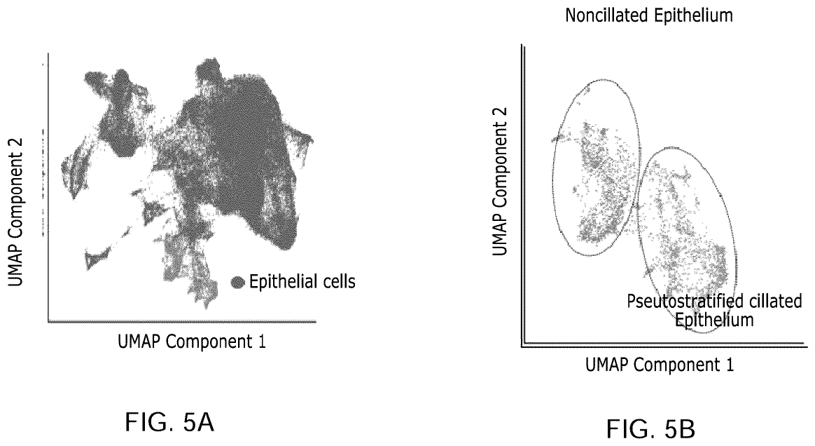

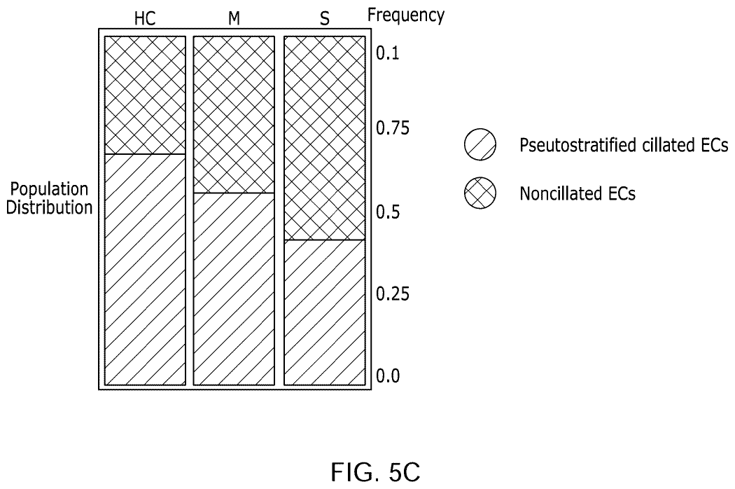

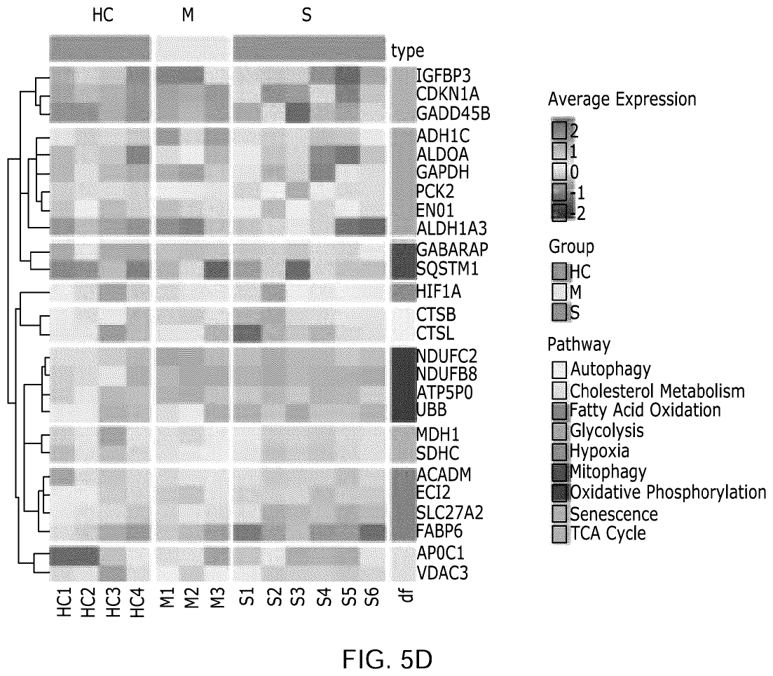

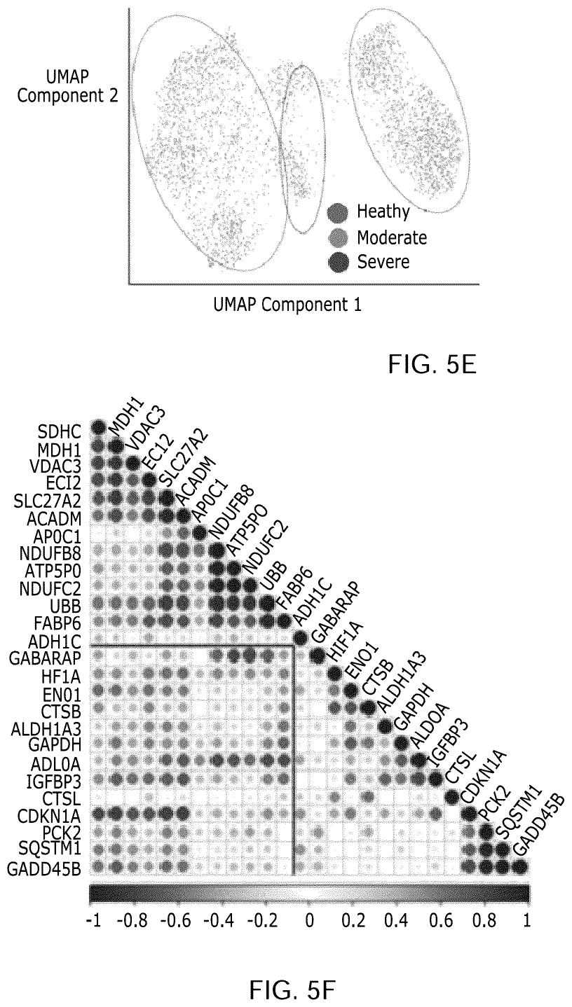

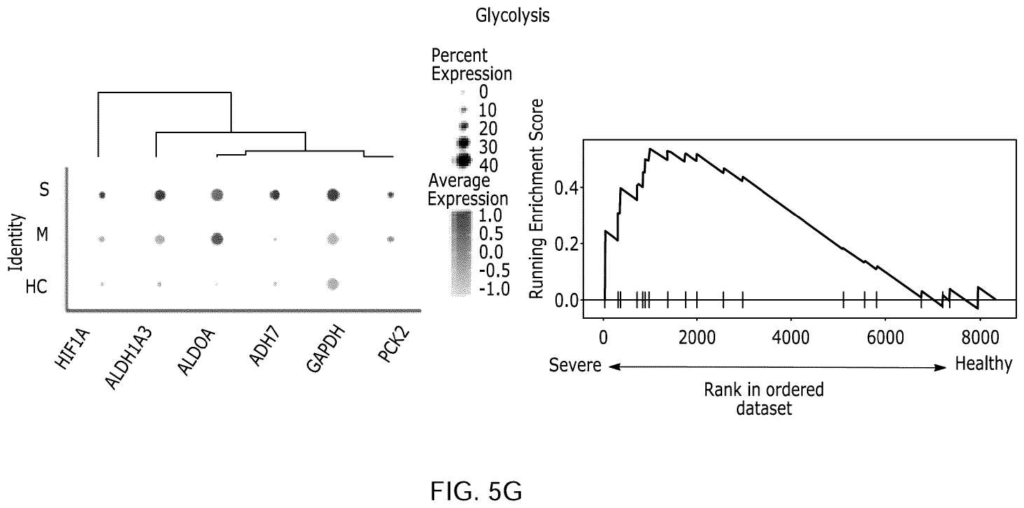

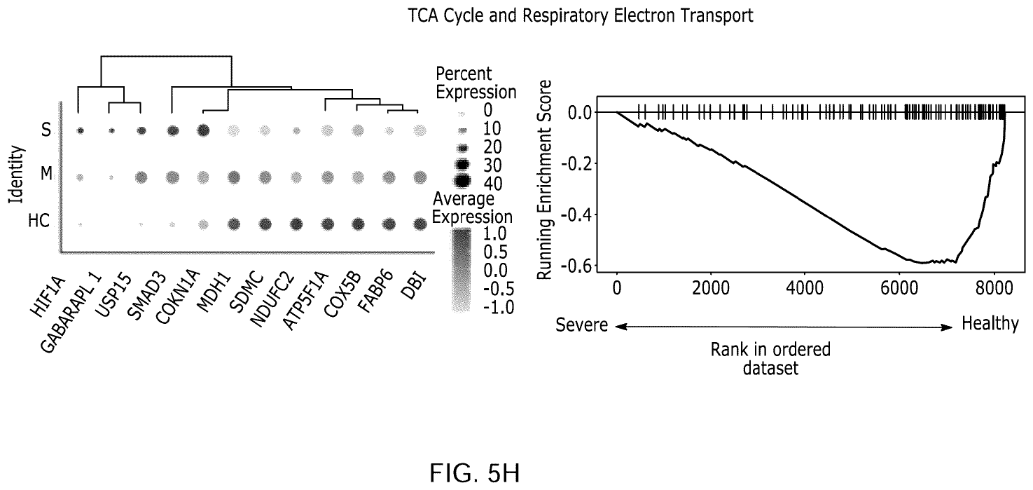

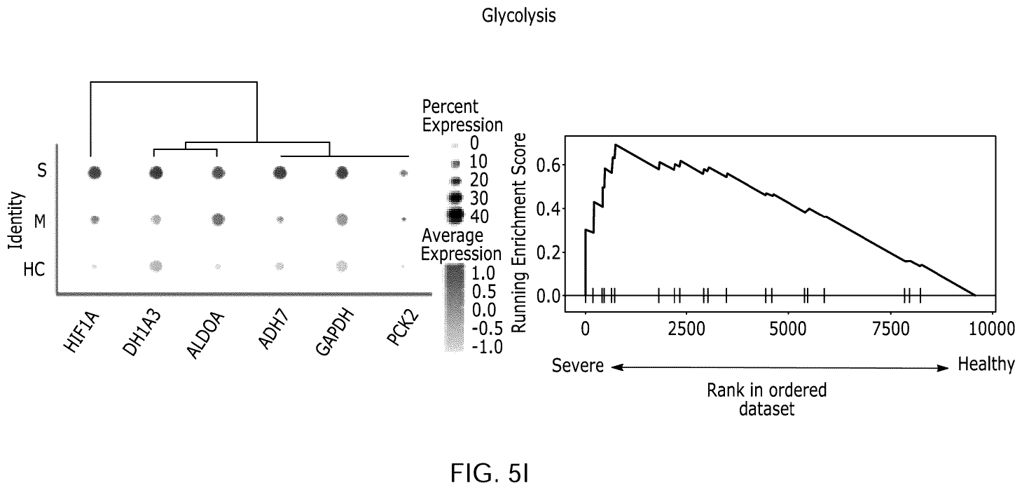

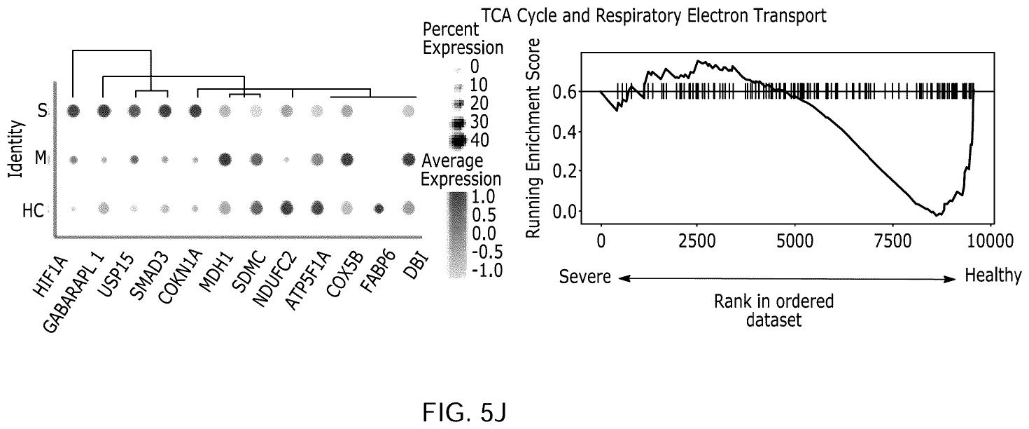

Epithelial cells were first subdivided into pseudostratified ciliated epithelial cells and non-ciliated epithelial cells based on the expression of canonical genes associated with cilia production (CFAP126, and DNAAF) (FIG. 5A). Samples HC1 and HC3 with low number of epithelial cells (<100 cells) were discarded from downstream analysis in accordance with the original study.²² The percentage of pseudostratified ciliated epithelial cells was inversely correlated with disease severity (FIGS. 1B-C), suggesting that the ciliated epithelial cell compartment was injured during SARS-CoV-2 infection.

Differential expression analysis and GSEA were then conducted using epithelial cells from severe patients and healthy controls. As expected, a remarkable increase in Hif-1α expression along with a significant decrease in OXPHOS and TCA cycle (>20 critical genes) were identified in epithelial cells from severe patients (FIG. 5B). Under conditions of oxygen deprivation conditions in the lungs of COVID-19 patients, a metabolic switch from FAO toward aerobic glycolysis by epithelial cells is indicated by decreased expression of FAO regulating genes, accompanied by marked increases in the expression of 10 key glycolytic transcripts (ADH7, PKM, LDHA, ALDHIA3, PGAM1, ENO1, GAPDH, GPI, TPI1, ALDOA). This hypoxic-induced dysregulated mitochondrial fitness is thus clearly indicated by diminished expression of genes relating to OXPHOS, TCA cycle and FAO as well as by an increased expression of genes coding for mitophagy (FIG. 5B). These factors appear to promote epithelial cell senescence as evident by increased expression of CDKN1A, GADD45B, andIDFBP3 (FIG. 5B). Moreover, increased expression of mitophagy relating genes SQSTM1, an autophagosome cargo protein, and GABARAP may in turn reflect the hyperlactatemia and hyperacidity of lung microenviroment.⁸ Pearson correlation analysis, run on pseudo-bulk data generated from averaging the expression of 26 key metabolic genes (Table 3), revealed a strong positive correlation between Hif-1α expression, senescence (CDKN1A), and mitophagy (FIG. 5C) thus validating that epithelial cell hypoxia is responsible for mitochondrial dysfunction. This metabolic reprogramming towards anaerobic glycolysis and repression of mitochondrial metabolism was present in both pseudostratified ciliated and non-ciliated epithelial cell-types, as confirmed by the analysis of differential expression profiles along with GSEA (FIGS. 5F-G).

To investigate whether the metabolic phenotype of epithelial cells can predict COVID-19 severity, UMAP and PCA were performed on a panel of 26 metabolic regulating genes (FIGS. 5D-E, and Table 3). As expected, distinct separation and clustering was found between disease conditions in plots of both UMAP and PCA components, (FIGS. 5D-E). This finding suggested that features of epithelial cell metabolism can predict COVID-19 disease severity on both a single-cell resolution and aggregate sample level.

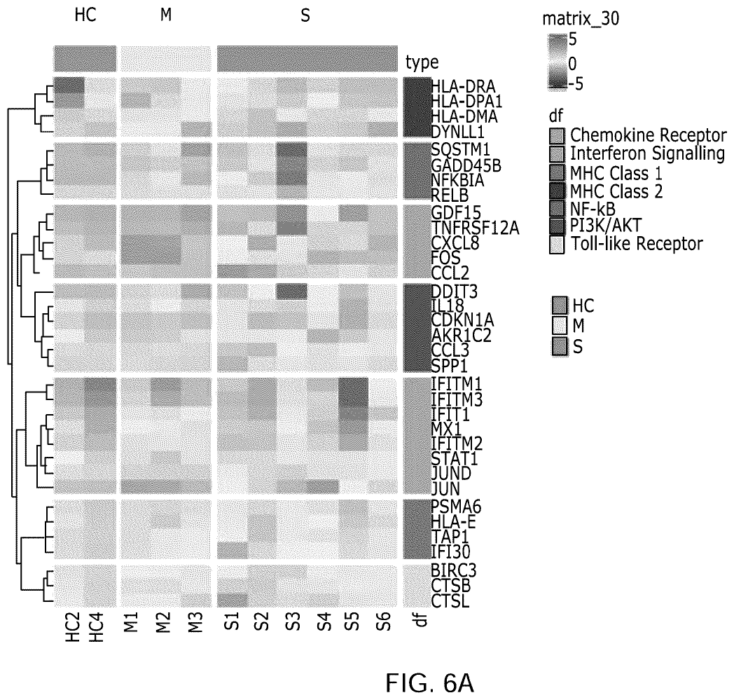

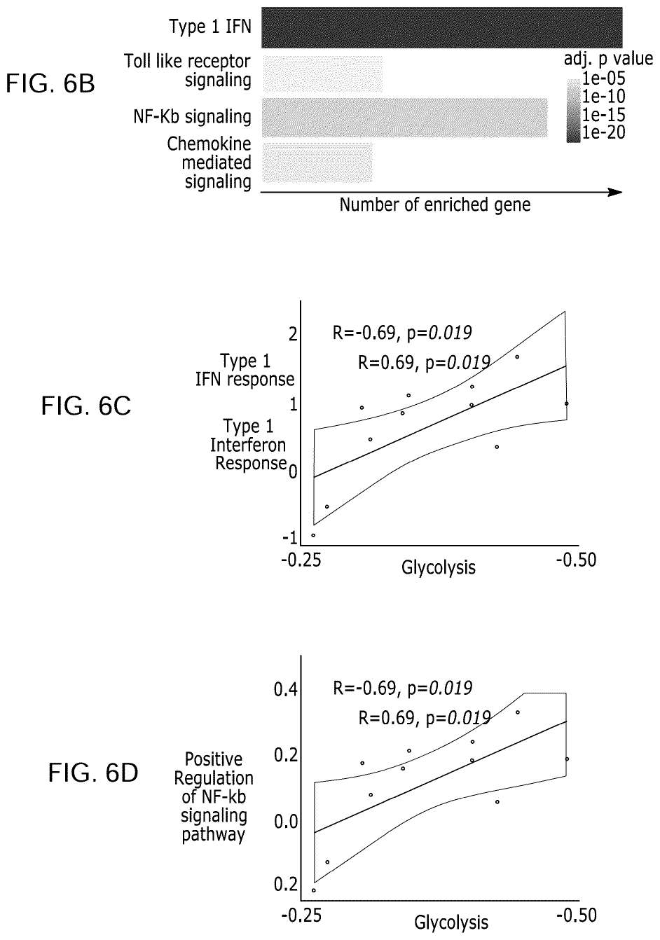

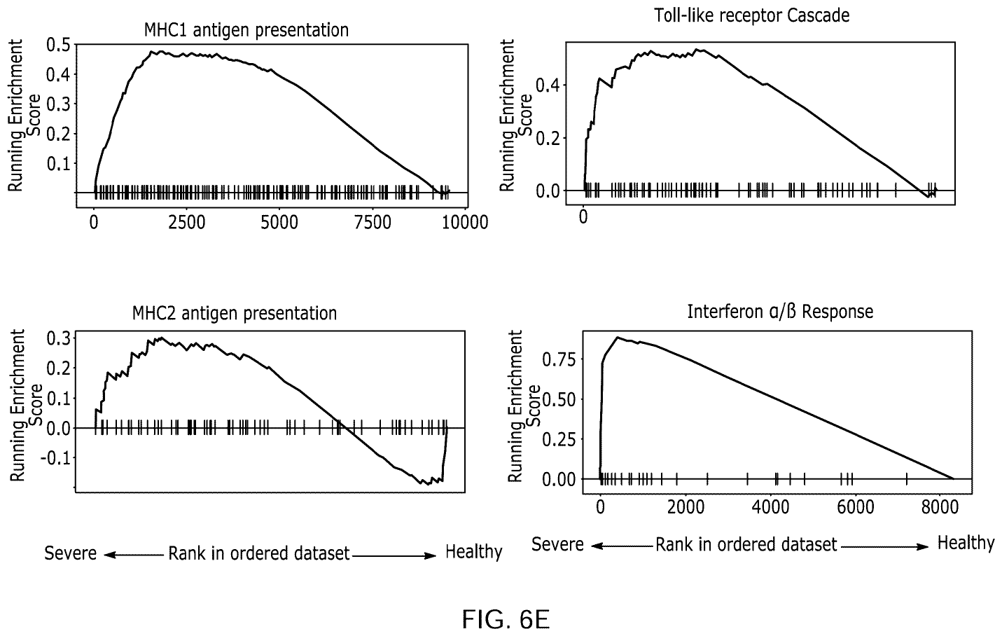

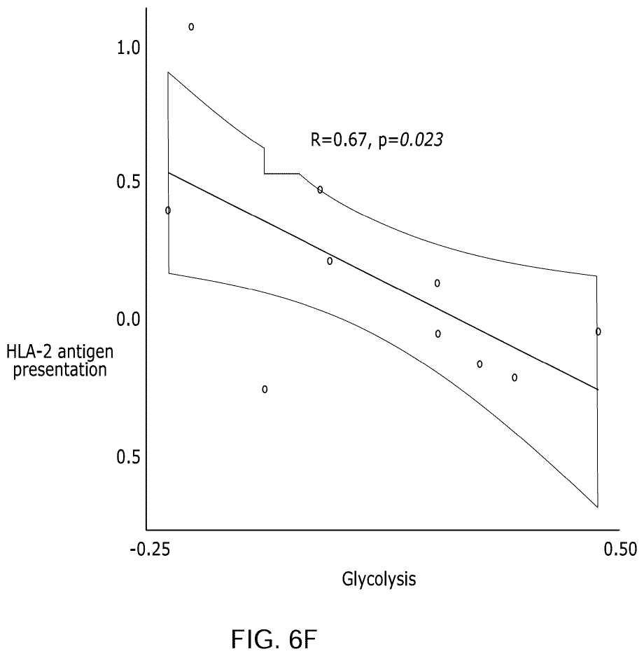

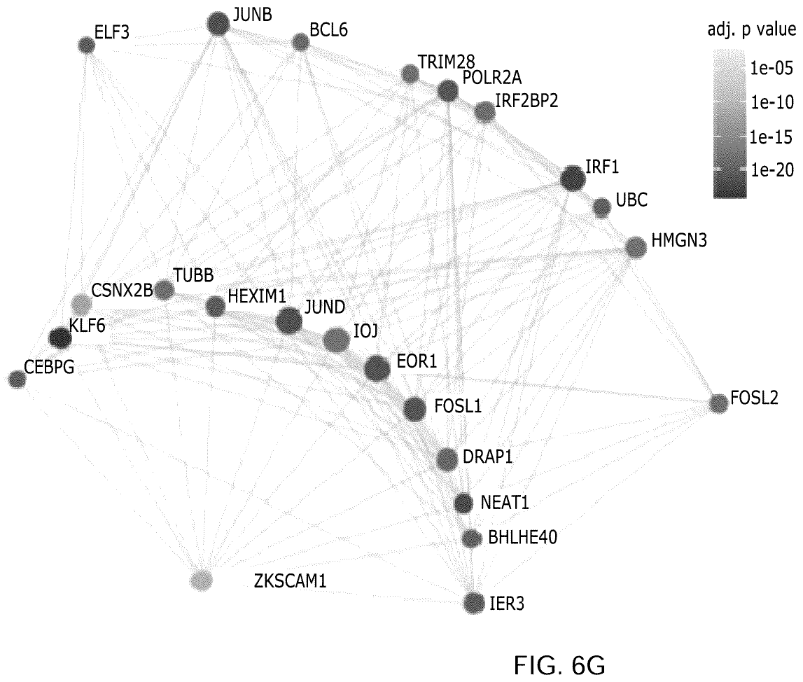

Next, how dysregulated metabolism of epithelial cells affects cytokine secretion and immune function was assessed. Pathway enrichment analysis revealed that epithelial cells acquire a senescence-associated secretory phenotype (SASP), demonstrated by overexpression of 4 key immune signaling pathways as a result of Covid-19 infection (FIGS. 6A-B). Network analysis performed on differentially expressed regulators was conducted to infer the network of transcription factor-gene interactions (FIG. 6C). It was found that negative regulation of transcription factors ZKSCAN1 and CSNK2B, and positive regulation ofKLF6, NEAT1, JUND are responsible for promoting the severe pro-inflammatory cascade induced by epithelial cells during COVID-19 (FIG. 6C). A robust increase in expression of transcripts coding for the type 1 interferon (IFN) response was observed in severe COVID-19 (FIGS. 6A-D). Increased expression of IRF1, a key transcription factor that regulates the proinflammatory epithelial cell response in COVID-19⁴⁴ (FIG. 6C), was also observed, thus indicating that DNA damage in senescent cells recruits IRF1 to induce a tumor necrosis factor (TNF)-mediating IFN response.

Chemokine receptor signaling, toll-like receptor activation and NF-kB activity were also significantly upregulated (FIGS. 6A-B). p38 mitogen activated protein kinase (p38MAPK) can be activated to upregulate NF-kB signaling. In response to DNA damage.⁴⁵ Elevated levels of lactate dehydrogenase (LDHA) (FIG. 6A), an enzyme responsible for the redirecting pyruvate from TCA cycle to lactate conversion,⁴⁶ may mediate secretion of IFN-γ and IFN-ψ through the enhancement of transcription via epigenetic acetylation. A significant positive correlation between module scores for glycolysis and type 1 IFNn signaling, as well as for glycolysis and NF-kB signaling was revealed. This suggests that hypoxia induced metabolic dysfunction is responsible for proinflammatory response of epithelial cells (FIG. 6E). Moreover, upregulation of immune signaling pathways including type 1 IFN, NF-kB, toll like receptor (TLR) and chemokine receptor activity were identified in both ciliated and non-ciliated epithelial cells (FIGS. 6A-D).

Chronic presentation of viral antigens to T cells by epithelial cells causes T cell exhaustion.⁴⁷ A significant downregulation of genes coding for HLA class 2 antigen presentation (HLA-DRA, HLA-DPA1, HLA-DMA, DYNLL1) in COVID-19-exposed epithelial cells along was observed with a moderate increase in expression of genes involving HLA Class 1 activity (HLA-E, PSMA-6, TAP1, IFI30) (FIGS. 6A-D). In addition, aerobic glycolysis was shown to repress antigen-presenting capacity,⁴⁸ which in turn, can cause T cell exhaustion and lead to impaired immuno-surveillance. Direct presentation of epithelial cells to cytotoxic CD8 cells via HLA Class 1 could result in persistent antigen stimulation of CD8 cells and thus mediate cellular dysfunction.^{49,50} Upregulation of HLA-E, a nontraditional HLA Class 1 molecule (FIG. 6A), was reported to mediate CTL exhaustion through NKG2A receptor stimulation.^{49,50} Indeed, HLA class 1 signaling is significantly upregulated during severe SARS-CoV-2 infection (FIGS. 6A and D). These findings shed light on the prevalence of lymphopenia selectively in CD8 but not in CD4 cells. GSEA analysis revealed that this dysregulation in antigen presenting function was specifically evident in pseudostratified ciliated epithelial cells (FIG. 6D).

It was shown herein that cellular metabolism in BALF epithelial cells is altered during COVID-19, leading to increased proinflammatory cytokine secretion and decreased HLA class 2-mediated immune surveillance. CD8 lymphocytopenia may be associated with augmented HLA class 1 activity. Pulmonary surfactant was found to be markedly diminished in patients with severe COVID-19.⁵¹ Therefore, reduced surfactant production resulting from direct ciliated epithelial cell damage caused by SARS-CoV-2 may explain the hypoxia-mediated mitochondrial dysfunction within the lung microenvironment during COVID-19. Given that CRS is a hallmark symptom of COVID-19 and significantly contributes to disease severity⁵² these results suggest that epithelial cell metabolism may also be responsible for proinflammatory cytokine syndrome in COVID-19.

TABLE 4

| | | | HLA Class 2 | Type 1 | |
|---|---|---|---|---|---|
| | Glycolysis | FAO | Signaling | Interferon | NfKB |
| | Module Score | Module Score | Module Score | Module Score | Module Score |
| CD8 | GAPDH, | OLR, | | | |
| Memory | GALM, GPI, | MARCO, | | | |
| | ALDOA | FABP4 | | | |
| Epithelial | ADH1C, | ACADM, | HLA-DRA, | IFITM1, | SQSTM1, |
| | ALDOA, | ECI2, | HLA-DPA1, | IFITM2, | GADD45B, |
| | GAPDH, | SLC27A2, | HLA-DMA, | IFIT1, | NFKBIA, |
| | PCK2, ENO1, | FABP6 | DYNLL1 | MX1, | RELB |
| | ALDH1A3 | | | IFITM2 | |

### Aberrant Metabolism Decreases Cytotoxicity and Promotes NKT Cell Exhaustion.

NKT cells, intermediate between CD8 and NK lineages, are critical during viral infection due to their effector function and cytokine secretion.⁵³ NKT cells play important roles in preventing pneumonia during chronic pulmonary disease.⁵³ The instant inventor and other groups observed a clear reduction of both circulating and BALF NKT cells in severe COVID-19 patients (FIGS. 1E-F), however, the mechanism for this observation has yet to be elucidated.

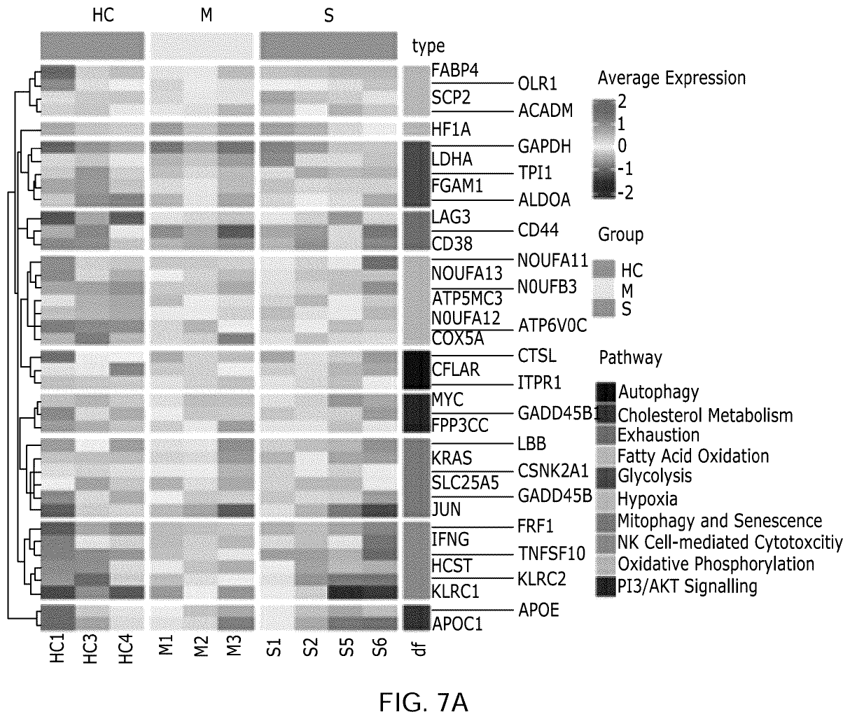

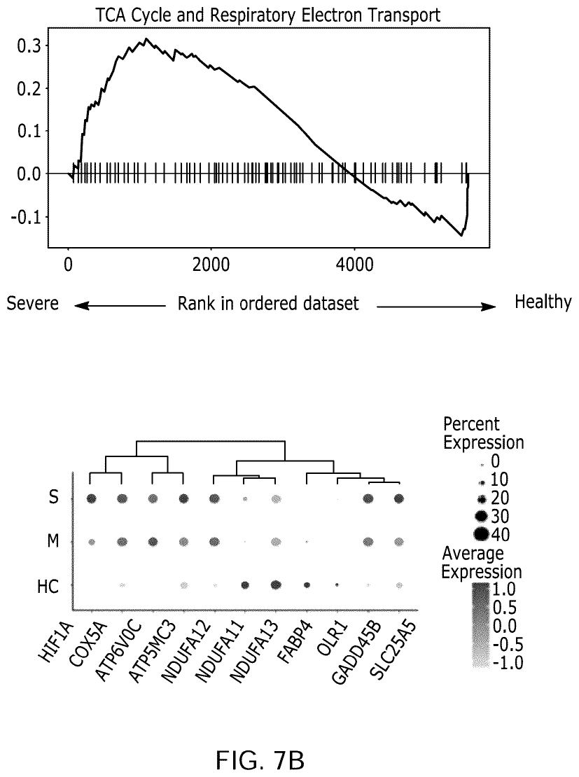

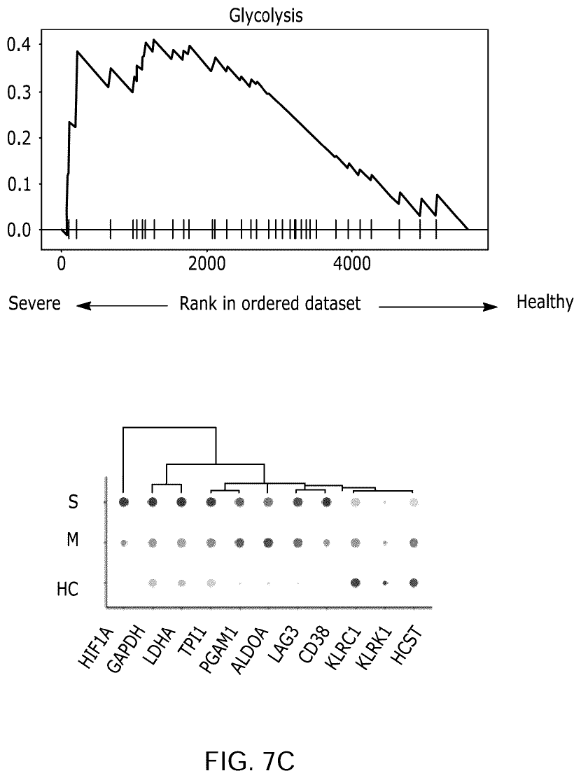

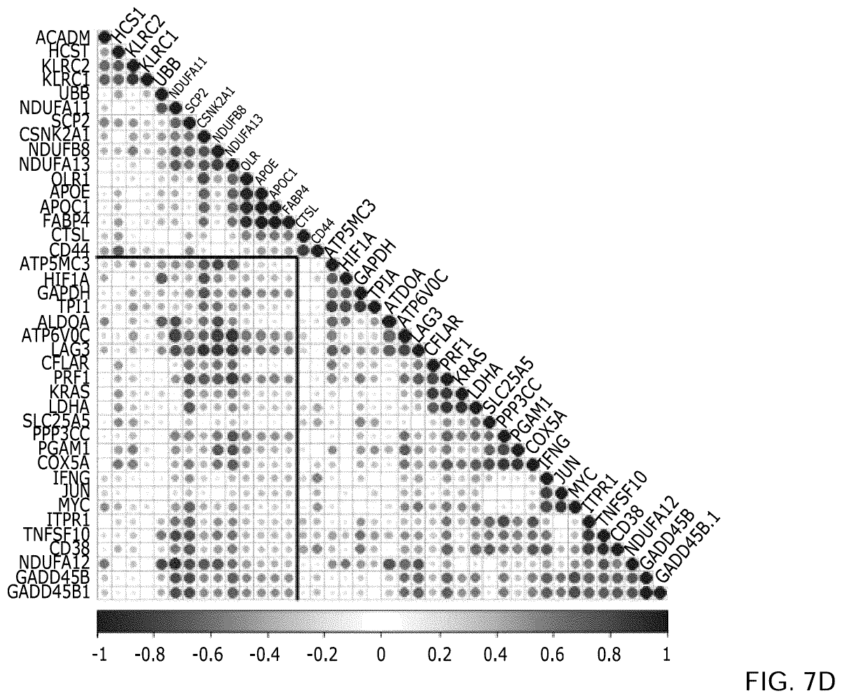

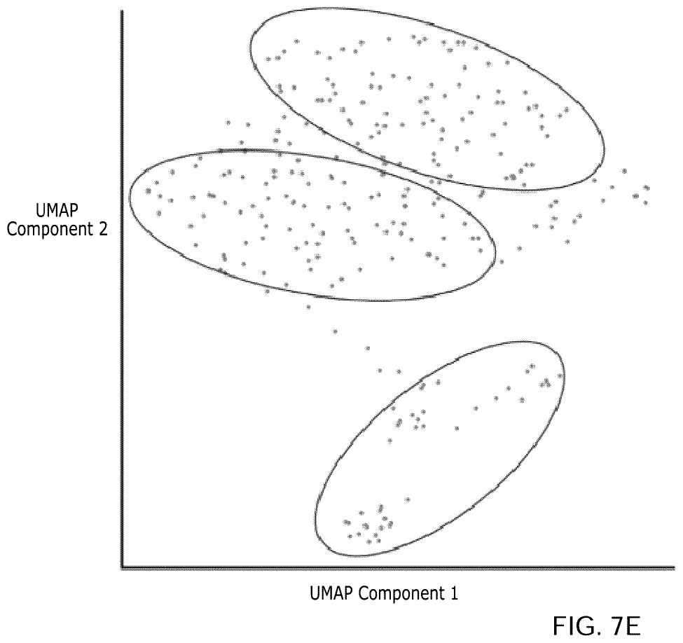

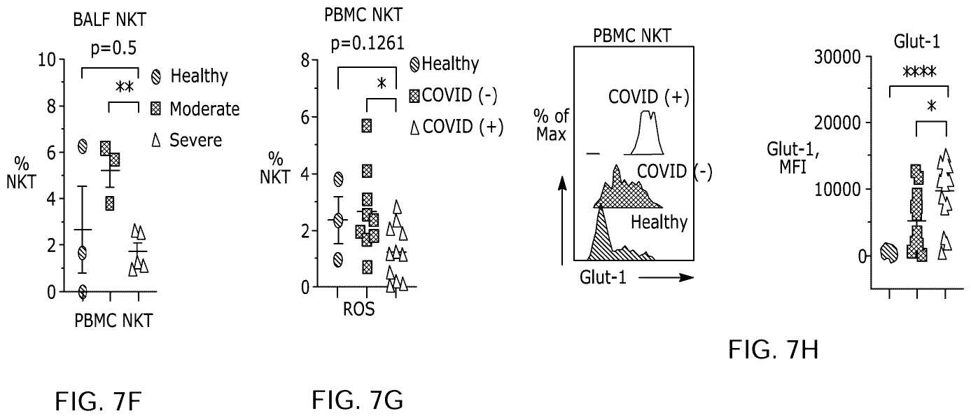

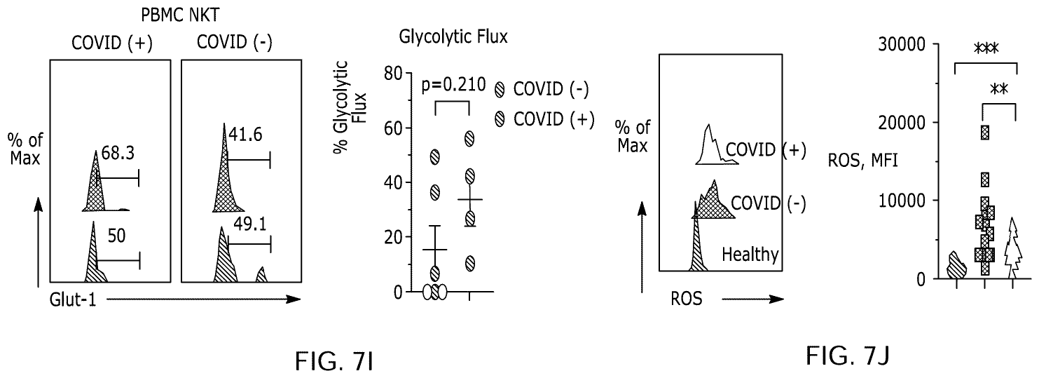

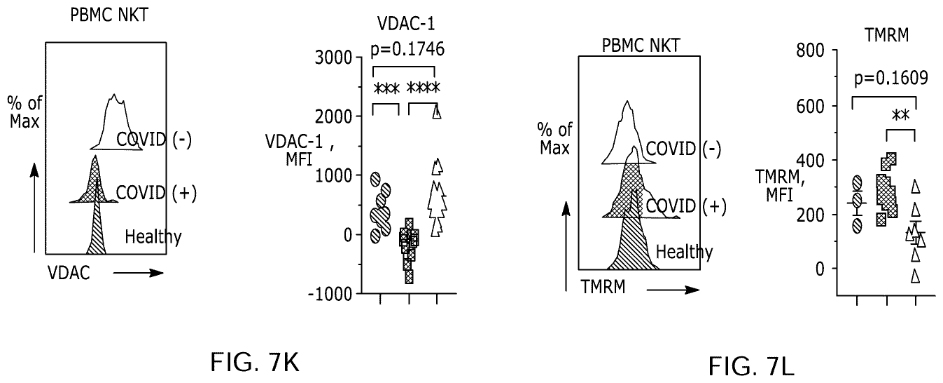

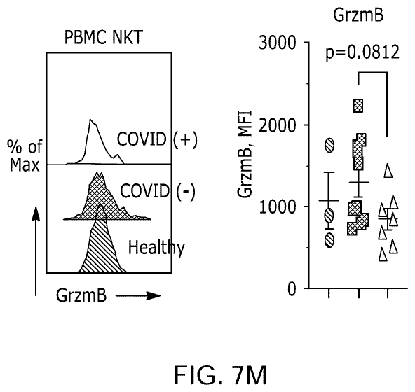

First, the immunometabolic profile of BALF NKTs in COVID-19 was characterized. Co-expression of CD8A and KLRD1 was used to define NKT cell lineage (FIG. 11A). A transcriptional program associated with hypoxia-induced metabolic shift was found in severe COVID-19 (FIG. 7A). Normally, upon activation, NKT cells utilize pyruvate dehydrogenase (PDHA1/2) which mediates pyruvate oxidation to supply acetyl-CoA to the TCA cycle to increase OXPHOS activity.⁵⁴ However, in response to the oxygen-deprived microenvironment in the lungs of COVID-19 patients, downregulation of genes coding for NADH oxidation (NDUFB8, NDUFA11, NDUFA13), along with genes involved in FAO and lipid uptake, were observed (FIGS. 7A-C). Consequently, NKT were dependent on glycolytic activity to satisfy their bioenergetic requirements (FIGS. 7A-C). Interestingly, expression of genes regulating OXPHOS and TCA cycle were significantly upregulated, suggesting that mitochondrial fitness was likely not affected by COVID-19 severity in NKT cells (FIGS. 7A-B). This observation further indicated that OXPHOS was not sufficient to support NKT effector function under hypoxic conditions. Alternatively, increased presence of GADD45B and SLC25A5 transcripts indicate the adaptation of NKT cells through the enhancement of mitophagic activity to produce basal catabolic intermediates required for effector cytokine secretion (FIG. 7A).

Next, the impact of the metabolic state on the cytotoxic function of NKT cells was investigated. As mentioned previously, epithelial cells in the BALF preferentially increase HLA Class 1 signaling and ablate HLA Class 2 machinery in response to SARS-CoV-2 infection (FIG. 6D). Because NKT cells are only restricted in response to stimulation by nontraditional HLA class 1 molecules,⁵⁵ upregulation of epithelial HLA Class 1 signaling may thus result in chronic stimulation, which leads to metabolic dysfunction and exhaustion of NKTs. Consistently, expression of canonical exhaustion markers LAG3 and CD38 was markedly increased in NKT of patients with severe COVID-19 (FIG. 7A). Furthermore, the KLRK1-HCST axis, which plays a significant role in NKT activation and cytotoxicity,⁵⁵ was significantly suppressed in severe SARS-CoV-2 infection (FIG. 7A). Expression of KLRC2, coding for NKG2C receptor expressed on mature memory cells,⁵⁶ was also reduced (FIG. 7A), indicating that impairment in memory cell formation may contribute towards NKT cell exhaustion.

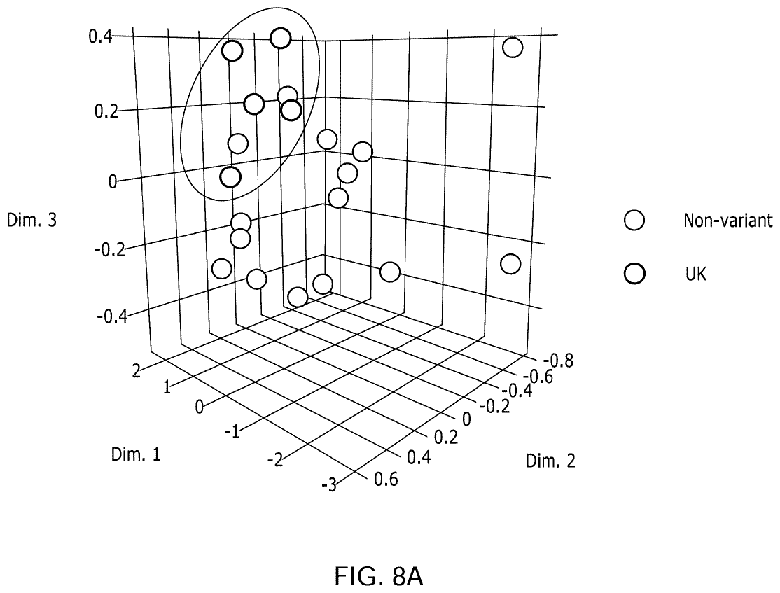

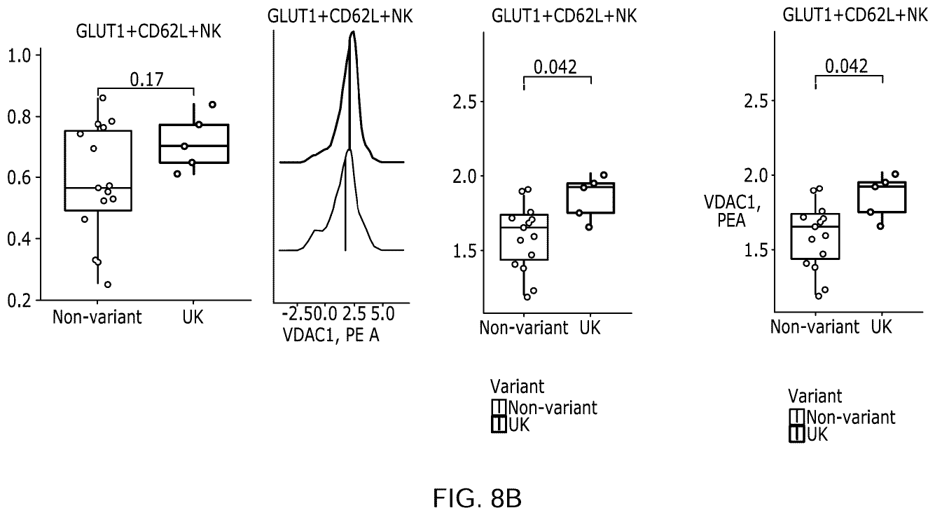

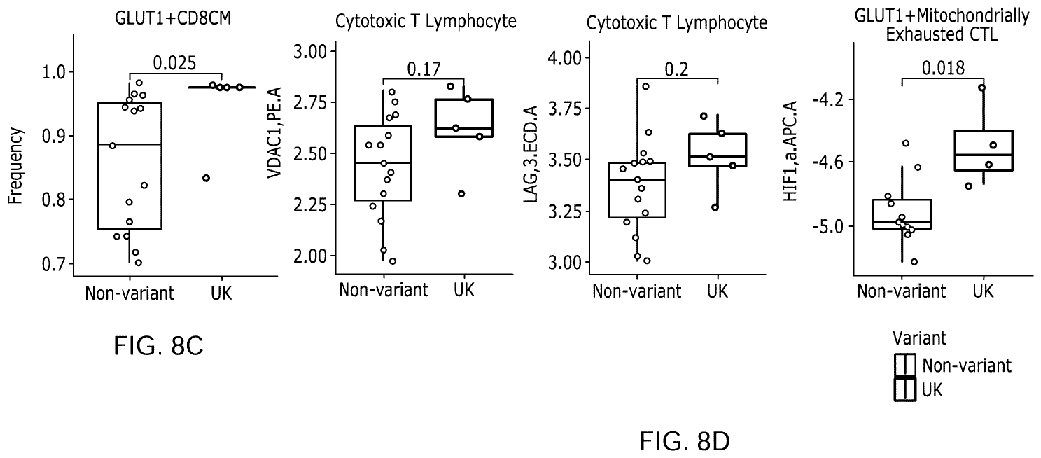

Next, the metabolic phenotype of NKTs in PBMCs from COVID-19 patients was validated. As shown in FIG. 11 and FIG. 11B, NKT cells were identified and isolated by coexpression of CD56 and CD8. There was a significantly decreased abundance of NKT cells in COVID-19 compared to healthy patients (FIGS. 1J-L). Among NKT cells, the frequency of cells with high expression of GLUT1 was largely elevated, indicating a glycolytic phenotype (FIG. 7F). Additionally, albeit not statistically significant, the number of patients with high abundance of ROS⁺ was heavily upregulated (FIG. 7F). Unsupervised clustering identified the presence of GLUT1⁺ mitochondrially exhausted cells that were positive for ROS, LAG-3, and VDAC1, which alt differentially abundant in a few select COVID-19 patients (FIG. 8B and FIG. 11F). Hif-1α expression in these patients was drastically increased in this subset of mitochondrially-exhausted cells (FIG. 8H). These data suggest that NKT cells acquire significant mitochondrial dysfunction and glycolytic phenotype in hypoxic microenvironments.

Additionally, metabolic dysregulation on a newly identified CD62L⁺NK cell population was also observed (FIGS. 6I-H). CD62L⁺NK cells were reported to behave in a memory like fashion, as they can respond to secondary activation and re-differentiate into effector subtypes.⁵⁷ Interestingly, a significant decrease in the percentage of CD62L⁺ NK cells along with an increase in NK cells were found in COVID-19 patients, (FIGS. 1J-L). This is suggestive of impaired differentiation of NK cells into their memory subtype. CD62L⁺NK cells in COVID-19 were also glycolytic as indicated by high expression of GLUT1 (FIG. 7I). Hif-1α expression was also upregulated, validating highly hypoxic conditions (FIG. 7J). A positive correlation (R=0.76, p=0.0042) of GLUT1+CD62L7 NKT percentage and serum glucose level (FIG. 7H) suggested that during severe COVID-19, individuals with a higher glucose level would exhibit metabolic reprogramming of CD62L7 NK cells toward anaerobic glycolysis, resulting in increased lymphopenia and impaired NK-mediated antiviral response.

Whether the metabolism of NKT cells can be a predictor of COVID-19 disease severity was also examined. UMAP performed for the panel of 34 key metabolic genes revealed distinct clustering between healthy, moderate, and severe samples (FIG. 7E and Table 3). Given that the unsupervised approach demonstrated clear separation between the different groups based upon metabolic features alone (FIG. 7E) suggests that the metabolic phenotype of NKT cells may potentially serve as a potential predictive biomarker for COVID-19.

Overall, it was found that under hypoxic microenvironment conditions, NKT and CD62L⁺NK cells metabolically reprogram to increase their reliance on anaerobic glycolysis and mitophagy to sustain effector function, while also maintaining OXPHOS commitment. Chronic epithelial HLA class 1 stimulation may drive NKT cells to become functionally exhausted, induce a loss of cytotoxic function, and impair memory formation.

### Distinct Immunometabolic Landscape in B.1.1.7 Variant-Infected Patients.

Recently, there has been a large surge in the prevalence of SARS-CoV-2 variant strands⁵⁸ with a predominance of the B.1.1.7 (UK) variant SARS-CoV-2 strand which is more contagious, increases risk of death. The metabolism of immune cells from B.1.1.7 variant-infected patients has not been understood.

PCA ran on averaged expression for GLUT1, ROS, LAG-3, Hif-1α, and VDAC1 indicated that CD62L⁺NK cells from B.1.1.7 variant-infected patients are metabolically distinct from non-variant (FIG. 8A). The percentage of GLUT1⁺ CD62L⁺ NK cells are moderately, albeit not statistically significant, increased in B.1.1.7 variant-infected patients (FIG. 8B), suggesting higher glucose metabolism in CD62L7 NK during B.1.1.7 variant infection. Consistently, GLUT1⁺D62L⁺NK cells have increased mitochondrial dysfunction as indicated by remarkable upregulation of VDACT (FIG. 8B). Likewise, proportion of GLUT1⁺ cells in CD8CM were also significantly elevated (FIG. 8C). Presumably, these results indicated that NK and CD8 memory cells from B.1.1.7 variant-infected patients are more dependent on glycolysis, which may lead to mitochondrial dysfunction. Furthermore, it was observed that CTLs from B.1.1.7 variant-infected patient demonstrated exacerbated immune dysregulation. Because of mitochondrial dysfunction, NK and CD8 in B.1.1.7 infected patients' cells are more functionally exhausted which is indicated by significantly increased LAG-3 expression (FIG. 8D). The level of Hif-1α was significantly elevated in a detected population of mitochondrially exhausted CTLs (FIG. 8D), indicating that hypoxia is implicated in the difference of immunometabolism in CD8 T cells from B.1.1.7 variant and non-variant SARS-CoV-2 infected patients.

Overall, these results indicate a distinct metabolic profile of PBMCs from B.1.1.7 variant-as compared to non-variant infected patients. Given CD8 and NK memory cell differentiation and function are further impaired in B.1.1.7 variant-infected individuals, raising the possibility that these individuals may be more susceptible to reinfection.

B) Detailed Description of Figures from Experiment One

FIGS. 1A-L. High-dimensional immunophenotyping revealed highly resolved single cell landscape of COVID-19 BALF and PBMCs. A) UMAP projection displaying unsupervised clusters and annotated populations of 66,452 cells from Healthy Control (4), Moderate (3), Severe (6) patients; B) Average proportion of key cell-types for each disease state; C) Box-plot of cell-type proportion for each disease state, dot represents individual sample, 2-sided Wilcoxon Mann Whitney test was performed to indicate statistical significance; D) UMAP projection displaying unsupervised clusters and annotated populations of 7601 reintegrated T cells from Healthy Control (3), Moderate (3), Severe (5); E) Average Proportion of T-cell subtype for each disease state; F) Box-plot of T cell subtype for each disease state, dot represents individual sample, 2-sided Wilcoxon Mann Whitney test was performed to indicate statistical significance; G) 3-D PCA analysis conducted using bulk expression of each marker per sample as input, circles were manually drawn around the PCA plot to highlight distinct clustering; H) UMAP projection of labelled PBMC populations from Healthy (8) and COVID-19 (20) patients; I) UMAP projections of PBMCs overlayed with scaled expression of markers used for unsupervised clustering; J) Average proportion of cell-type for each disease state; K) Box-plot of cell-type proportion for each disease state, dot represents individual sample, 2-sided Wilcoxon Mann Whitney test was performed to indicate statistical significance; and L) Contour plot of kernel density for UMAP projection of PBMCs, 5,000 cells from each condition (Healthy and COVID(+)) were randomly subsetted for density analysis.

FIGS. 2A-I. Metabolic profile of CTLs in COVID-19. A) Heatmap displaying differentially expressed metabolic genes in CTLs from the BALF; B) Dot plots demonstrating expression and hierarchical clustering of select key metabolic genes; C) GSEA enrichment plots for “Glycolysis” and “TCA and Respiratory Electron Transport” pathways comparing severe vs healthy control patients; D) Violin plot demonstrating differential distribution of expression of oxidative stress genes (NFE2L2 and PRDX2) across conditions; E) Boxplots displaying MFI values of GLUT1 in CTLs and displaying frequency of GLUT1CTLs across conditions, dot represents individual sample, 2-sided Wilcoxon Mann Whitney test was performed to indicate statistical significance; F) Boxplot displaying frequency of ROS⁺ CTLs across conditions, dot represents individual sample, 2-sided Wilcoxon Mann Whitney test was performed to indicate statistical significance; G) Density plots displaying distribution of marker expression (ROS, VDAC1, and LAG-3) for GLUT1 CTLs for all cells in each condition. Adjacent to boxplots demonstrating MFI values of markers for each sample across condition, dot represents individual sample, 2-sided Wilcoxon Mann Whitney test was performed to indicate statistical significance; H) Heatmap displaying canonical expression of labelled populations from second round of unsupervised clustering; and I) Boxplot displaying frequency of GLUT1⁺ mitochondrially exhausted cells across conditions, dot represents individual sample, 2-sided Wilcoxon Mann Whitney test was performed to determine statistical significance.

FIGS. 3A-L. Metabolic profile of CD8 Memory Cells in COVID-19. A) Heatmap displaying expression of key differentially expressed metabolic genes for CD8 memory cells; B) GSEA enrichment plots for “Glycolysis” and “TCA and Respiratory Electron Transport” pathways comparing severe vs healthy control patients; C) Linear regression and Pearson correlation analysis between module scores for Glycolysis and Exhaustion, and module scores for glycolysis and fatty acid oxidation; D) Correlation matrix showing correlation between differentially expressed metabolic genes; E) UMAP projections of CD8 memory cells clustered solely on the expression of 42 differentially expressed metabolic genes; F) Boxplot displaying frequency of GLUT1⁺ CTLs across conditions, dot represents individual sample; G) Boxplots displaying MFI values of VDAC1, Hif-1α, and LAG-3 in GLUT1+CD8EMs; H) Boxplot displaying frequency of GLUT1⁺ mitochondrially exhausted CD8EMs across conditions, dot represents individual sample; I) Boxplot displaying frequency of ROS⁺CD8TMs across conditions, dot represents individual sample; J) Boxplot displaying frequency of GLUT1⁺ mitochondrially exhausted CD8TMs across conditions, dot represents individual sample; K) Boxplots displaying frequency of GLUT1⁺ and ROS⁺CD8CMs across conditions, dot represents individual sample; and L) Boxplot displaying frequency of GLUT1⁺ mitochondrially exhausted CD8CMs across conditions, dot represents individual sample.

FIGS. 4A-D. COVID-19 induced metabolic dysfunction impairs memory formation and cellular differentiation of CD8 T-cells. A) UMAP projection of 3,694 CD8 cells from all reintegrated samples, healthy samples alone, moderate samples alone, and severe samples alone, with trajectory mappings colored by pseudotime; B) Dot plot showing pseudotime values for CD8 cells from all reintegrated samples, healthy samples alone, moderate samples alone, and severe samples alone, each dot represents a cell; C) Bar graph displaying frequency of CD8 subpopulations across disease conditions; and D) Violin plot of expression of tissue resident memory phenotype genes (ITGA1, ZNF683) in CD8 memory cells compared across disease states.

FIGS. 5A-G. Metabolic disorder of epithelial cells in COVID-19. A) UMAP projection of 3531 epithelial cells from Healthy Control (2), Moderate (3), and Severe (6) patients; B) Heatmap displaying expression of key differentially expressed metabolic genes; C) Correlation matrix showing correlations between differentially expressed metabolic genes; D) UMAP projection of epithelial cells clustered solely on the expression of 42 differentially expressed metabolic genes; E) 3D PCA plot of pseudo-bulk data of epithelial cells clustered solely on the aggregated expression of 42 differentially expressed metabolic genes; F) Dot plots demonstrating expression and hierarchical clustering of select key metabolic genes for ciliated and nonciliated epithelial cells; and G) GSEA enrichment plots for “Glycolysis” and “TCA and Respiratory Electron Transport” pathways comparing ciliated and nonciliated epithelial cells from severe vs healthy control patients.

FIGS. 6A-E. Metabolic disorder impairs immune surveillance and inflammatory response of ECs. A) Heatmap displaying expression of key differentially expressed genes regulating immune signaling; B) Bar plot showing GSEA results of key statistically significant immune signaling pathways, x axis displays number of enriched genes per pathway, bars are colored by adjusted p. value; C) Network based display of transcription factor-gene interactions of differentially expressed genes between severe and healthy patients; D) GSEA enrichment plots for “HLA Class 2 Antigen Presentation”, “HLA Class 1 Antigen Presentation”, “Toll-like Receptor Cascade”, and “Interferon A/B Response” pathways comparing severe vs healthy control patients for pseudostratified ciliated epithelial subset; and E) Linear regression and pearson correlation analysis between module scores for glycolysis and HLA Class 2 Signaling, glycolysis and Type 1 interferon response, and glycolysis and NF-kB signaling for all epithelial cells.

FIGS. 7A-K. Aberrant metabolism of NKT and CD62L+ cells in COVID-19 patients. A) Heatmap displaying expression of key differentially expressed metabolic genes; B) GSEA enrichment plots for “Glycolysis” and “TCA and Respiratory Electron Transport” pathways comparing severe vs healthy control patients; C) Dot plots demonstrating expression and hierarchical clustering of select key metabolic genes; D) Correlation matrix showing correlations between differentially expressed metabolic genes; E) UMAP projections of NKT cells clustered solely on the expression of 34 differentially expressed metabolic genes; F) Boxplot displaying frequency of GLUT1⁺ and ROS⁺NKT cells in PBMCs across conditions, dot represents individual sample; G) Boxplot displaying frequency of GLUT1⁺ mitochondrially exhausted cells across conditions, dot represents individual sample; H) Density plots displaying distribution of marker expression of Hif-1α for GLUT1⁺ mitochondrially exhausted NKT cells for all cells in each condition. Adjacent to boxplots demonstrating MFI values of Hif-1α for each sample across condition, dot represents individual sample; I) Boxplot displaying frequency of GLUT1⁺ CD62L⁺ NK cells in PBMCs across conditions, dot represents individual sample; J) Boxplot displaying MFI values of Hif-1α of GLUT1⁺ CD62L⁺ NK cells across conditions, dot represents individual sample; and K) Scatterplot demonstrating correlation between serum glucose level and frequency of GLUT1⁺ CD62L⁺ cells in COVID-19 patients, linear regression line with error bars displayed along with spearman correlation statistics.

FIGS. 8A-D. Differential metabolism of memory cells in B.1.1.7 infected patients. A) 3D PCA plot of pseudo-bulk data of CD62L⁺ NK cells clustered solely on the aggregated expression ROS, GLUT1, Hif-1α, LAG-3, and VDAC1; B) Box-plot displaying percentage of GLUT1⁺ CD62L⁺ NK cells for non-variant and variant samples, histogram displaying VDAC1 expression in GLUT1+ CD62L⁺ NK cells, box-plot displaying MFI values for VDAC1 in GLUT1⁺ CD62L⁺ NK cells; C) Box-plot displaying percentage of GLUT1⁺ CD8 central memory cells for non-variant and variant samples; and D) Box-plot displaying MFI values for LAG-3 and VDAC1 in CTLs, box-plot displaying MFI values for Hif-1α in mitochondrially exhausted CTLs.

FIGS. 9A-B. Heatmap displaying canonical gene expression of genes used to annotate unsupervised clusters for all 66,452 cells after sample integration. FindAllMarkers function in Seurat was used to identify the top genes specific to each annotated cell population A) CD4, Macrophage, Peripheral Monocyte, Nonciliated Epithelium, B, CD8, mDC; and B) pDC, Proliferating T, Plasma, Ciliated Epithelium, Neutrophil, and Mast). A heatmap displaying the average expression of the genes for each cell subset was generated using the DoHeatmap function in Seurat.

FIG. 10. Heatmap displaying canonical gene expression of genes used to annotate unsupervised clusters after T cell reintegration. FindAllMarkers function in Seurat was used to identify the top genes specific to each annotated T cell population. The average expression of the genes for each T-cell subset was generated using the DoHeatmap function in Seurat.

FIGS. 11A-G. Heatmap displaying canonical marker expression of phenotypic markers. For unsupervised clusters generated through flowSOM (A); For annotated populations of PBMCs (B); CTLs in PBMCs (C); CD8EMs in PBMCs (D); CD8TMs in PBMCs (E); CD8CMs in PBMCs (F); and NKT cells in PBMCs (G).

FIGS. 12A-D. A) Heatmap displaying expression of key differentially expressed metabolic genes; B) GSEA enrichment plots for “Glycolysis” and “TCA and Respiratory Electron Transport” pathways comparing severe vs healthy control patients; C) Dot plots demonstrating expression and hierarchical clustering of select key metabolic genes; and D) Volcano plot of differentially expressed genes between severe and healthy COVID-19 patients, x axis shows log 2 fold change, y axis shows adj. p value.

FIGS. 13A-B. A) UMAP projection of 2603 CD4 cells from all reintegrated samples, healthy samples alone, moderate samples alone, and severe samples alone, with trajectory mappings colored by pseudotime; and B) Dot plot showing pseudotime values for CD4 cells from all reintegrated samples, healthy samples alone, moderate samples alone, and severe samples alone, each dot represents a cell.

Discussion of Results Metabolic syndrome and its constellation of deleterious effects are important risk factors for COVID-19 lethality.^{5,59} There is an urgent need for predictive biomarkers for COVID-19 severity. Understanding the role of cellular metabolism in COVID-19 pathogenesis is imperative not only for COVID-19 prognosis, but also to help clinicians choose the best therapeutic approach. The instant invention used high dimensional analytical approaches to construct a comprehensive metabolic landscape of immune cells participating in the anti-viral response against SARS-CoV-2. Evaluating cells from both the BALF and the blood of hospitalized COVID-19 patients unveiled a global metabolic adaptation in key lymphocyte populations. The instant inventor also identified novel immune cell subsets exhibiting metabolic dysfunction that could serve as predictive biomarkers for COVID-19 severity.

Despite the fact that metabolism dictates the fate and function of immune cells during viral infection including COVID-19,⁶⁰ prior attempts to elucidate the role of metabolism in lymphocyte exhaustion during COVID-19 are limited to analysis of patient-derived PBMCs.^{8,39,61} ARDS is the main contributor to mortality and severe complications during COVID-19 infection.⁶² Immunometabolic profiles of circulating blood lymphocytes under cryogenic preservation or ex vivo normoxic conditions may not adequately reflect and account for severity of infection.⁶³ Thus, in the described study, analyses on immune cells from BALF of severe COVID-19 patients and freshly isolated PBMCs from hospitalized COVID-19 patients²⁰ were performed.

Lymphocytes, including CD8 and NK cells, are the key drivers of antiviral immunity.^{23,64} Lymphocytopenia, a reduction in lymphocyte abundance associated with cellular exhaustion, was well documented in patients with severe COVID-19.^{65,66} Accordingly, mitigating lymphocyte exhaustion is as an attractive therapeutic strategy COVID-19.^{59,65} Herein, it was observed that under hypoxic conditions, there is a metabolic reprogramming of lymphocyte subsets traditionally reliant on OXPHOS and FAO toward anaerobic glucose metabolism, which along with mitochondrial dysfunction, triggers cellular exhaustion, resulting in a compromised anti-viral response. Cossarizza et al. used flow cytometry and metabolic flux assays to characterize the immunometabolic phenotype of isolated PBMCs from COVID-19 patients.⁶¹ An increased abundance of exhausted PD1⁺ lymphocytes in severe patients was not accompanied by any difference in extracellular acidification (ECAR) and oxygen consumption rates (OXPHOS).⁶¹ The Powell group discovered a novel population of VDAC1⁺ exhausted T cells in PBMCs from severe COVID-19 patients using flow cytometry and single cell RNA-sequencing.⁶⁷ However, they could not detect any significant change in the expression of glycolysis-regulating genes in T cells,⁶⁷ which is contradictory to the results presented herein. One potential explanation for this discrepancy is that the aforementioned study stimulated isolated PBMCs with αCD3/CD28 polyclonal activation or with SARS-CoV-2 specific peptide libraries. Prolonged maintenance and stimulation of T cells under normoxic conditions is not reflective of the hypoxic microenvironment conditions present during severe COVID-19 infection, which may result in the attenuation or nulling of any potential metabolic differences present in COVID-19. Another potential explanation arises from the fact that cellular metabolism in these studies was assessed for entire T cell populations. Given that T cells are highly heterogeneous with respect to their metabolism, metabolic profiles need to be assessed on specific T cell subpopulations instead of bulk T cells. In contrast, by using high-dimensional flow cytometry to investigate the single-cell metabolism of unstimulated PBMCs immediately after isolation, and assessing single cell transcriptomics data from the BALF, the metabolic state of the cells from their original microenvironment was captured. The single-cell omics approach allowed detection of distinct differences in metabolic phenotypes in specific, highly resolved lymphocyte populations. It was found that in response to a metabolic shift of CD8 cells to anaerobic glycolysis, CTLs could not effectively transit into memory phenotype and under persistent HLA class 1 antigen stimulation from epithelial cells, became functionally exhausted. Therefore, targeting T cell glycolysis during COVID-19 infection might be a promising approach for rescuing T cell fate and function. Ideally, it is suggested that attempts to target T cell glycolysis in COVID-19 should take place after clonal expansion and formation of initial antigen specific T cells, but before initiation of memory cell formation. It is possible that the efficacy of dexamethasone treatment in COVID-19 patients on mechanical ventilation and supplemental oxygen⁶⁸ may be due to steroid-mediated inhibition of glycolysis.⁶⁹ Thus, dexamethasone administration may be able to rescue T cell dysfunction, improve memory cell formation, and reduce CRS via inhibition of glycolysis.⁷⁰ In support of the importance of time-dependent treatment for COVID-19, type 1 IFN, which was reported to induce the metabolic reprogramming from glycolysis into OXPHOS and FAO in immune cells,⁷¹ was only effective as a treatment option in COVID-19 when administrated early after infection.⁷² In contrast, delayed type 1 IFN treatment resulted in worsening of COVID-19 severity due to the pro-inflammatory induction capacity of this cytokine.⁷² Further, the results presented herein implicate mitophagy as a potential target for therapeutic intervention. In response to a nutrient-depleted and hypoxic microenvironment, effector CD8 T cells may upregulate mitophagy as an alternative survival mechanism. However, prolonged upregulation may induce lymphocyte exhaustion and mitochondrial dysfunction. In support the assumption, ablating mitophagy in CTLs can potentially redirect T cells towards memory cell differentiation and rescue them from exhaustion.⁷³ Furthermore, unlike glycolysis, mitophagy is not critical for T cell activation and effector cell differentiation.⁷³ Thus, mitophagy-targeting approaches can potentially be used immediately after COVID-19 infection. Hydroxychloroquine (HQ), an autophagy inhibitor inhibiting autophagosome-lysosome fusion,⁷⁴ was used early in the COVID-19 pandemic with only some minor reports of efficacy, but was eventually deemed ineffective in randomized clinical trials.^{75,76} It is likely that any observed efficacy is associated with its capacity of inhibiting mitophagy;⁷⁷ however, the cytotoxicity of HQ may attenuate its positive effects.⁷⁸ Targeting mitophagy with a specific mitophagy inhibitor such as Mdivi-1 should be explored as a treatment regimen for COVID-19.

Supplemental oxygen as opposed to ventilation was shown to improve the outcome of patients with severe COVID-19.⁷⁹ Mechanistically, the study described herein suggests that hypoxia is the key regulator of immunometabolic dysfunction during severe SARS-CoV-2 infection. Hence, efforts to maintain blood oxygen saturation early in the course of infection are vital for patient recovery and improvement. Immunological outcomes post-initial infection such as memory cell formation dictate the severity of response upon re-exposure to the virus.⁴⁷ In this regard, metabolism can also influence immune response after recovery from SARS-CoV-2. Because both effector function and memory differentiation are severely impaired in lymphocytes, T cell immunity may be compromised even in recovered patients. As the COVID-19 reinfection rate has been cumulatively arising,⁸⁰ future studies investigating how metabolism affects the humoral response, including activation, antibody secretion, and long-term plasma cell differentiation of memory B, are imperative. No population of T follicular helper cells in the BALF from 13 severe patients was detected, suggesting that metabolic dysregulations also impaired germinal center formation in the lungs of COVID-19 patients. In support of this observation, hypoxia and nutrient deprivation are known to suppress the generation of germinal center B cells and follicular helper cells after viral infection.⁸¹ Additionally, defective Tfh and germinal B cell formation in spleen and lymph node, along with SARS-CoV-2-specific B cell enrichment in blood of severe COVID-19 patients, were recently reported.⁸² Overactivation of extrafollicular B cells in COVID-19 is also indicative of germinal center impairment.⁸³ These findings suggest that patients with metabolic comorbidities or ARDS may suffer from limited durability of antibody responses during COVID-19 infection. Furthermore, knowledge of how cellular metabolism regulates memory cell differentiation may help predict the reaction of patients with preexisting metabolic comorbidities to vaccination. Although COVID-19 vaccines that have been approved for emergency use, their effectiveness in inducing long term immunity has yet to be established.^{84,85} Furthermore, there has yet to be an attempt to understand the longevity of convalescence-induced protective immune responses in COVID-19 patients with metabolic disorders.

Lung epithelial cells constitute a biological layer that physically prevents viral access and facilitates oxygen transport.⁴¹ Lung epithelial cells are an important barrier to SARS-CoV-2 infection. Due to the expression of ACE2, a receptor required for COVID-9 entry, epithelial cells are directly damaged by SARS-CoV-2, which in turn creates oxygen-deprived conditions in the lungs that not only induce metabolic reprogramming of various immune cell subsets, but also themselves. It was found that during COVID-19 infection, lung epithelial cells are prone to senescence and acquire a significant SASP phenotype, leading to secretion of proinflammatory cytokines, reduced HLA class 2 mediated immunosurveillance, and increased HLA class 1 machinery. Chronic stimulation of lymphocytes by antigen presenting cells suggests a novel mechanism responsible for COVID-19 lymphocytopenia relating to non-conventional antigen presenting function of epithelial cells. This observation indicates that in addition to HLA class 2 presentation hematopoietic stem cells (monocyte, DCs, and macrophage), which has been well-accepted to contribute to lymphocytopenia in COVID-19,^{86,87} HLA class 1 expressed on non-hematopoietic cells should also be considered for the therapeutic development.

Unconventional T cells such as NKTs are depleted in COVID-19.⁸⁸ However, the mechanisms underlying these observations are unknown. Herein, evidence suggesting that epithelial cells induce NKT exhaustion and dysfunction through chronic antigen stimulation resulting from a hypoxia-mediated metabolic adaptation is provided. The prevailing view that NK cells are short-lived innate lymphocytes, is being challenged by new data showing that NK cells can develop long lasting, antigen-specific memory in response to viral infection.⁸⁹ Recently, CD62L^{high} NK were identified as a subset possessing multiple characteristics of memory cells demonstrating rapid responsiveness towards viral restimulation.⁵⁷ In the current study, it was discovered that metabolic disorders cause CD62L^{high} NK lymphocytopenia via impairment of memory formation. Because the abundance of GLUT-1⁺CD62L⁺NK cells can predict the COVID-19 severity, further studies about the role of CD62L^{high}NK cells in COVID-19 pathogenesis are crucial.

Cellular glucose uptake is essential for lymphocyte activation and mediated by the glucose uptake transporter (GLUT) family. Among 14 members of GLUT family, GLUT-1 (Slc2a1) was shown to be essential for maintaining glycolytic activity in T cells.³¹ Overexpression of GLUT-1 in response to prolonged hypoxia is an adaptive mechanism⁹⁰ that is associated with proinflammatory function. In the current study, it was discovered that multiple cell subsets of CD8, NK and NKT which highly express GLUT-1 and can be used as predictive biomarkers for COVID-19 severity. It was found that increased GLUT-1 expression was accompanied by mitochondrial dysfunction, cellular exhaustion, and memory differentiation disruption. Together these findings suggest that hypoxia and anaerobic glycolysis signaling provokes metabolic disorders in COVID-19, which can lead to compromised innate and adaptive immunity against the virus.

Despite claims of biomarkers to predict COVID-19 severity⁹¹, no specific markers for COVID-19 patients with metabolic co-morbidities have been yet discovered. In the current study, using high dimensional analyses, a number of low abundance cell subsets in the blood of severe COVID-19 patients that can potentially predict disease severity including GLUT1⁺ mitochondrially exhausted CTL, CD8CM, NKT and NK cells, are provided. Noticeably, a clear correlation between the serum glucose level, recently identified as risk factor COVID-19 severity in patients with pneumonia,⁹² and GLUT-1^{high} CD62L⁺ NK cells was observed suggesting that the use of metabolic biomarkers in combination can be strong prognostic indicator for COVID-19 disease severity.

The absence of any differences in the metabolism of pDCs, mDCs, neutrophils, and traditional NKs in BALF immune cells in severe COVID-19 is notable (data not shown). Despite sharing a similar metabolic reprogramming with CD8 T cells, no cellular exhaustion and memory cell differentiation was observed in CD4 T cells. This is likely due to the lack of germinal center formation⁸² preventing the interaction of CD4 T cells with antigen presenting cells including epithelial cells, pDCs, mDCs that help to reduce the impact of metabolic dysfunction in CD4 T cells. In support of this assumption, expression of HLA class 2 was heavily downregulated in epithelial cells. Finally, macrophage and monocyte metabolism was significantly altered during severe COVID-19 infection, as reported by Moraes-Vieira et al.⁹³

With the occurrence of novel COVID-19 variants, which are more virulent and evade the existing vaccines,⁹⁴ it is vitally important to understand how trained and gained immunity differ amongst patients infected with each viral strain. Evidence from the current study indicated that immunometabolism of host can be the key factor that decides the toxicity of SARS-CoV-2 variants. Therefore, further study on immunometabolism in patients recovered from SARS-CoV-2 variants would be utmost important and urgent. Similarly, study on a vaccinated patient who is reinfected with SARS-CoV-2 variant can provide insightful evidence for the viral evade against gained or trained immunity. Finally, combining therapies of metabolism-targeting therapies with others for acute infection and as an adjuvant for vaccination should be considered in clinics.

### Experiment Two

### Introduction to Experiment Two

As previously noted, COVID-19 is one of the most severe health crises in history.¹ In most infected individuals, the host immune response is sufficient to clear the infection. However, because of insufficient and dysfunctional immune response towards SARS-CoV-2 infection, some individuals acquire severe disease marked by significant lung damage.¹¹ The growing body of evidence suggests a link between host immune cells and patient metabolism during severe COVID-19.¹⁰⁶ Prior metabolic comorbidities and their associated cluster of conditions pose are potent risk factors for disease severity.⁴⁻⁷ Patients with type 2 diabetes, obesity, hyperglycemia, dyslipidemia, and older age have higher rates of severe complications and mortality.⁶⁰ Interestingly, host metabolic rebalance using cholesterol-lowering-(statins), glucose metabolism-reducing (2-deoxyglucose), and antioxidant (melatonin) drugs have been shown to benefit COVID-19 treatment.

T cells (Tc) including CD4 and CD8 are major cell subsets providing protective immunity against SARS-CoV-2 infection.⁹ Indeed, SARS-CoV-2-induced Tc lymphopenia is evident in 83.2% of COVID-19 patients with acute respiratory distress syndrome (ARDS).¹⁰ The processes of Tc activation, differentiation, and maturation into memory and effector subsets are tightly regulated by metabolic reprogramming.” Upon TCR activation, mTOR signaling initiates a glucose transporter 1 (glut-1)-mediated increase in aerobic glycolytic flux that is required for activation.¹² Whereas the downstream differentiation of effector CD8+Tc is heavily dependent on the activity of prolonged aerobic glycolysis; increased lipid uptake and mitochondrial fatty acid oxidation (FAO) is required for memory cell differentiation.¹³ It has been shown that under hypoxic condition, hypoxia inducible factor a (Hif-1α) redirects pyruvate from mitochondrial shuttling towards lactate conversion while increasing Tc intrinsic ROS, resulting in impaired mitochondrial function and hampered memory cell differentiation.⁵ While the accumulation of ROS was found in Tc from COVID-19 patients with ADRS¹⁴ it is completely unknown how condition of oxygen deprivation would affect the balance of effector and memory Tc phenotypes as well as the abundance of viral antigen-specific Tc during COVID-19. Moreover, it is also unclear whether Tc fate and function would be different in severe COVID-19 compared to other lung pathologies.

Mitophagy is a cellular process involved in selective degradation of damaged mitochondria.¹⁵ During conditions of hypoxia, cells upregulate mitophagy to direct their metabolism towards aerobic glycolysis as a mechanism to maintain their survival.¹⁶ While enhancing mitochondrial damage has been identified in Tc from COVID-19 patients with ADRS,⁵ the role of mitophagy in regulating mitochondrial functions during SARS-CoV-2 infection remains unexplored. Importantly, inhibition of mitophagy has been shown to inhibit viral proliferation in virally infected cells; thus, suggesting that mitophagy-targeting strategies may have a combinatorial effect of altering host immunometabolism as well as directly decreasing viral load.¹⁷

The abundance of CD56⁺CD8⁺ cells (NKT) is a strong predictive biomarker for COVID-19 outcome.¹⁸ It has been apparent that NKT play an important role in the prevention of COVID-19-induced pneumonia.¹⁹ As a key bridge between innate and adaptive immunity,²⁰ little is known about how NKT metabolism during SARS-CoV-2 infection. Interestingly, in contrast to Tc, NKTs are considerably more dependent on mitochondrial metabolism after activation.^{21,22} Because mitochondrial dysfunction is identified in most immune cells during SARS-CoV-2 infection,¹⁴ understanding how ADRS-induced hypoxia affect NKT function in COVID-19 is critically important.

The lung is the primary target organ of SARS-CoV-2, as the spike protein directly binds to ACE2 receptors expressed on the surface of lung epithelial cells (ECs).²³ As a result, severe COVID-19 is characterized by significant lung damage, resulting in decreased blood oxygen saturation (hypoxia), as well as increased serum lactate dehydrogenase (LDHA) level.^{11,24,25} Both downstream hypoxia signaling, and hyperlactatemia have been associated with pro-inflammatory cytokine syndrome and lymphocyte dysfunction.^{26,27} However, it is not completely understood how hypoxia in COVID-19 patients affects the metabolic phenotype of Tc through attenuating EC function in the lung of COVID-19 patients.

In this experiment using high dimensional flow cytometry and cutting-edge single cell metabolomics of PBMCs from hospitalized COVID-19 patients, it is demonstrated that metabolic disorders by hypoxia and anaerobic glycolysis induced dysfunctional CD8⁺Tc and NKTs during SARS-CoV-2 infection. An impaired, hypoxia triggered impaired memory cell differentiation in CD8⁺Tc of COVID-19 patients was shown. Finally, mitophagy was found to be an important regulator of immunometabolic function in CD8⁺Tc and ECs. Intriguingly, pharmacological inhibition of mitophagy via Mdivi-1 enhanced effector function as well as rescued memory differentiation function amongst CD8⁺Tc. Publicly available single cell sequencing datasets on the bronchoalveolar lavage fluid (BALF) and PBMCs from COVID-19 patients were reanalyzed to validate the metabolic reprogramming in CD8⁺Tc, NKTs, and ECs at transcriptomic level. Altogether, the current experiment provides key, insightful cellular and molecular mechanisms underlying a critical link between lung dysfunction, metabolic dysregulation, and impaired lymphocyte function during SARS-CoV-2 infection.

### Methodology

### Sample Acquisition

Blood from healthy donors was ordered from Research Blood Company. Blood samples from hospitalized COVID-19 patients or patients with COVID-19 like symptoms, however, testing negative for COVID-19 (COVID(−) patients), were collected at AdventHealth Hospital under protocols IRB #1668907 and #1590483 approved by AdventHealth IRB committee. Strict confidentiality was maintained for all patients according to HIPAA confidentiality requirements. COVID-19 positivity/negativity was confirmed by a PCR test at AdventHealth. Blood was used for human PBMC, plasma, and serum isolation.

### Patient Classification Criteria

The patient cohort consisted of hospitalized COVID(+) and COVID(−) patients requiring either emergency admission or acute IP care. The classifier “severe respiratory impairment” was used to describe patients recorded as having either “dependence on respirator (ventilator)”, “supplemental oxygen”, “acute respiratory failure”, “acute respiratory distress syndrome”, “hypoxia”, “hypoxemia”, “acute and chronic respiratory failure”, “chronic respiratory failure”. Additionally, the classifier “presence of dysfunctional lung symptoms” was used to describe patients who are recorded as having any of the above-described symptoms of “severe respiratory impairment” as well as “pneumonia”, chronic obstructive pulmonary disease”, “pneumonitis”, “pulmonary fibrosis”, “bronchiectasis”, “acute pulmonary edema”, “interstitial pulmonary disease”, “chronic pulmonary edema”, “dependence on other enabling machines and devices”. Patients considered as having metabolic syndrome/disorder were diagnosed as having “obesity”, “morbid obesity”, “type 2 diabetes mellitus”, “other unspecified diabetes mellitus”, “prediabetes”, “diabetes insipidus”, “type 1 diabetes mellitus”, “metabolic syndrome”. Patients considered as having prior transplantation and or/immunosuppression/dysfunction were diagnosed as having “organ transplant status/failure”, “bone marrow transplant”, “stem cell transplant”, “disorder involving the immune mechanism”, “immunodeficiency”, “history of immunosuppression therapy”, “human immunodeficiency virus (HIV) disease”.

### PBMC Isolation

The PBMC isolation methodology for Experiment 1 was used for Experiment 2.

### Antibody Staining and Flow Cytometry

PBMCs (0.5×10⁶ cells) were first stained with live/dead in PBS for 15 min, washed with Flow Cytometry Staining Buffer (FACS Buffer), and stained with surface markers in ice-cold FACS buffer at 4° C. for 30 min. PBMCs were then washed twice with FACS buffer and stained with secondary antibodies for 15 minutes. Samples were fixed and permeabilized using Fixation/Permeabilization buffer (20 min) at room temperature and washed with ice-cold FACS buffer. PBMCs were then stained with intracellular antibodies at 37° C. for 45 min in permeabilization buffer. Samples were washed once with permeabilization buffer before being resuspended in FACS buffer for flow cytometric analysis using Cytoflex system. Data was then analyzed by Flowjo™v10.

### Single-Cell Metabolism Uptake Assay

Mitochondrial membrane potential, protein translation, mitochondrial mass, and cytosolic ROS were assessed via uptake of TMRM, puromycin; mitotracker (MTR); and 2′,7′-dichlorofluorescein diacetate (DCFDA). Cells were incubated with fluorescent dye for 15 minutes at 37° C. before being washed with ice-cold FACS buffer followed by downstream flow cytometric staining in accordance with the aforementioned description.

### Single-Cell Metabolomics Assay (SCENITH)

PBMCs were incubated for 2 hr followed by a 20 min treatment with either 100 mM 2-deoxy-glucose (2-DG), 1 μM oligomycin (O), or a sequential combination of both drugs 37° C., 5% CO₂. Subsequently, puromycin (10 μg/mL) was added to the culture for additional 30 min. Afterwards, cells were washed with ice-cold PBS and subjected to downstream flow-cytometric staining. Cells were fixed, permeabilized, and stained intracellularly with the monoclonal anti-puromycin antibody for 45 min. Fluorescence was recorded in the FITC channel.

Values for “glucose dependence” and “FAO and AAO capacity” were calculated in accordance with the original SCENITH protocol.⁹⁰ Briefly, “glucose dependence” was calculated as 100*(CTL^{PuroMFI}−2−DG^{PuroMFI})/(CTL^{PuroMFI}−2−DG+O^{PuroMFI}). “FAO and AAO capacity” was calculated as 100−100*(CTL^{PuroMFI}−2−DG^{PuroMFI})/(CTL^{PuroMFI}−2−DG+O^{PuroMFI}). Additionally, for direct investigation of the dependence of overall cellular energy production on glycolysis, values for “glycolytic flux” as the percent decrease in puromycin^{high} cells after treatment with 2-DG compared to control as reported previously by Hong et al.⁹¹ were calculated. The formula used for this was 100*(% Puro^{+CTL}−% Puro^{+2-DG})/(% Puro^{+CTL}) Likewise, values for “mitochondrial flux” as percent decrease in puromycin^{high} cells after treatment with oligomycin compared to control were calculated. The formula used for this was 100*(% Puro+^{CTL}−Puro+^{Oligomycin})/(Puro+^{CTL}).

Similar to the guidance provided by Arguello et al.,⁹⁰ if the MFI value of puromycin is higher in the inhibitor treatment (either 2-DG or O) compared to the control, the glucose/mitochondrial dependence value was considered to be 0%. Additionally, if the percentage of puromycin positive cells is higher in the inhibitor treatment compared to the control, the glycolytic/mitochondrial flux was 0%. Likewise, if the MFI value of puromycin is lower in the singular inhibitor treatment (either 2-DG or O) compared to the combination treatment (2-DG+O), the dependence was 100%.

### CD3/CD28 Polyclonal Activation

96 well flat-bottom plates were coated with human for two hr at 37° C., 5% CO₂. PBMCs (5×10⁵) were activated with plated bound anti-CD3 (5 μg/mL) and soluble anti-CD28 (5 μg/mL) in complete culture media. After 48 hr, cells were stimulated with 50 ng/mM PMA and 1 μg/mL ionomycin followed by 3 hr incubation with GolgiStop to evaluate intracellular cytokine secretion. Cells were then subjected to flow cytometric staining as described above.

### SARS-CoV-2 Specific Peptide Activation

PBMCs (5×10⁵) were stimulated with 1 mg/mL of CD8a SARS-CoV-2 megapool peptide⁹² in the presence of absence of 20 μM Mdivi-1 at 37° C., 5% C02 for 96 hr.

### High-Dimensional Flow Cytometry Analysis

First, traditional bivariate gating using FlowJo™v10 was performed to identify major cell types. CD8+Tc and NKTs from each sample were identified, isolated, and exported to new fcs files. The flowCore package in R was used to read in concatenated CD8+Tc and NKT fcs files into the R environment.⁹³ Next, an arcsinh transformation was applied for data normalization. Data from all the samples were then merged into one catalyst object, upon which downstream analyses were performed.⁹⁴ PCA was then run on bulk sample-aggregated data and the top 3 principal components were plotted. FlowSOM clustering was performed on only cell surface markers used for phenotypic identification with the number of expected populations set at 30.95 Clusters were then annotated based upon canonical marker expression. Differential abundance of cell-type proportions and differential expression of MFI values were then conducted.

BALF scRNA SEQ Data Acquisition

The BALF scRNA SEQ data acquisition for Experiment 1 was used for Experiment 2.

PBMC scRNA SEQ Data Acquisition

Single cell RNA-seq data from a total of 8 COVID-19 patients and 4 healthy donors were reanalyzed from an existing study published by Lee et al.⁴⁹ For three out of the eight COVID-19 patients, two separate samples were collected at different time points for total 11 samples (6 severe COVID-19, 5 mild COVID-19, and 4 healthy) present in the cohort for analysis. The instant inventor defined COVID-19 disease severity as either moderate or mild using metrics from the National Early Warning Score methodology, where “respiratory rate, oxygen saturation, oxygen supplement, body temperature, systolic blood pressure, heart rate, and consciousness” were used as evaluating criteria.^{49,97} Prefiltered expression matrices with UMI counts were downloaded from the GEO Database with accession number GSE149698. Metadata was downloaded from the supplemental information provided in the original article.⁴⁹

Quality Control and Preprocessing of BALF and PBMC scRNA SEQ Data

The data quality control and preprocessing for Experiment 1 was used for Experiment 2.

### Tc Reintegration and Secondary Clustering in BALFs

The Tc reintegration for Experiment 1 was used for Experiment 2.

### Trajectory Inference and Pseudo-Temporal Ordering

The Trajectory Inference and Pseudo-temporal Ordering for Experiment 1 was used for Experiment 2.

### Metabolic Phenotype Based Clustering

The Metabolic Phenotype Based Clustering for Experiment 1 was used for Experiment 2.

### Network Analysis

The Network Analysis for Experiment 1 was used for Experiment 2.

### Downstream Analysis

The Downstream Analysis for Experiment 1 was used for Experiment 2.

### Statistical Analysis

Differential expression analysis of transcript abundance was assessed using Seurat's implementation of the nonparametric Wilcoxon rank-sum test. Genes were generally defined as statistically significant by Bonferroni adjusted p. value less than 0.05 and log-fold change greater than 0.25. For NKT, non-adjusted p. value was used to define differentially expressed genes due to very small sample size.

For comparison of cell-type proportion and MFI (either mean, median, or geometric median fluorescent intensity based upon the distribution) values, a two-tailed Student's t-test was performed to indicate statistical significance. Fisher's exact test was used for all comparisons of categorical variables in Table 5. Additionally, Pearson correlation coefficient was used to indicate strength of measured correlations. For correlation statistics involving categorical values, categorical or factorial variables were converted to binary “dummy” variables (either 0 or 1) for the purpose of statistical calculations.

Given the tremendous degree of lymphopenia in patient samples, in flow cytometric analysis, samples in which the total cell count of a cell population of interest was critically low to the point where it was not comparable to the other samples in the dataset were systematically excluded from downstream analysis. For data that was combined between different datasets, values were normalized by multiplication of a common factor to align the means between the different datasets.

### Results and Detailed Figure Description

### A) Results

### High-Dimensional Immunophenotyping Reveals a Distinct COVID-19 Immunophenotype in Both Circulation and the BALF.

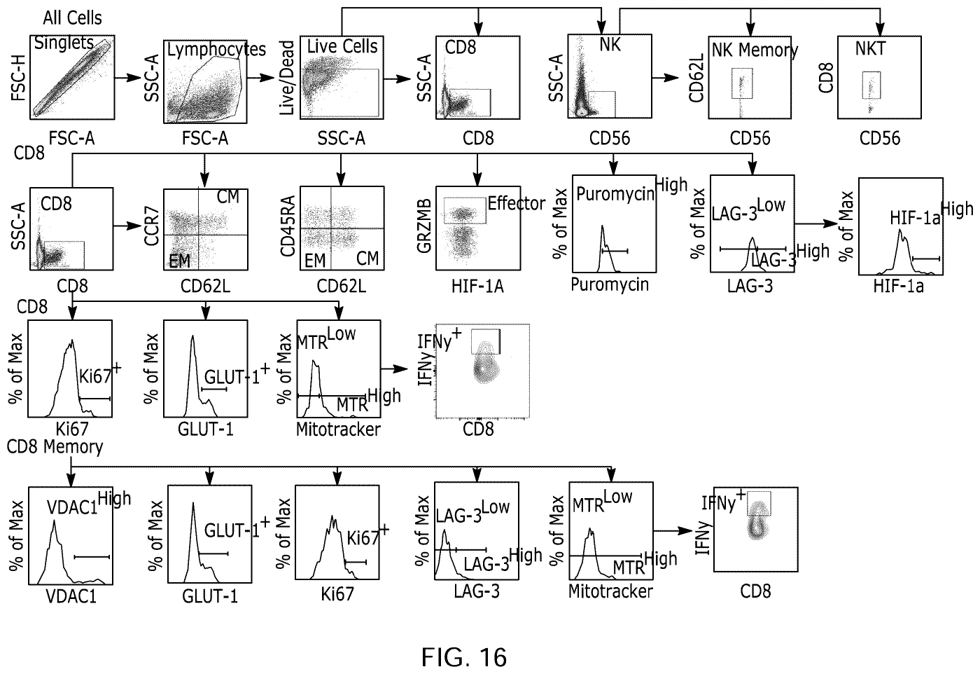

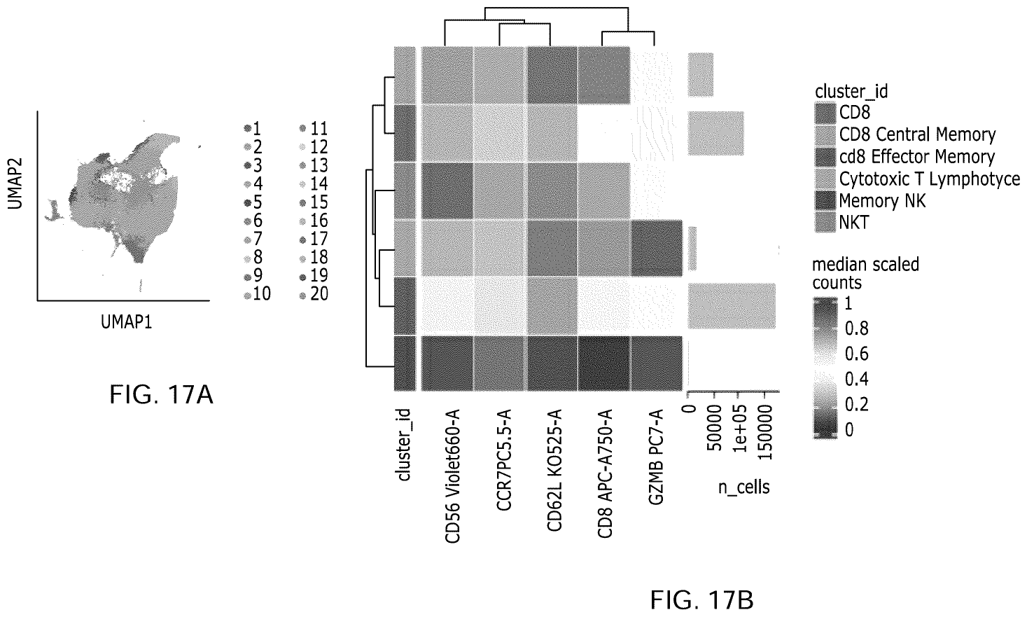

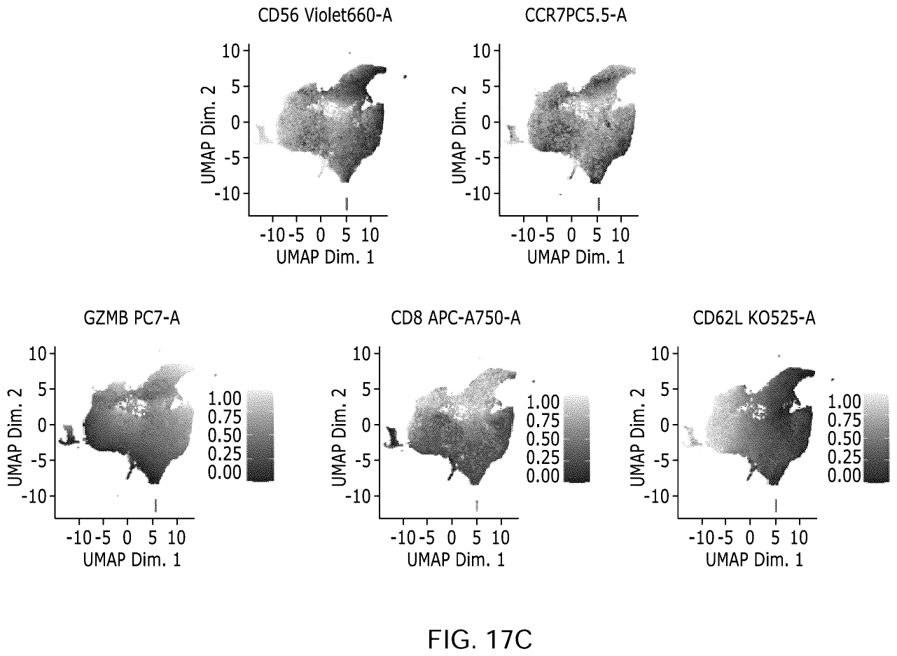

COVID-19 patients with severe disease have been found to suffer from significant immune dysregulation.^{28,29} High-dimensional immunophenotyping of peripheral blood mononuclear cells (PBMCs) from non-infected controls (healthy), hospitalized COVID-19 patients (COVID(+)), as well as from non-COVID (by PCR test) patients with COVID-like upper respiratory symptoms requiring intensive care, abbreviated as COVID(−) was first performed. (FIG. 1A, FIG. 16, Table 5). Analysis was performed on freshly isolated cells without cryogenic preservation to best reflect the metabolic/functional state of cells in the body. Comparative evaluation of patient samples by principal component analysis (PCA) revealed distinct clustering of healthy, COVID(−), and COVID(+) patients, suggesting an abnormal immunophenotype of PBMCs during SARS-COV-2 infection (FIG. 1B). CD8+Tc are the main cellular immune population that governs viral clearance.^{30,31} Corroborating prior reports, high-dimensional flow cytometry (FIGS. 17A-C) revealed significant lymphopenia of multiple CD8-derived subsets in PBMCs from hospitalized COVID(+) patients (FIGS. 1C, E). Bulk CD8⁺Tc were dramatically decreased in COVID(+) patients compared to both COVID(−) and healthy patients (FIG. 1E). The percentage of circulating effector CD8⁺Tc was increased in COVID(+) patients compared to healthy donors; however, significantly decreased compared to that of COVID(−) patients (FIG. 1E). Additionally, the percentage of circulating CD8⁺T_{M} was heavily reduced in severe COVID(+) patients as compared to healthy controls (FIGS. 1D, G). However, this consistent decrease was not noted amongst COVID(−) patients, indicating impaired memory differentiation specifically occurs in SARS-CoV-2 infection (FIGS. 1D, G).

Given that the lung is the primary target for COVID-19 attack,¹ whether CD8⁺Tc lymphopenia could also be detected in bronchoalveolar lavage fluid (BALF) cells was examined. A publicly available single cell RNA-sequencing (scRNA-seq) dataset of BALF samples from COVID(+) patients³² was reanalyzed. Tc were identified (FIGS. 9A-B), subsetted, and a second round of unsupervised clustering and UMAP dimensionality reduction was performed to identify and delineate distinct, clear Tc subsets (FIGS. 1H, I and FIG. 10). Similarly, effector CD8⁺Tc were significantly increased in the BALF of COVID(+) patients compared to healthy controls (FIGS. 11, K). Noticeably, patients with mild as compared to severe symptoms had dramatically higher abundance of effector CD8⁺Tc in the BALF (FIGS. 11, K). Finally, a significant attenuation in the percentage of CD8⁺T_{M} in COVID-19(+) BALFs, which was more apparent in severe patients (FIGS. 11, L), corroborating the impaired memory cell abundance found in PBMCs (FIGS. 1D, G) was observed.

TABLE 5

| | Hospitalized COVID(−) | | COVID-19 | | |
|---|---|---|---|---|---|
| Clinical Characteristics | (n = 36) | | (n = 52) | | P value |
| Average Age (y) | 67.74 | | 64.04 | | 0.1792 |
| Male Gender | 18/36 | (50%) | 28/52 | (53.85%) | 0.8289 |
| Race (White) | 22/36 | (61.11%) | 26/52 | (50.00%) | 0.3849 |
| Race (Black or African American) | 7/36 | (19.44%) | 13/52 | (25.00%) | 0.6118 |
| Prior transplantation and/or | 5/36 | (13.88%) | 8/52 | (15.38%) | 1 |
| immunosuppression/dysfunction | | | | | |
| Prior type 2 DM/metabolic disorder | 20/36 | (55.55%) | 37/52 | (71.15%) | 0.1471 |
| Prior hypertension | 24/36 | (66.66%) | 29/52 | (55.77%) | 0.3776 |
| Prior cardiac arrhythmia | 0/36 | (0%) | 0/52 | (0%) | 1 |
| Hospitalization | 36/36 | (100%) | 52/52 | (100%) | 1 |
| Supplemental oxygen or mechanical | 1/36 | (2.77%) | 7/52 | (13.46%) | 0.134 |
| ventilation | | | | | |
| Severe respiratory impairment | 12/36 | (33.33%) | 28/52 | (53.85%) | 0.0814 |
| Presence of dysfunctional lung | 21/36 | (58.33%) | 40/52 | (76.92%) | 0.0989 |
| symptoms | | | | | |
| Admission to emergency room | 35/36 | (97.22%) | 50/52 | (96.15%) | 1 |
| Average time of sample collection | 12.14 | | 14.41 | | 0.403 |
| (# days after hospitalization) | | | | | |
| Laboratory Parameters | | | | | |
| Elevated lactate dehydrogenase | 6/36 | (16.67%) | 17/52 | (32.69%) | 0.138 |
| Elevated C-reactive protein | 14/36 | (38.89%) | 25/52 | (48.08%) | 0.5131 |

### CD8⁺Tc Metabolically Reprogram Towards Glycolytic Dependence and Exhibit Impaired Mitochondrial Function During SARS-CoV-2 Infection.

Metabolic dysregulation is well recognized in COVID-19 pathogenicity and has been linked to a dysfunctional immune response.³³ The function and immunological fate of CD8⁺Tc specifically has been shown to be heavily dependent on metabolism during viral infection.⁸ The immunometabolic profiles of CD8⁺Tcs on freshly isolated patient-derived PBMCs was assessed. Because hypoxia and excessive glycolysis are evident in COVID(+) patients with severe disease,^{25,34,35} the degree of glucose uptake in CD8⁺Tc during SARS-COV-2 infection was first examined. Glucose transporter 1 (glut-1) is a receptor that facilitates glucose uptake by Tc during viral infection.³⁶ Indeed, expression of glut-1 was augmented in CD8⁺Tc of COVID(+) patients (FIG. 2E). To assess the cellular dependence of COVID-19(+) CD8⁺Tc on glycolysis, SCENITH single-cell metabolomics assay was leveraged. COVID(+)CD8⁺Tc had increased glycolytic flux compared to healthy and COVID(−) patients (FIG. 2F). Accelerated glut1 expression and glycolytic flux in CD8⁺Tc from COVID19(+) compared to those from COVID(−) patients (FIGS. 2E-F) suggested dysfunctional CD8⁺Tc glucose metabolism as a hallmark of SARS-CoV-2 infection. Under hypoxic condition, excessive dependence on glycolytic flux has been linked to mitochondrial dysfunction.³⁷ As expected, decreased mitochondrial membrane potential, indicative of impaired mitochondrial function was selectively noted in COVID(+) CD8⁺Tc (FIG. 2G). Consistently, SCENITH metabolomics analysis revealed a strong reduction in the mitochondrial flux of COVID(+) CD8⁺Tc compared to those from both healthy and COVID(−) patients (FIG. 2H). Together, these results clearly indicated a functional metabolic switch from mitochondrial respiration to glucose-dependent metabolism in CD8⁺Tc during SARS-CoV-2 infection. Prolonged anaerobic and mitochondria-independent glycolysis was reported to impair reductive NADPH activity, resulting in the propagation of oxidative stress.^{38,39} As predicted, an accumulation of cytoplasmic reactive oxygen species (ROS) was detected in CD8⁺Tc from COVID-19 (+) patients (FIG. 2I). Mitochondrial FAO is an important mechanism for cells to prevent excessive oxidative stress production⁴⁰ and to generate acetyl-CoA (Ac-CoA) required maintaining stemness.⁴¹ Indeed, decreased expression of carnitine palmitoyltransferase 1A (cpt1a), an enzyme catalyzed the transfer of long chain FA through mitochondrial membrane for subsequent oxidation,⁴² was also found indicating attenuated FAO in CD8⁺Tc from COVID-19 patients (FIG. 2B). Additionally, enhanced expression of LC3 (FIG. 2K) indicated prevalence of CD8⁺Tc autophagy in COVID-19. Along with mitochondrial impairment, these results demonstrated upregulation of mitophagy in CD8⁺Tc during SARS-CoV-2 infection.

### Significant Metabolically Linked Exhaustion in CD8*Tc During Severe SARS-CoV-2 Infection.

Given that metabolism is a critical regulator of immune cell function,⁴³ the functional characteristics of CD8⁺Tc during COVID-19 were examined. Augmented expression of surface glycoprotein lymphocyte activation gene-3 (lag-3) is indicative of increased cellular exhaustion of CD8⁺Tc in COVID(+) patients (FIG. 2L). It was further found that cellular protein synthesis, which is required for production of effector molecules and cytokines in activated Tc,⁴⁴ was reduced in COVID(+) as compared to COVID(−) CD8⁺Tc (FIG. 2M). In support of this observation, remarkable decrease in ki67 expression was detected in CD8⁺Tc from COVID(+) patients (FIG. 2N) validating an impairment in CD8⁺Tc proliferation during SARS-CoV-2 infection. Increased Hif-1α was detected during severe SARS-CoV-2 infection because of reduced oxygen saturation and hypoxia.^{45,46} Notably, there was a significantly increased expression of Hif-1α in lag-3^{high}CD8⁺Tc from COVID(+) patients (FIG. 20) suggesting that a hypoxia-mediated metabolic switch may implicate in CD8⁺Tc dysfunction. Along this line, COVID-19(+) CD8⁺Tc with lower mitochondrial mass exhibited impaired IFNy secretion capacity (FIG. 2P) were found.

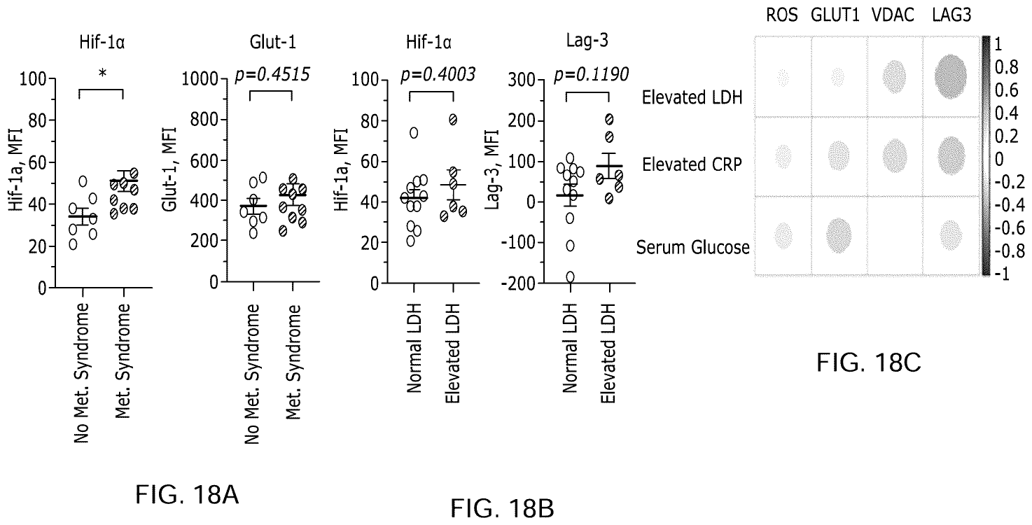

To investigate whether immunometabolic dysregulation in CD8+Tc can be a potential mechanism underlining increased COVID-19 severity within patients with metabolic disorders, whether COVID(+) patients in the cohort, who had metabolic syndrome possessed differential immunometabolic profile, was evaluated. A significant increase in the expression of Hif-1α on CD8⁺Tc amongst COVID(+) patients with metabolic syndrome was observed, accompanied by a mild increase in glut-1 expression, suggesting that patients with prior metabolic comorbidities may have an increased hypoxia-driven anaerobic glycolysis (FIG. 18A). In patients who had elevated level of serum lactate dehydrogenase (LDH) (key glycolysis rate-limiting enzyme), a moderate increase in the expression of Hif-1α and lag-3 (FIG. 18B) was found, further highlighting the potential connection between altered patient metabolism and dysregulated immune function during SARS-CoV-2 infection. Accordingly, positive correlations between serum glucose level and lag-3 or glut1 expression in CD8⁺Tc and between LDH and CD8⁺Tc expression of lag-3 and VDAC1 were identified in COVID-19 patients (FIG. 18C).

### Hypoxia is the Driven Factor for Metabolic Reprogramming in COVID-19(+) CD8⁺Tc.

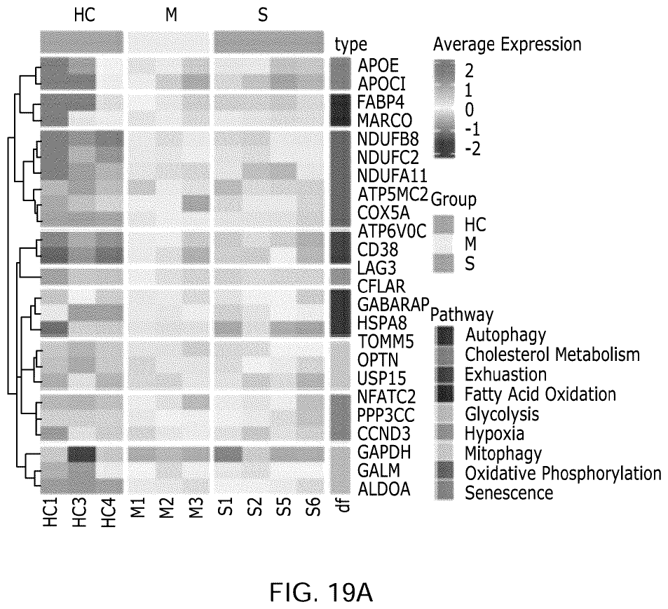

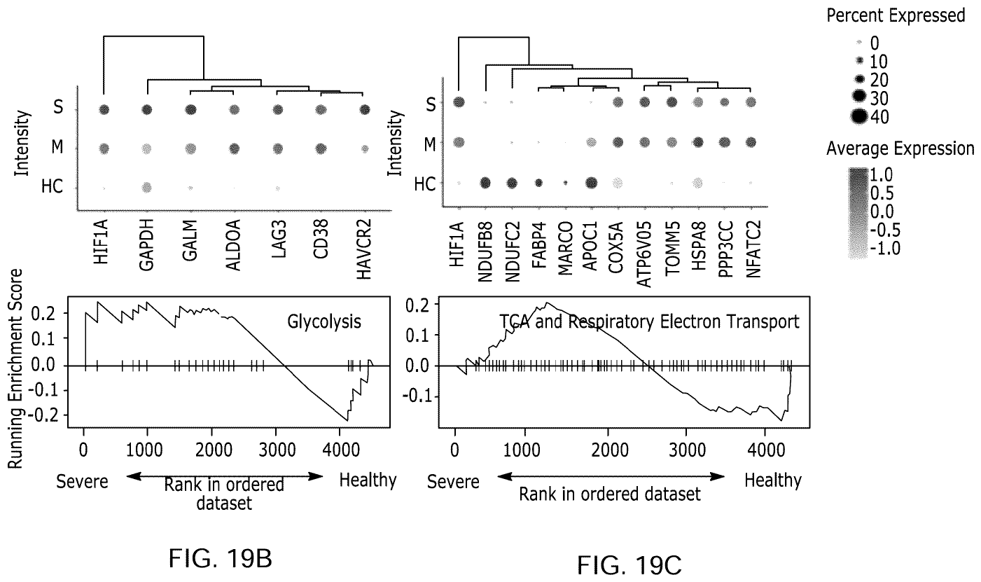

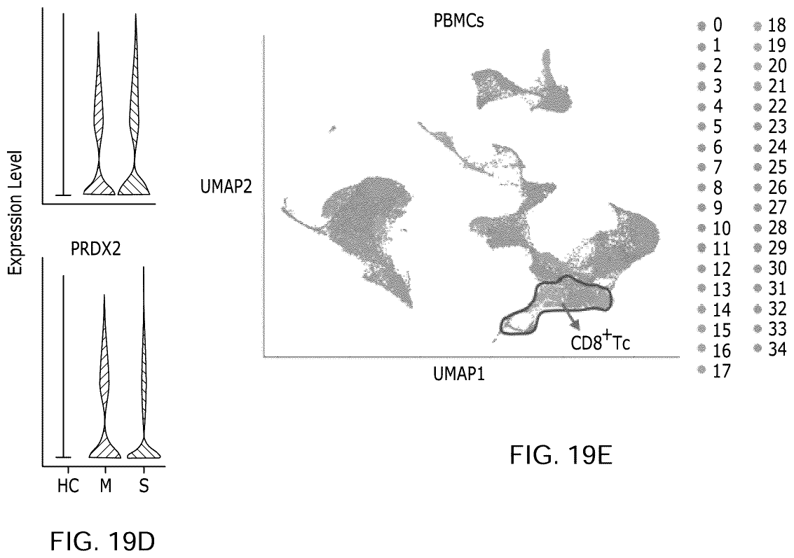

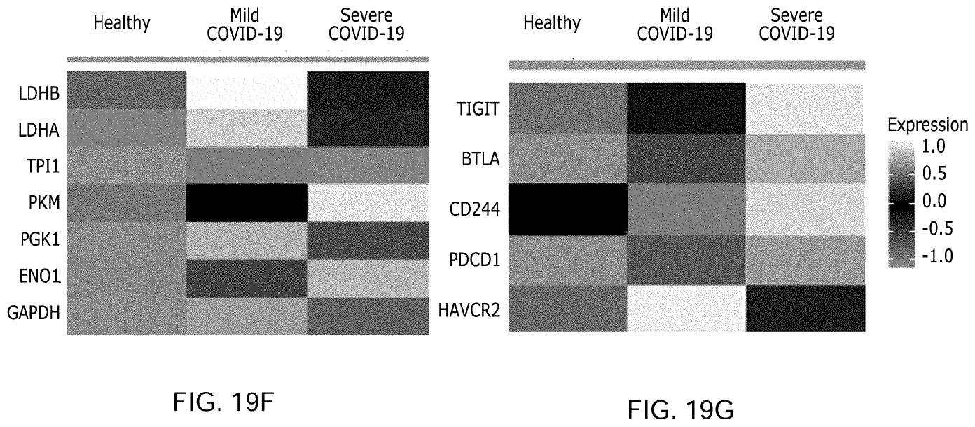

To better understand the transcriptional changes underlying the relationship between metabolic dysfunction and impaired immune cell function in the primary site of viral attack, the metabolic landscape of BALF effector CD8+Tc from COVID(+) patients was profiled. Differential expression analysis revealed increased expression of genes encoding anaerobic glycolysis (GAPDH, GALM, andALDOA) in effector CD8+Tc from moderate and severe COVID(+) patients (FIGS. 19A-B). Key metabolic pathways including hypoxia, anaerobic glycolysis, mitophagy, autophagy, cell exhaustion, and senescence were upregulated, while pathways relying on mitochondrial metabolism including FAO, cholesterol metabolism, and oxidative phosphorylation (OXPHOS) were attenuated in effector CD8+Tc from COVID(+) patients (FIGS. 19A, C). Hierarchical clustering suggested a tight association between Hif-1α and anaerobic glycolysis (FIG. 19B), indicating that oxygen-deprived condition in the BALF environment is linked to anaerobic glucose metabolism. GSEA analysis showed that effector CD8⁺Tc were comparatively less dependent on mitochondrial metabolism during SARS-CoV-2 infection (FIG. 19C). Reduction of NAD⁺ to NADH conversion is required to preserve cellular redox homeostasis and sustain glycolytic flux.³⁹ Decreased expression of transcripts encoding NADH oxidoreductases (NDUFB8, NDUFC2, and NDUFA11) in effector CD8⁺Tc from COVID(+) patients (FIGS. 19A, C) was observed. There was also downregulation of lipid metabolism-associated genes (FABP4, APOC1, APOE, MARCO) in COVID-19 effector CD8⁺Tcs (FIGS. 19A, C). Increased oxidative stress was also evident by overexpression of NFE2L2 and PRDX2 (FIG. 19D). Decreased NADH oxidation and a concomitant increased NAD⁺ are associated with impaired cytokine secretion, cell proliferation, and exhaustion.^{47,48} Indeed, the expression of CD38, an NAD⁺ hydrolase linked to Tc exhaustion,⁴⁸ was increased in COVID-19 effector CD8⁺Tc (FIGS. 19A-B). These results prove that hypoxia-induced CD38 expression is associated with metabolic reprograming and cellular exhaustion in the lung of COVID(+) patients. This conclusion is supported by higher levels of exhaustion markers LAG3 and TIGIT in effector CD8⁺Tc from severe COVID(+) patients (FIGS. 19A-B). In order to validate the observed transcriptomic changes in the BALF, an existing single cell transcriptomic profiling dataset from COVID-19 patient PBMCs⁴⁹ was reanalyzed. In bulk CD8+Tc identified by unsupervised clustering, a strong increase in the expression of key glycolytic genes in COVID(+) patients (FIGS. 19E-F) was also observed. Additionally, the expression of transcripts regulating cellular exhaustion (TIGIT, BTL4, PDCD1, and HAVCR2) was found to be similarly upregulated in severe COVID-19 (FIG. 19G). Altogether, these results consistently suggest that hypoxia arising from COVID-19-pulmonary dysfunction augments glycolytic flux, impairs FAO and oxidative stress, leading to mitochondrial dysfunction and immunological exhaustion.

Impaired Memory Cell Differentiation in CD8⁺Tc from Patients with Severe COVID-19.

Memory CD8⁺Tc (CD8⁺T_{M}) can provide protective immunity against secondary viral infection.^{50,51} To understand the dynamics of memory differentiation in CD8⁺Tc during SARS-CoV-2 infection, trajectory inference and pseudo-temporal modeling analysis for BALF CD8⁺Tc (FIG. 4A) was performed. Differential analysis demonstrated a strong reduction of pseudotime for CD8⁺T_{M} in severe compared to moderate COVID-19 and healthy control patients (FIGS. 4A-C). In contrast to reduced CD8⁺T_{M}, the enrichment of proliferating and effector CD8⁺Tc was identified in moderate and severe compared to healthy BALF cells (FIG. 4C). Notably, these findings are also corroborated in patient PBMCs, where the proportion of effector CD8⁺Tc are increased in severe and moderate patients compared to healthy donors, however the proportion of memory cells are severely decreased in comparison to both COVID(−) and healthy patients (FIGS. 1E-G). These findings suggest that CD8+Tc are stalled along their memory differentiation trajectory and are unable to reach the terminal state during severe COVID-19 infection. During viral infection, circulating memory cells migrate to the infected tissue and differentiate into tissue-resident memory (TRM) cells to provide the first response against pathogen reencounter.⁵² Reduced expression of tissue residence-indicating genes ITGA1 and ZNF683 selectively in severe COVID(+) patients (FIG. 4D) was observed. This result suggests that impaired differentiation of T_{RM} was also evident in the lung of severe COVID-19 patients, which may be implicated with attenuated capacity for viral clearance of CD8⁺Tc during SARS-COV-2 infection. However, no dysregulation of memory differentiation was found in CD4⁺Tc of COVID(+) patients (FIGS. 13A-B), suggesting this phenomenon selectively occurred in CD8⁺Tc.

As memory differentiation is strictly regulated by metabolism,^{53,54} the potential relationship between infection-induced metabolic dysregulation and memory cell differentiation in CD8⁺Tc was investigated. Increased glucose uptake, indicated by augmented glut-1 expression, as well as elevated dependence on glucose metabolism, demonstrated through single-cell SCENITH analysis, were observed in CD8⁺T_{M} from COVID(+) patients (FIGS. 3G-H). Additionally, a decreased capacity of mitochondria to oxidize amino acid (AA) and FA was found in COVID-19⁺ CD8⁺T_{M} (FIG. 3H). During cellular stress, excessive ROS production may be associated with increased electron leakage at the sites of complex I (NADH-ubiquinone oxidoreductase) and complex III (ubiquinone-cytochrome c oxidoreductase) in the electron transport chain (ETC), resulting in dysfunctional mitochondrial activity.^{55, 56} The results demonstrated increased ROS was solely observed in CD8⁺T_{M} from COVID(−) but not from COVID(+) patients (FIG. 3I) suggesting that ROS accumulation in CD8⁺T_{M} is nonspecific for COVID-19. However, a selective increase in COVID(+) patients of voltage-dependent anion channel (VDAC), involved in cellular redox and mitochondria-mediated apoptotic signaling, was identified (FIG. 3J), suggesting impaired mitochondrial integrity in COVID-19(+)CD8⁺T_{M}. Accordingly, decreased mitochondrial membrane potential was identified, further validating hampered mitochondrial function in CD8⁺T_{M} during SARS-CoV-2 infection (FIG. 3K). Evaluating the impact of dysregulated metabolism on cellular function, it was found CD8+TM exhibited increased cellular exhaustion during SARS-CoV-2 infection, as evidenced by upregulated expression of lag-3 (FIG. 3L). Consistently, CD8⁺T_{M} from COVID(+) patients demonstrated impaired cytolytic function, illustrated by decreased proportion of granzyme B (GrzmB)⁺ cells (FIG. 3M). Mitochondrial mass is a key regulator of cytokine-secreting capacity of CD8⁺T_{M}⁵⁷. An elevated IFNy secretion in COVID(+) CD8+TM with preserved mitochondrial mass (FIG. 3N) was observed. SCENITH analysis further confirmed that lag-3^{high}CD8⁺T_{M} were more metabolically dependent on glucose metabolism in COVID(+) patients (FIG. 30). Meanwhile, oxidation of FAO and AA in mitochondria was significantly reduced in lag-3^{high}CD8⁺T_{M} (FIG. 30), validating that a prolonged shift towards glucose metabolism is a key driver of mitochondrial impairment in exhausted CD8⁺T_{M} during SARS-CoV-2 infection.

Metabolic Dysregulation Triggers CD8⁺T_{M} Exhaustion in COVID-19.

GSEA analysis revealed that CD8+TM from severe or moderate COVID(+) patients were highly dependent on glycolysis for their bioenergetics demands (FIGS. 3A-B). Clustering and shared upregulation of glycolytic enzyme encoding genes GALM, GAPDH, GPI, and ALDOA together with Hif-1α, and transcripts regulating exhaustion (TIGIT and LAG3) (FIG. 3B) suggested that hypoxia/anaerobic axis is associated with impaired CD8⁺T_{M} function in COVID-19 BALF. FAO and OXPHOS promote the development of CD8⁺T_{M} after antigen exposure.⁵⁸ Indeed, genes-encoding regulators of lipid uptake (APOE, and APOC1) and FAO (OLR, MARCO, FABP4) were downregulated in CD8⁺T_{M} from severe COVID(+) patients (FIGS. 3A, C). Consistently, decreased expression of OXPHOS-coding genes was found in severe COVID (+) CD8⁺T_{M} (FIGS. 3A, C). A negative correlation of FAO-coding transcripts and Hif-1α expression (FIG. 3C) indicated that impaired mitochondrial metabolism is a result of hypoxia during SARS-CoV-2 infection. Strikingly, Pearson analysis revealed a negative correlation (R=−0.73, p=0.011) between module scores for glycolysis and FAO (FIG. 3D), which further emphasizes a potential association between prolonged anaerobic glycolysis and reduced mitochondrial fitness. In support of this, a strong positive correlation between module scores for glycolysis and exhaustion (R=0.85, p=0.00026) (FIG. 3D) validates that excessive glycolytic dependence is tightly associated with CD8+TM exhaustion. Genes involved in cellular senescence and mitophagy were upregulated in severe COVID-19 CD8+TM (FIG. 3A), implying that CD8⁺T_{M} metabolically switch to these pathways in response to impaired mitochondrial metabolism. Likewise, glutaminolysis was used as an alternative pathway, evidenced by upregulation of glutamate oxidation-regulating genes (GLUD1, DGLUCY) in COVID-19(+) CD8⁺T_{M} (FIG. 3A).

PCA analysis performed on 30 differentially expressed metabolic genes (Table 7) showed distinct clustering of CD8⁺T_{M} across different groups, further highlighting metabolic disorder during SARS-CoV-2 infection (FIG. 3E). Pearson correlation analysis showed a positive correlation between expression of genes-regulating glycolysis, mitophagy, senescence, and glutaminolysis (FIG. 3F). These genes were inversely correlated with transcripts regulating FAO and NADH oxidation (FIG. 3F). Increased CD38 expression in severe COVID-19 CD8⁺T_{M} was closely clustered with exhaustion-coding genes (LAG-3, TIGIT) (FIG. 3B) suggesting CD38 expression is associated with metabolic reprograming, memory impairment, and cellular exhaustion of CD8⁺T_{M} in the lung of COVID(+) patients. Together, these data demonstrated that the hypoxia/anaerobic glycolysis axis mediates CD8⁺T_{M} cellular dysfunction and exhaustion at transcriptomic level in COVID-19.

TABLE 6

| Cell type | Panel for metabolic phenotype-based clustering |
|---|---|
| CTL | APOE, APOC1, FABP4, MARCO, NFUFB8, NDUFC2, NDUFA11, ATP5MC2, |
| | COX5A, ATP6V0C, CD38, LAG3, HIF1A, CFLAR, GABARAP, HSPA8, |
| | TOMM5, OPTN, USP15, NFATC2, PPP3CC, CCND3, GAPDH, GALM, ALDOA |
| CD8 | APOE, APOC, OLR1, MARCO, FABP4, GABARAP, CFLAR, HSPA8, CCND3, |
| Memory | PPP3CC, NFATC2, DGLUCY, GLUD1, CD38, TIGIT, LAG3, GAPDH, GALM, |
| | GPI, ALDOA, HIF1A, TOMM5, USP15, OPTN, NDUFB8, OSP15, OPTN, |
| | NDUFB8, ATP6V1E1, NDUFA1, ATP5MC2, COX5A, ATP6V0C |
| Epithelial | IGFBP3, CDKN1A, GADD45B, ADH1C, ALDOA, GAPDH, PCK2, ENO1, |
| | ALDH1A3, GABARAP, SQSTM1, HIF1A, CTSB, CTSL, NDUFC2, NDUFB8, |
| | ATp5PO, UBB, MDH1, SDHC, ACADM, ACI2, SLC27A2, FABP6, APOC1, |
| | VDAC3 |
| NKT | FABP4, OLR1, SCP2, ACADM, HIF1A, GAPDH, LDHA, TPI1, PGAM1, ALDOA, |
| | LAG3, CD44, CD38, NDUFA11, NDUFA13, NDUFB8, ATp5MC2, NDUFA12, |
| | ATP6V0C, COX5A, CTSL, CFLAR, ITPR1, UBB, KRAS, CSNK2A1, SLC25A5, |
| | GAFF45B, JUN, PRF1, IFNG, TNFSF10, HCST, LRC2, KLRC1, APOE, APOC1 |

TABLE 7

| | | | HLA Class 2 | Type 1 | Nf-kB |
|---|---|---|---|---|---|
| | Glycolysis | FAO Module | Signaling | Interferon | Module |
| | Module Score | Score | Module Score | Module Score | Score |
| CD8 | GAPDH, | OLR, | | | |
| Memory | GALM, GPI, | MARCO, | | | |
| | ALDOA | FABP4 | | | |
| Epithelial | ADH1C, | ACADM, | HLA-DRA, | IFITM1, | SQSTM1, |
| | ALDOA, | ECI2, | HLA-DPA1, | IFITM2, IFIT1, | GADD45B, |
| | GAPDH, | SLC27A2, | HLA-DMA, | MX1, IFITM2 | NFKBIA, |
| | PCK2, ENO1, | FABP6 | DYNLL1 | | RELB |
| | ALDH1A3 | | | | |

### Pharmacological Inhibition of Mitophagy Enhances Cellular Function of SARS-CoV-2 Specific-CD8⁺Tc.

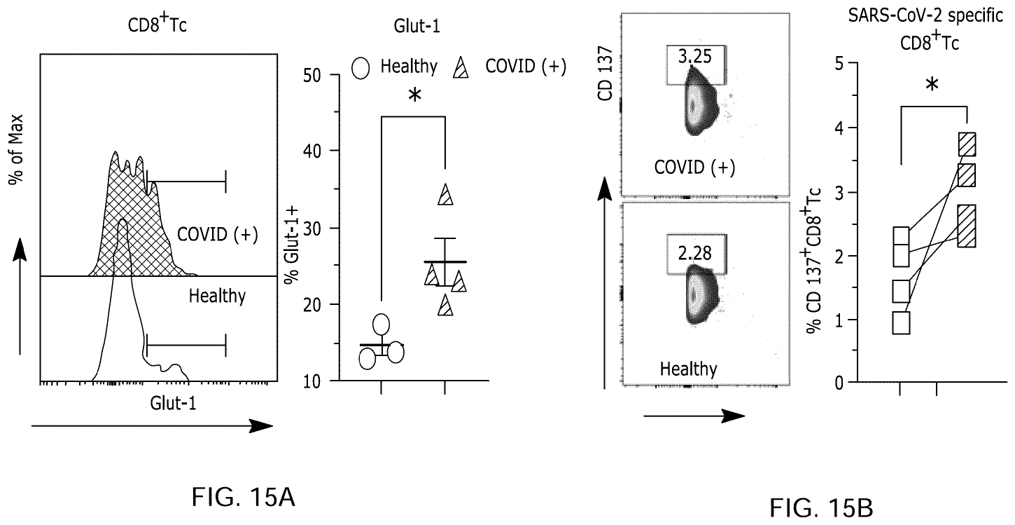

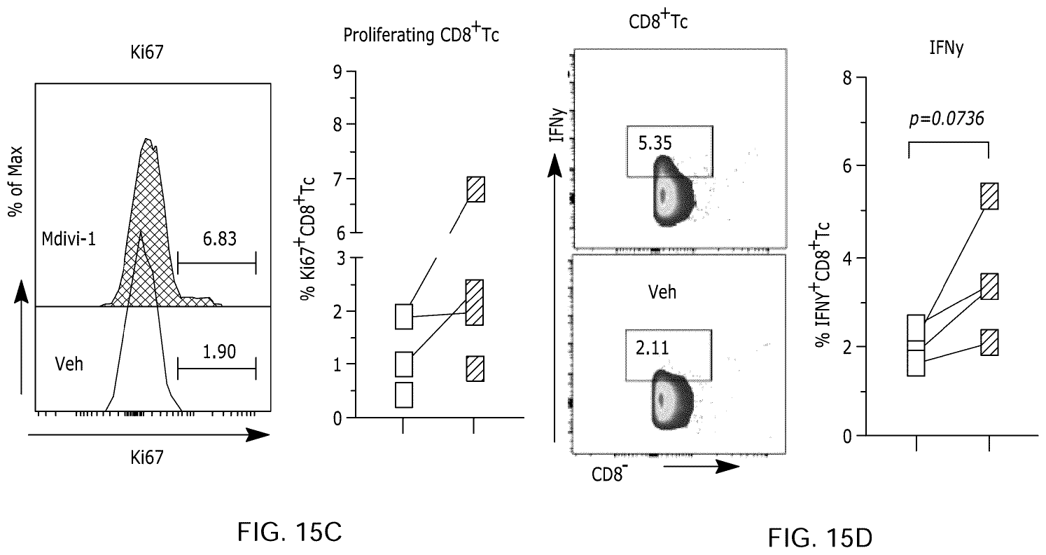

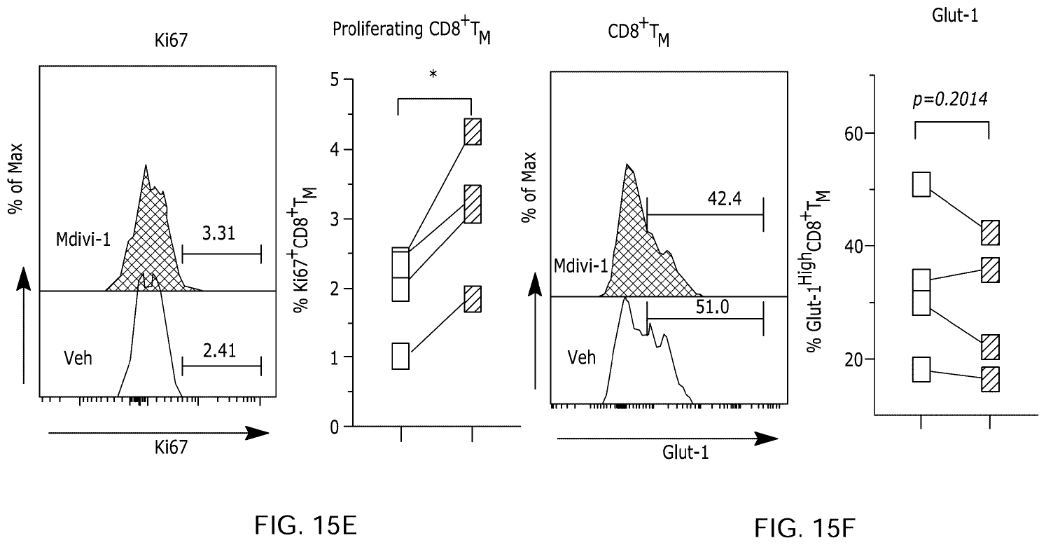

SARS-CoV-2 antigen-specific CD8+Tc in acute patients governs the intensity of adaptive immune response against SARS-CoV-2 infection.⁵⁹ COVID(+) PBMCs with a CD8a SARS-CoV-2 spike peptide megapool⁶⁰ was activated. It was found that SARS-CoV-2 CD8a peptide induced glut-1 expression in CD8⁺Tc from severe COVID(+) patients, suggesting that glucose metabolism is associated with SARS-CoV-2 specific CD8+Tc during infection (FIG. 15A). Given increased mitophagy found in CD8⁺Tc from PBMCs (FIG. 2K) and BALFs (FIGS. 19A, C) of COVID-19 patients, whether treatment with mitophagy inhibitor Mdivi-1 could enhance the cellular function of CD8⁺Tc and CD8⁺T_{M} after activation with SARS-CoV-2 peptide was investigated. Indeed, it was found that Mdivi-1 improved the generation of SARS-CoV-2-specific CD137⁺ cells (FIG. 15B), induced proliferation (FIG. 15C) and IFNy-secreting capacity CD8⁺Tc (FIG. 15D). Interestingly, Mdivi-1 treatment augmented CD8⁺T_{M} proliferation (FIG. 15E). Glut-1 expression was reduced in CD8⁺T_{M} under Mdivi-1 treatment (FIG. 16F), suggesting that inhibition of mitophagy may reverse dysregulated glucose metabolism and normalize memory differentiation in CD8⁺T_{c}. Collectively, these results demonstrate that pharmacological inhibition of mitophagy may restore metabolic disorder to improve the efficacy of the CD8⁺Tc response in COVID-19.

### Aberrant Metabolism Causes NKT Dysfunction in COVID-19.

NKTs, expressing CD56 and CD8, are intermediate between the CD8 and NK cell lineages.¹⁹ NKTs play critical roles in preventing pneumonia during chronic pulmonary disease.⁶¹ SARS-CoV-2 infection impairs NKTs effector functions and hinders the effective clearance of virally infected cells.¹⁹ Circulating NKT frequency has been implicated as a powerful prognostic biomarker of COVID-19 severity.¹⁸ Consistently, in the current cohort, NKT lymphopenia was observed in both PBMCs and BALF from COVID(+) patients (FIGS. 7F-G). Examination of the metabolic profile of NKTs from patient PBMCs revealed a significant increase in the expression of glut-1 in NKTs from COVID(+) patients compared to those from both healthy and COVID(−) counterpart (FIG. 7H), suggesting that augmented glucose utilization by NKTs is selectively occurs during SARS-CoV-2 infection. SCENITH analysis validated a trending increase in the glycolytic flux of COVID(+) NKTs (FIG. 7I). Consistently, enhanced ROS accumulation accompanied by a concurrent upregulation of VDAC, indicative of impaired mitochondrial fitness was detected in COVID(+) NKTs (FIGS. 7J-K). Accordingly, mitochondrial membrane potential was found reduced in NKT from COVID-19 patients (FIG. 7L). NKTs exhibit protective activity against viral infection via the secretion of cytolytic molecules such as grzmB.⁶² Consequently, decreased expression of grzmB in COVID(+) NKTs indicated reduced effector function of this cell subset during SARS-CoV-2 infection (FIG. 7M). To further probe the perturbed metabolism in NKTs from COVID-19 patients, gene expression profiles of BALF NKTs were characterized. Co-expression of CD8A andKLRD1 was used to define NKT lineage (Table 8). A transcriptional program associated with hypoxia-induced metabolic reprogramming was seen in NKTs from severe COVID-19 patients (FIG. 7A). Consistent to increased glut-1 expression (FIG. 7H), upregulation of anaerobic glycolytic metabolism involving genes (LDHA, TPi, PGAM1, ALDOA) were identified in severe COVID-19 NKTs (FIG. 7F). GSEA analysis validated a strong, concomitant increase in glycolysis genes (FIG. 7G). Importantly, a significant increase in the expression of genes-regulating TCA and respiratory electron transport was also seen (FIG. 7G). Under normal conditions, activated NKTs will expectedly use pyruvate dehydrogenase (PDHA1/2) to supply acetyl-CoA for mitochondrial TCA cycle metabolism.⁶³ However, under reduced oxygen saturation in the lung of COVID-19 patients, downregulation of genes encoding lipid uptake, FAO, and NADH oxidation (NDUFB8, NDUFA11, NDUFA13) was found in severe COVID(+) NKTs (FIGS. 7G-H) suggesting mitochondrial dysfunction in NKTs during SARS-CoV-2 infection. This observation may illustrate that glycolysis-derived pyruvate is converted into lactate rather than oxidized in TCA cycle leading to a lack of materials for sufficient OXPHOS to support NKT effector function during SARS-CoV-2 infection. Alternatively, increased levels of GADD45B and SLC25A5 transcripts (FIG. 7F) suggest metabolic adaptation via enhancement of mitophagic activity in NKTs during SARS-CoV-2 infection.

TABLE 8

| | Surface Markers Used |
|---|---|
| PBMC Cell Population | for Identification |
| Cytotoxic T Lymphocytes | CD8⁺, GRZMB⁺ |
| CD8 Central Memory Cells | CD8⁺, CD62L⁺, and CCR7⁺, or |
| | CD8⁺, CD45RA⁻, CD62L⁺ |
| CD8 Effector Memory Cells | CD8, CD62L⁻, CCR7⁻, or CD8⁺, |
| | CD45RA⁻, CD62L⁻ |
| NK Cells | CD56 |
| NKT Cells | CD56, CD8 |
| CD62L⁺ NK Cells (Memory NK) | CD56, CD62L |

### Metabolic Dysregulation Impairs Immune Surveillance and Increases Pro-Inflammatory Response in Lung Epithelial Cells During SARS-CoV-2 Infection.

Epithelial cells (ECs) secrete cytokines and help mediate antigen presentation to modulate immune cells function during viral infection.⁶⁴ Differential expression analysis revealed overexpression of key immune signaling pathways in COVID-19 ECs (FIG. 6A). Induction of a pro-inflammatory cascade including type 1 IFN, toll-like receptor, NF-kB, and chemokine signaling was observed in COVID-19 ECs (FIG. 6B). Glucose metabolism mediates type I IFN secretion through enhancing NF-kB expression⁶⁵ and epigenetic acetylation⁶⁶. Indeed, a positive correlation between module scores for glycolysis and type 1 IFN signaling (FIG. 6C) as well as for glycolysis and NF-kB signaling (FIG. 6D) was found. Chronic presentation of viral antigens to CD8⁺Tc by ECs may cause cellular dysfunction.⁶⁷ It was observed that genes encoding HLA class 1 (HLA-E, PSMA-6, TAP1, IF130) were enriched in COVID-19 ECs (FIG. 6A). GSEA analysis further confirmed the upregulation of HLA class 1 antigen presentation in bulk ECs (FIG. 6E). In contrast, downregulation of genes encoding HLA class 2 (HLA-DRA, HLA-DPA1, HLA-DMA, DYNLL1) was found in COVID-19 ECs (FIG. 6A) which was further confirmed by GSEA analysis (FIG. 6E). Glycolysis was reported to repress functional response of antigen presenting cells during infection.⁶⁸ A negative correlation of glycolysis and genes encoding HLA class 2 machinery (FIG. 6F) was observed. These results revealed potential links between dysregulated EC metabolism with cytokine release syndrome and immune dysfunction in COVID-19. Network analysis demonstrated a connection of SARS-CoV-2 infection with attenuated transcriptional factor network demonstrated by downregulation of the transcriptional factors ZKSCAN1 and CSNK2B, and upregulation of KLF6, NEAT1, and JUND (FIG. 6G).

BALF ECs were next identified and subsetted for downstream analysis (FIGS. 5A-C). Differential expression analysis revealed key differences in the expression of transcripts governing key metabolic pathways (FIG. 5D). Additionally, UMAP performed solely on differentially expressed metabolic genes revealed distinct clustering of bulk epithelial cells along disease severity (FIG. 5E). Pearson correlation analysis performed on ECs revealed a strong positive correlation between Hif-1α and key glycolytic transcripts, suggesting a hypoxia-induced glycolytic metabolic reprogramming (FIG. 5F). ECs were then divided into pseudostratified ciliated and nonciliated subtypes based on the expression of canonical genes associated with cilia production (CFAP126, and DNAAF) (FIG. 5B). The ratio of pseudostratified ciliated ECs to nonciliated epithelial cells was inversely correlated with COVID-19 disease severity (FIG. 5C). This finding suggested that SARS-CoV-2 infection produced direct injury to the ciliated EC compartment. Overexpression of glycolytic transcripts (ENO1, ADHIA3, GAPDH, ALDOA, PCK2) was noted in both ciliated and nonciliated EC subsets from COVID(+) patients (FIGS. 5G, I). These results were validated by GSEA analysis, which demonstrated enrichment of glycolysis genes (FIGS. 5G, I). We also observed decreased expression of FAO regulating genes to different extents in ciliated and nonciliated ECs from severe COVID(+) compared to healthy control (FIGS. 5H, J). Hif-1α and anaerobic glycolysis gene expression was strongly correlated with reduced expression of the OXPHOS and TCA cycle genes in these EC subsets from severe COVID-19 (FIGS. 5H, J). GSEA analysis demonstrated enrichment of glycolysis, as well as a large downregulation of OXPHOS and TCA cycle regulating genes in ciliated and nonciliated ECs of severe COVID-19 patients (FIGS. 5H, J). Collectively, these results suggested that oxygen deprived conditions in the COVID-19 lung mediates a metabolic switch from aerobic FAO and OXPHOS towards anaerobic glycolysis in ECs, which is strongly linked to mitochondrial dysfunction.

B) Detailed Description of Figures from Experiment Two

FIGS. 1A-L. Distinct immunophenotype of BALFs and PBPMCs from COVID-19 patients. A. Schematic illustrating experimental design for multiparametric flow cytometry and single-cell RNA sequencing re-analysis. (B-G) The total PBMC patient cohort includes, given the high degree of lymphopenia, various subsets of the total cohort were used for different experiments. (H-I) The total BALF patient cohort includes 4 healthy, 3 moderate COVID-19, and 6 severe COVID-19 patients. One healthy patient and one severe patient were excluded from downstream analysis due to low T-cell count. B. 3-D PCA analysis conducted using bulk expression of each marker per sample as input, circles were manually drawn around the PCA plot to highlight distinct clustering. C. UMAP projection of labelled PBMC populations from 8 Healthy, 12 COVID (−), and 17 COVID (+) patients. D. Representative contoured kernel density for UMAP projection of PBMCs, a representative sample from each group was displayed. E-G. Summary graphs demonstrating frequency of CD8⁺Tc amongst all live cells in patient PBMCs (E), frequency of effector CD8Tc (grzmB+ CD8Tc) amongst all CD8+Tc (F), and frequency of CD8⁺Tc memory (CD8⁺T_{M}) amongst all CD8⁺Tc (G). H. UMAP projection displaying population labelling of 66,452 cells from healthy, moderate, and severe COVID-19⁺ patients, Tc populations were circled manually. I. UMAP projections displaying labelled unsupervised clustering analysis of 7601 reintegrated cells split between healthy, moderate, and severe COVID-19⁺ patients. (J-L). Summary graphs demonstrating frequency of bulk CD8⁺Tc amongst total BALF cells (J), effector CD8⁺Tc amongst all Tc (K), and CD8⁺Tc memory (CD8⁺T_{M}) (L) amongst all Tc. Two-tailed student's T-test was used. *p<0.05, **p<0.01, and ***p<0.001.

FIGS. 2E-K. Metabolic dysfunction is evident in CD8⁺Tc from severe COVID-19 patients. Freshly isolated PBMCs were evaluated by flow cytometry. E. Representative histogram and summary graphs demonstrating glut-1 expression in CD8⁺Tc from healthy, COVID(−), and COVID(+) patients. F. Representative histograms and summary graph demonstrating glycolytic flux of CD8⁺Tc from healthy, COVID(−), and COVID(+) patients using SCENITH. G. Representative histogram and summary graph demonstrating ΔΨm, mitochondrial membrane potential (TMRM), in CD8⁺Tc from healthy, COVID(−), and COVID(+) patients. H. Representative histograms and summary graph demonstrating mitochondrial flux of CD8⁺Tc from healthy, COVID(−), and COVID(+) patients by SCENITH assay. (I-K). Representative histogram and summary graphs demonstrating the levels of ROS (e), LC3 (J), and cpt1a (K) in CD8+Tc from healthy, COVID(−), and COVID(+) patients. In (G, H, K), three samples were excluded from analysis due to critically low CD8+Tc count; in (F), one sample was excluded from analysis due to critically low CD8+Tc count. Two-tailed student's T-test was used. *p<0.05, **p<0.01, and ***p<0.001.

FIGS. 2L-P. CD8+Tc are functionally exhausted in severe COVID-19 patients. Freshly isolated PBMCs were evaluated for metabolic properties by flow cytometry. (L-N) Representative histogram and summary graph demonstrating the abundance of lag-3+CD8+Tc (L), cellular translational level via Puromycin (M), and Ki-67 expression (N) in CD8+Tc from healthy, COVID(−), and COVID(+) patients. O. Representative histogram and paired graph abundance of Hif-1α^{High} cells in lag-3^{low} and lag-3^{high}CD8⁺Tc from COVID(+) patients. P. Representative contour plot and summary paired graph demonstrating IFNy⁺ cells in MTR^{low} and MTR^{high} CD8⁺Tc from COVID(+) patients. In (M, N), one sample was excluded due to critically low CD8⁺Tc count; in (c), three samples were excluded due to critically low CD8⁺Tc count. Two-tailed student's T-test was used. *p<0.05, **p<0.01, and ***p<0.001.

FIGS. 4A-D. Impaired memory differentiation of CD8+Tc in COVID-19. Pseudotime and trajectory inference analysis to evaluate the kinetic differentiation of CD8+Tc in the BALF during SARS-CoV-2 infection. (A-B). UMAP projection (A) and dot plot showing pseudotime values (B) of 3,694 CD8+Tc of reintegrated, healthy, moderate, or severe COVID-19 patients. C. Bar graphs displaying the frequency of CD8+Tc subpopulations across disease conditions; D. Violin plot demonstrating the expression of tissue resident memory encoding genes in CD8+TM.

FIGS. 3G-K. Metabolic reprogramming towards anaerobic glycolysis upon mitochondrial dysfunction in CD8⁺T_{M} during SARS-CoV-2 infection. Freshly isolated PBMCs were evaluated for metabolic analysis using flow cytometry. G. Representative histogram and summary graphs demonstrating glut-1 expression in CD8⁺T_{M} from healthy, COVID(−), and COVID(+) patients. H. Representative histograms and dot plot graphs demonstrating glucose dependence and FAO/AAO capacities of CD8+TM from healthy, COVID(−), and COVID(+) patients by the SCENITH assay. (I-K). Representative histogram and summary graphs demonstrating the expression of ROS (I), abundance of vdac⁺ CD8⁺Tc (J), and mitochondrial membrane potential (K) in CD8⁺T_{M} from healthy, COVID(−), and COVID(+) patients. In (H, K), three samples were excluded due to critically low CD8⁺T_{M} count; in (G, I, J), one sample was excluded due to critically low CD8⁺T_{M} count. Two-tailed student's T-test was used. *p<0.05, **p<0.01, and ***p<0.001.

FIGS. 3L-O. Metabolic dysregulation triggers functional impairment in CD8⁺T_{M}. Freshly isolated PBMCs were evaluated for cellular function and metabolic phenotype by flow cytometry. (L-M). Representative histogram and summary graphs demonstrating the expression abundance of lag-3⁺ CD8⁺T_{M} (L) and granzyme B expression (M) in CD8⁺T_{M} from healthy, COVID(−), and COVID(+) patients. N. Representative contour plot and summary paired graph demonstrating IFNy⁺ cells in MTR^{low} and MTR^{high} CD8⁺Tc from COVID(+) patients. O. Summary paired graphs demonstrating glucose dependence and FAO/AAO capacities of lag-3^{low} and lag-3^{high} CD8⁺T_{M} from COVID(+) patients. In (M, O), three samples are excluded due to critically low CD8⁺T_{M} count; in (A), one sample was excluded due to critically low CD8+TM count. Two-tailed student's T-test was used. *p<0.05, **p<0.01, and ***p<0.001.

FIGS. 3A-F. Metabolic reprogramming in BALF CD8⁺T_{M} during SARS-CoV-2 infection. A. Heatmap displaying expression of key metabolic genes of CD8⁺T_{M}. (B, C). GSEA enrichment and hierarchical clustering plot for glycolysis (B) and TCA and Respiratory Electron Transport (C) to compare severe COVID-19 vs. healthy patients. D. Linear regression and Pearson correlation analysis between module scores for glycolysis and exhaustion or FAO. E. UMAP projection of CD8⁺T_{M} clustered on 42 differentially expressed metabolic genes, circles were manually drawn to highlight clustering F. Pearson matrix showing correlation between differentially expressed genes.

FIGS. 15A-F. Mitophagy inhibition restores CD8⁺Tc and SARS-CoV-2 specific CD8⁺Tc function. PBMCs from 5 COVID-19(+) patients were activated with SARS-CoV-2 megapool CD8a peptide (see Methods) in the presence or absence of MDIVI-1 (20 μM). One sample was excluded from analysis due to critically low CD8⁺Tc count. A. Representative histogram and summary graphs demonstrating the expression of glut-1 in CD8⁺Tc from healthy and COVID(+) patients. (B-D) Representative histogram/density plots and graphs demonstrating the abundance of antigen specific (CD137⁺) (B), Ki-67⁺ (C), and IFNy⁺ (D) cells amongst CD8+Tc of vehicle or MDIVI-1 vs vehicle treated samples. (E, F) Representative histogram and summary graphs demonstrating the abundance of Ki-67 (E) and glut-1⁺ (F) cells amongst CD8⁺T_{M} of vehicle or MDIVI-1 treated samples. Two-tailed student's T-test was used. *p<0.05, **p<0.01, and ***p<0.001.

FIGS. 7F-M. Circulating NKTs exhibit dysfunctional immunometabolic phenotype in COVID-19. F. Summary graph demonstrating frequency of NKT in PBMCs from healthy, COVID(−), and COVID(+) patients. G. Summary graph demonstrating frequency of NKT in the BALF from healthy, moderate, and severe patients. H. Summary graphs and histograms demonstrating the expression of glut-1 in NKTs from healthy, COVID(−), and COVID(+) patients. I. Histograms and graphs demonstrating glycolytic flux of NKTs from COVID(−) and COVID(+) patients from the SCENITH assay. (J-M) Histograms and graphs demonstrating the expression of ROS (J), vdac (K), TMRM (L), and grzmB (M) in NKTs from healthy, COVID(−), and COVID(+) patients. In (I, K, L, M), four samples were removed from analysis due to critically low NKT cell count. Two-tailed student's T-test was used. *p<0.05, **p<0.01, and ***p<0.001.

FIG. 16. Representative flow cytometry gating strategy. Gating strategy to define population as conducted using Flowjo10.0. CD8⁺T_{M} was defined as the combination of effector memory (EM) and central memory (CM) CD8⁺Tc populations. CD8⁺T_{EM} and CD8⁺T_{CM} were defined using either CCR7 and CD62L, or CD45RA and CD62L.

FIGS. 17A-C. Unsupervised clustering for CD8⁺Tc and NK of patient PBMCS. (A) UMAP projection demonstrating unsupervised clusters generated by FlowSOM. (B) Heatmap demonstrating canonical marker expression for labelled populations identified using unsupervised clustering. (C) UMAP projection of patient PBMCs overlayed with scaled expression of markers for unsupervised clustering.

FIGS. 9A-B. Heatmap displaying expression of genes used to annotate unsupervised clusters after sample integration. A. FindAllMarkers function in Seurat was used to identify the top genes specific to each annotated cell population. CD4, macrophage, peripheral monocyte, nonciliated epithelium, B, CD8, mDC. B. pDC, proliferating T, plasma, ciliated epithelium, neutrophil, and mast cells. A heatmap displaying the average gene expression was generated using the DoHeatmap function in Seurat.

FIG. 10. Heatmap displaying canonical gene expression of genes used to annotate unsupervised clusters after Tc reintegration. FindAllMarkers function in Seurat was used to identify the top genes specific to each annotated Tc population. The average gene expression for each Tc subset was generated using the DoHeatmap function in Seurat.

FIGS. 18A-C. Association between prior comorbidities and CD8+Tc metabolic dysfunction. Criteria used to define “metabolic syndrome” are in the methodology section. A. Summary graphs demonstrating the expression of Hif-1α and glut-1 on CD8⁺Tc in patients with and without metabolic syndrome. B. Summary graphs demonstrating the expression of Hif-1α and lag-3 on CD8⁺Tc in patients with and without metabolic syndrome. C. Matrix demonstrating the Pearson correlation between parameters of elevated LDHA status, elevated CRP status, and serum glucose levels, with expression of ROS, glut-1, vdac, and lag3 in CD8⁺Tc. Two-tailed student's T-test and Pearson's correlation analysis was done for evaluation of statistical significance. *p<0.05, **p<0.01, and ***p<0.001.

FIGS. 19A-G. Abnormal metabolic phenotype of BALF effector CD8+Tc from severe COVID-19 patients. A. Heatmap displaying expression of key metabolic genes of effector CD8⁺Tc. (B, C) GSEA enrichment and hierarchical clustering plot for glycolysis (B) and TCA/respiratory electron transport (c) to compare severe COVID-19 vs. healthy control. D. Violin plot demonstrating expression of NFE2L2 and PRDX2 across disease states. E. UMAP projections unsupervised clustering of patient PBMC scRNAseq data, clusters identified by canonical marker expression to be CD8⁺Tc were circled manually and labelled. F. Heatmap displaying expression of key glycolytic genes differentially expressed in bulk CD8⁺Tc amongst healthy, mild, and severe COVID-19 patients. G. Heatmap displaying expression of key exhaustion genes differentially expressed in bulk CD8⁺Tc amongst healthy, mild, and severe COVID-19 patients.

FIGS. 13A-B. Pseudotime and trajectory inference analyses of BALF CD4⁺Tc during SARS-CoV-2 infection. (A, B). UMAP projection (A) and dot plot showing pseudotime value (B) of 2063 CD4⁺Tc from reintegrated, healthy, moderate, or severe COVID-19 patients. Each dot represents a single cell.

FIGS. 7A-C. BALF NKT are metabolically glycolytic programming in severe COVID-19. A. Heatmap displaying expression of key metabolic genes of NKT. (B, C) GSEA enrichment and hierarchical clustering plot for glycolysis (B) and TCA/respiratory electron transport (C) to compare severe COVID-19 vs. healthy control.

FIGS. 6A-G. Impaired immune surveillance and glycolysis-regulating genes in BALF ECs during SARS-CoV-2 infection. A. Heatmap displaying expression of key genes-regulating immune signaling. B. Bar graph showing GSEA analysis of immune signaling pathways, bars are colored by adjusted p value. (C, D) Linear regression and Pearson correlation analyses for glycolysis and type 1 interferon response (D) and NF-kB signaling in ECs (E). GSEA enrichment plots for “HLA class 2 antigen presentation”, “HLA class 1 antigen presentation”, “toll-like receptor cascade”, and “interferon α/β response” pathways comparing severe vs. healthy control for pseudostratified ciliated epithelial subset. (F) Linear regression and Pearson correlation analyses for glycolysis and HLA Class 2 Signaling. (G) Network based display of transcription factor-gene interaction of differentially expressed genes between severe COVID-19 and healthy patients.

FIGS. 5A-J. Metabolic reprogramming in BALF ECs in severe COVID-19. (A-B) UMAP projection of 3531 BALF ECs (A) and EC subpopulations (B) from healthy, moderate, and severe COVID-19 patients. B. UMAP projection of labelled epithelial cell subsets. C. Bar graph showing distribution of pseudostratified ciliated and nonciliated ECs. D. Heatmap displaying expression of key differentially expressed metabolic genes. E. UMAP projection of EC clustered on 42 differentially expressed metabolic genes. F. Matrix showing spearman correlation between differentially expressed metabolic genes. (G, H) Hierarchical clustering and GSEA enrichment for genes coding for glycolysis (G) and for “TCA cycle and respiratory electron transport” (H) in ciliated ECs from severe COVID-19 patients and healthy control. (I. J) Hierarchical clustering and GSEA enrichment for genes coding for glycolysis (g) and for “TCA cycle and respiratory electron transport” (H) in non-ciliated ECs from severe COVID-19 patients.

### Discussion

Metabolic comorbidities have been identified as significant risk factors for COVID-19 severity and mortality.^{5,69} However, how metabolic dysregulation in patients is linked to worsened immunopathology during SARS-CoV-2 infection remains unclear. A more comprehensive understanding of the mechanisms underlying this link would provide critical insight for the prognosis and therapeutic treatment of COVID-19. Here, using a tripartite combination of multiparametric flow cytometry, SCENITH single cell metabolomics, and scRNA-SEQ reanalysis, it is shown that immune dysregulation in SARS-CoV-2 infection is associated with metabolic reprogramming in CD8⁺Tc and NKTs, both have critical role in the anti-viral adaptive immune response.^{18,70} Notably, this metabolic dysfunction was absent in patients negative for COVID-19 who demonstrated COVID-19-like upper respiratory symptoms, validating that these altered immunometabolic profile was not merely a consequence of increased inflammation, but rather uniquely specific to SARS-CoV-2 infection.

Despite CD4⁺Tc lymphopenia having been reported in severe COVID-19 patients in a number of studies,^{70,71} it is highly controversial whether this phenomenon occurs in CD8⁺Tc. In this study, using high dimensional flow cytometry to validate and decipher highly resolved CD8⁺Tc subsets, clear, decreased cell frequency amongst CD8⁺T_{M} and NKT subsets during SARS-CoV-2 infection was demonstrated. Interestingly, an increase in the percentage of effector CD8⁺Tc in both COVID-19 patient PBMCs and severe BALFs compared to healthy individuals was observed, indicating that the initial differentiation of effector CD8₊Tc is not impaired in COVID-19. However, this is in stark contrast to the frequency of CD8⁺T_{M}, which was found to be heavily decreased compared to healthy and COVID(−) patients. Thus, these results suggest that dysfunction in both CD8⁺Tc and effector CD8⁺Tc leads to a significant impairment in CD8⁺T_{M} differentiation. Mechanistically, pseudotemporal modeling and trajectory inference analysis demonstrated that CD8⁺Tc are stalled on their differentiation trajectory towards memory cells in severe SARS-CoV-2 infection. Noticeably, CD8⁺T_{M} and NKT lymphopenia was not identified in COVID(−) patients, suggesting that reduced abundance of CD8⁺T_{M} andNKTs are specific predictive biomarkers for COVID-19.

Hyperglycemia during hospital admission is a strong predictor of COVID-19 mortality^{72,73} Consistently, increased activity of LDH, a gate-keeping glycolytic enzyme, is associated with pulmonary dysfunction during COVID-19.⁷⁴ These evidences highlight the lung dysfunction-induced hypoxia/anaerobic glycolysis axis as a key mechanism mediating dysregulated host immunometabolism during SARS-CoV-2 infection. Noticeably, Cossarizza et al. failed to detect metabolic changes in CD8⁺Tc using Seahorse bioenergetics analysis.⁷⁵ Additionally, using scRNA-SEQ and flow cytometry, the Powell group did not observe attenuation in glucose metabolism of CD8⁺Tc.⁷⁶ This discrepancy can probably be attributed to the fact that Tc were stimulated with αCD3/CD28 polyclonal activation under normoxic conditions which may result in nulling of any potential metabolic differences present in COVID-19. Another potential explanation arises from the fact that cellular metabolism in these studies was assessed for entire Tc populations, which are highly heterogeneous amongst subsets with respect to their metabolism. To this end, in the current study, using SCENITH single cell metabolomics to evaluate the bioenergetics flux of freshly isolated COVID-19⁺ PBMCs, a systematic metabolic reprogramming characterized by excessive glucose metabolism accompanied with impaired mitochondrial fitness, resulting in subsequent cellular exhaustion specific to CD8⁺T_{M} and NKT cells was elucidated. The single-cell metabolomics approach allowed for directly probing the metabolism of exhausted CD8⁺Tc and to validate a large overdependence on glucose metabolism as a hallmark of CD8⁺Tc exhaustion in COVID-19. Moreover, a strong increase in CD8⁺Tc Hif-1α expression in patients with metabolic syndrome as well as mild correlations between lag-3 and VDAC-1 expression with serum glucose level may a mechanistic justification for impaired antiviral immunity against SARS-CoV-2 infection in hyperglycemia and diabetes patients. Importantly, a metabolic shift towards increased anaerobic glycolysis occurred selectively in COVID-19 patients, compared to other COVID(−) patients with similar respiratory symptoms. Recently, Siska et al. reported that SARS-CoV-2 infection mediates increased hypoxia-induced mitochondrial ROS through the enhancement of basigin CD147 expression, resulting in mitochondrial stress and cellular dysfunction in Tc of patients with severe SARS-CoV-2.¹⁴ In contrast, the current disclosure demonstrated that augmented cytosolic ROS level was the most significant in CD8⁺Tc from COVID patients, suggesting that ROS accumulation in CD8⁺Tc is a more general feature of increased lung inflammation. Presumably, the combination of ROS accumulation with a COVID-19-specific hypoxia triggered a shift to anaerobic glycolysis, which is a primary factor driving immunometabolic dysfunction in CD8 lymphocytes during SARS-CoV-2 infection.

CD8⁺T_{M} are critical for long-term protection against viruses and strongly correlate with immune protection.⁷⁷ However, little is known about the SARS-CoV-2-specific Tc immunity in prior, virally exposed individuals, such as how CD8+TM are generated post-acute infection. Normally, CD8⁺T_{M} depend on OXPHOS and oxidation of intracellular lipids in mitochondria to sustain their energetic demands.^{78,79} However, as disclosed herein, because of reduced oxygenation due to COVID-19 lung dysfunction, both CD8⁺T_{M} and their upstream effector precursors were found to be phenotypically and functionally glycolytic, suggesting a metabolically linked impairment in CD8⁺T_{M} function and differentiation. Accordingly, CD8⁺Tc activated by SARS-CoV-2-specific peptide showed significantly upregulated glucose uptake as well as an exhausted phenotype. Given that healthy, functional SARS-CoV-2 specific CD8⁺Tc are associated with milder, recovered, and convalescent COVID-19 patients,⁸⁰ targeting the hypoxia/anaerobic glycolysis may potentially improve the function of SARS-CoV-2 antigen specific CD8⁺Tc during re-exposure to viral antigens, and rescue impaired memory cell differentiation. Supporting this hypothesis, cyclophilin A was found to restore SARS-CoV-2 specific CD8⁺Tc function through normalizing cellular metabolism.¹⁴ Further, a recent stage-2 clinical trial using 2-DG, a competitive inhibitor of glycolytic flux, as a therapeutic treatment for COVID-19 was successful in improving patient outcomes.⁸¹ The current disclosure strongly implicates mitophagy as a potential therapeutic target for COVID-19 treatment. Mitophagy is the cellular process involved in selective autophagic degradation of dysfunctional mitochondria.⁸² Depletion of impaired mitochondria via mitophagy redirects metabolism towards increased glucose utilization.^{83,84} Accordingly, ablation of mitophagy may potentially attenuate CD8⁺Tc exhaustion and improve memory cell differentiation. Indeed, pharmacological targeting of mitophagy by Mdivi-1 restored the proliferation, activation, and memory formation of CD8⁺Tc and CD8⁺T_{M}, as well as enhanced the generation of SARS-CoV-2-specific CD8⁺Tc via the attenuation of glucose metabolism. Furthermore, unlike glycolysis, mitophagy is not critical for initial Tc activation and effector cell differentiation,⁸⁵ thus, mitophagy-targeting approaches can potentially be used immediately after infection. Additionally, virus have been found to hijack intracellular mitophagy to attenuate innate immune response activity and promote viral proliferation.⁸⁶ Indeed, Mdivi-1 has been shown to effectively reduce SARS-CoV-2 replication in virally infected cells.¹⁷ Thus, therapeutic use of Mdivi-1 for COVID-19 may have a dual effect of 1) improving the efficacy of the adaptive Tc immune response via metabolic restoration and 2) directly inhibiting viral replication in the host.

NKT abundance is a strong predictive biomarker for COVID-19 outcome.⁸⁷ NKTs have been proven to have a key role in the prevention of COVID-19 induced pneumonia.¹⁹ As a key bridge between innate and adaptive immunity, it is critical to understand the function and role of NKTs during SARS-CoV-2 infection. However, little is known about NKT metabolism during infection. Consistently, in current the study, NKT lymphopenia was evident in both the BALFs and PBMCs. Furthermore, COVID-19(+) NKTs demonstrated significantly increased hypoxia-mediated anaerobic glycolytic activity, accompanied by elevated mitochondrial impairment. However, unlike CD8⁺Tc, a strong increase in OXPHOS was seen in NKTS from severe COVID-19 patients. This differential metabolic response can be explained by the observations that NKT cells are considerably more dependent on mitochondrial metabolism after activation in comparison to conventional Tc.^{21,22} Whereas conventional Tc exhibit a tremendous Warburg-like upregulation of aerobic glycolysis upon activation,⁸⁸ NKTs remain heavily reliant on mitochondrial respiration.^{21,22} Thus, upon the onset of systemic hypoxia after initial lymphocyte activation, OXPHOS-dependent NKTs are likely unable to sustain mitochondrial activity and thus also upregulate glycolysis to sustain their bioenergetics demands. These results thus highlight that this metabolic reprogramming is associated with a decrease in the frequency of NKTs, as well as an impairment in cytolytic function.

Interestingly, it was found that SARA-CoV-2 derived EC damage creates oxygen-deprived conditions in the lungs that not only induce metabolic reprogramming of various immune cell subsets, but also themselves. It was found that during COVID-19 infection, differential metabolism drives lung ECs towards senescence and towards acquiring a significant SASP phenotype, leading to secretion of proinflammatory cytokines, reduced HLA class 2 mediated immunosurveillance, and increased HLA class 1 machinery. Prolonged stimulation of exhausted lymphocytes, that demonstrate attenuated effector function and cytokine secretion in nutrient-depleted microenvironments, by antigen presenting cells via HLA class 1 leads to significantly increased cellular exhaustion,⁸⁹ which further impairs the capacity of cells to differentiate into memory phenotypes. These results therefore show that the immunometabolic rewiring of ECs in the BALFs can be a potential mechanism for organ-specific lymphocyte exhaustion and memory cell dysfunction. Further, this observation thus highlights that unconventional antigen presentation on non-hematopoietic ECs via HLA class 1, in addition to conventional antigen presentation by professional APCs (monocyte, DC, and macrophage), can be considered as a potential target for therapeutic development.

In summary, it is shown that CD8⁺Tc, NKTs, and ECs undergo a global metabolic reprogramming towards anaerobic metabolic processes including glycolysis, mitophagy, and glutaminolysis. As a result, specific CD8⁺Tc subsets and NKTs demonstrate significant metabolically-linked exhaustion and effector function, as well as impaired differentiation into memory cells. It further validates mitophagy as a potential target for therapeutic treatment of severe SARS-CoV-2. This disclosure therefore sheds important new light on the key molecular and cellular mechanisms by which immunometabolism regulates pathobiology in SARS-CoV-2 infection and validate the concept of targeting immunometabolism to treat acute COVID-19 severity or to enhance the efficacy of COVID-19 vaccination therapies.

### Conclusion of Application

Overall, these studies shed important new light on the molecular and cellular mechanisms by which immune cell metabolism regulates COVID-19 pathobiology. These studies provide crucial information about biomarkers, therapeutic targets, and strategies for the COVID-19 therapy. A novel data analysis pipeline for understanding single cell metabolism in an organ-specific manner is also provided by these studies. With the rapid development of single-cell omics techniques, this comprehensive portrait of metabolic features of immune cells will advance COVID-19 research and help devise novel approaches to mitigate the COVID-19 pandemic.

All patents and publications mentioned in this specification are indicative of the levels of those skilled in the art to which the invention pertains. All patents and publications are herein incorporated by reference to the same extent as if each individual publication was specifically and individually indicated to be incorporated by reference. It is to be understood that while a certain form of the invention is illustrated, it is not intended to be limited to the specific form or arrangement herein described and shown. It will be apparent to those skilled in the art that various changes may be made without departing from the scope of the invention and the invention is not to be considered limited to what is shown and described in the specification. One skilled in the art will readily appreciate that the present invention is well adapted to carry out the objectives and obtain the ends and advantages mentioned, as well as those inherent therein. The assays, biomarkers, methods, procedures, elements, and compositions described herein are presently representative of the preferred embodiments, are intended to be exemplary and are not intended as limitations on the scope. Changes therein and other uses will occur to those skilled in the art which are encompassed within the spirit of the invention. Although the invention has been described in connection with specific, preferred embodiments, it should be understood that the invention as ultimately claimed should not be unduly limited to such specific embodiments. Indeed various modifications of the described modes for carrying out the invention which are obvious to those skilled in the art are intended to be within the scope of the invention.

### References for Experiments 1 and 2

1 Zhu, N. et al. A Novel Coronavirus from Patients with Pneumonia in China, 2019. N Engl J Med 382, 727-733, doi:10.1056/NEJMoa2001017 (2020).
2 Team, I. C.-F. Modeling COVID-19 scenarios for the United States. Nat Med, doi:10.1038/s41591-020-1132-9 (2020).
3 Madhi, S. A. et al. Efficacy of the ChAdOx1 nCoV-19 Covid-19 Vaccine against the B.1.351 Variant. N Engl J Med, doi:10.1056/NEJMoa2102214 (2021).
4 Song, J. W. et al. Omics-Driven Systems Interrogation of Metabolic Dysregulation in COVID-19 Pathogenesis. Cell Metab, doi:10.1016/j.cmet.2020.06.016 (2020).
5 Codo, A. C. et al. Elevated Glucose Levels Favor SARS-CoV-2 Infection and Monocyte Response through a HIF-1alpha/Glycolysis-Dependent Axis. Cell Metab, doi:10.1016/j.cmet.2020.07.007 (2020).
6 Xie, J. et al. Metabolic Syndrome and COVID-19 Mortality Among Adult Black Patients in New Orleans. Diabetes Care, doi:10.2337/dc20-1714 (2020).
7 Wu, D. et al. Plasma metabolomic and lipidomic alterations associated with COVID-19. Natl Sci Rev 7, 1157-1168, doi:10.1093/nsr/nwaa086 (2020).
8 Siska, P. J. et al. Metabolic stress and disease-stage specific basigin expression of peripheral blood immune cell subsets in COVID-19 patients. medRxiv, 2020.2009.2018.20194175, doi:10.1101/2020.09.18.20194175 (2020).
9 Lim, A. R., Rathmell, W. K. & Rathmell, J. C. The tumor microenvironment as a metabolic barrier to effector T cells and immunotherapy. Elife 9, doi:10.7554/eLife.55185 (2020).
10 Zhou, F. et al. Clinical course and risk factors for mortality of adult inpatients with COVID-19 in Wuhan, China: a retrospective cohort study. Lancet 395, 1054-1062, doi:10.1016/50140-6736(20)30566-3 (2020).
11 Huang, C. et al. Clinical features of patients infected with 2019 novel coronavirus in Wuhan, China. Lancet 395, 497-506, doi:10.1016/S0140-6736(20)30183-5 (2020).
12 Zhang, W. et al. Lactate Is a Natural Suppressor of RLR Signaling by Targeting MAVS. Cell 178, 176-189 e115, doi:10.1016/j.cell.2019.05.003 (2019).
13 Zheng, H. Y. et al. Elevated exhaustion levels and reduced functional diversity of T cells in peripheral blood may predict severe progression in COVID-19 patients. Cell Mol Immunol 17, 541-543, doi:10.1038/s41423-020-0401-3 (2020).
14 Nguyen, H. D., Kuril, S., Bastian, D. & Yu, X. Z. T-Cell Metabolism in Hematopoietic Cell Transplantation. Front Immunol 9, 176, doi:10.3389/fimmu.2018.00176 (2018).
15 Kuba, K. et al. A crucial role of angiotensin converting enzyme 2 (ACE2) in SARS coronavirus-induced lung injury. Nat Med 11, 875-879, doi:10.1038/nm1267 (2005).
16 Li, X. C. et al. Risk factors for severity and mortality in adult COVID-19 inpatients in Wuhan. J Allergy Clin Immun 146, 110-118, doi:10.1016/j.jaci.2020.04.006 (2020).
17 Han, Y. et al. Lactate dehydrogenase, an independent risk factor of severe COVID-19 patients: a retrospective and observational study. Aging (Albany NY) 12, 11245-11258, doi:10.18632/aging.103372 (2020).
18 Haas, R. et al. Lactate Regulates Metabolic and Pro-inflammatory Circuits in Control of T Cell Migration and Effector Functions. PLoS Biol 13, e1002202, doi:10.1371/journal.pbio.1002202 (2015).
19 Singh, J., Rahman, S. A., Ehtesham, N. Z., Hira, S. & Hasnain, S. E. SARS-CoV-2 variants of concern are emerging in India. Nat Med, doi:10.1038/s41591-021-01397-4 (2021).
20 Liao, M. et al. Single-cell landscape of bronchoalveolar immune cells in patients with COVID-19. Nat Med, doi:10.1038/s41591-020-0901-9 (2020).
21 Vabret, N. et al. Immunology of COVID-19: Current State of the Science. Immunity 52, 910-941, doi:10.1016/j.immuni.2020.05.002 (2020).
22 Liao, M. et al. Single-cell landscape of bronchoalveolar immune cells in patients with COVID-19. Nat Med 26, 842-844, doi:10.1038/s41591-020-0901-9 (2020).
23 Maucourant, C. et al. Natural killer cell immunotypes related to COVID-19 disease severity. Sci Immunol 5, doi:10.1126/sciimmunol.abd6832 (2020).
24 Westmeier, J. et al. Impaired Cytotoxic CD8(+) T Cell Response in Elderly COVID-19 Patients. mBio 11, doi:10.1128/mBio.02243-20 (2020).
25 Van Gassen, S., Gaudilliere, B., Angst, M. S., Saeys, Y. & Aghaeepour, N. CytoNorm: A Normalization Algorithm for Cytometry Data. Cytometry A 97, 268-278, doi:10.1002/cyto.a.23904 (2020).
26 Van Gassen, S. et al. FlowSOM: Using self-organizing maps for visualization and interpretation of cytometry data. Cytometry A 87, 636-645, doi:10.1002/cyto.a.22625 (2015).
27 Zhang, X. et al. Viral and host factors related to the clinical outcome of COVID-19. Nature, doi:10.1038/s41586-020-2355-0 (2020).
28 Waickman, A. T. & Powell, J. D. mTOR, metabolism, and the regulation of T-cell differentiation and function. Immunol Rev 249, 43-58, doi:10.1111/j.1600-065X.2012.01152.x (2012).
29 Tullius, S. G. et al. NAD+ protects against EAE by regulating CD4+ T-cell differentiation. Nat Commun 5, 5101, doi:10.1038/ncomms6101 (2014).
30 Chatterjee, S. et al. CD38-NAD(+)Axis Regulates Immunotherapeutic Anti-Tumor T Cell Response. Cell Metab 27, 85-100 e108, doi:10.1016/j.cmet.2017.10.006 (2018).
31 Macintyre, A. N. et al. The glucose transporter Glut1 is selectively essential for CD4 T cell activation and effector function. Cell Metab 20, 61-72, doi:10.1016/j.cmet.2014.05.004 (2014).
32 Kim, J. et al. VDAC oligomers form mitochondrial pores to release mtDNA fragments and promote lupus-like disease. Science 366, 1531-1536, doi: 10.1126/science.aav4011 (2019).
33 Pollizzi, K. N. & Powell, J. D. Regulation of T cells by mTOR: the known knowns and the known unknowns. Trends Immunol 36, 13-20, doi:10.1016/j.it.2014.11.005 (2015).
34 DiPiazza, A. T., Graham, B. S. & Ruckwardt, T. J. T cell immunity to SARS-CoV-2 following natural infection and vaccination. Biochem Biophys Res Commun, doi:10.1016/j.bbrc.2020.10.060 (2020).
35 Angin, M. et al. Metabolic plasticity of HIV-specific CD8(+) T cells is associated with enhanced antiviral potential and natural control of HIV-1 infection. Nat Metab 1, 704-716, doi:10.1038/s42255-019-0081-4 (2019).
36 Nguyen, H. et al. Complement C3a and C5a receptors promote GVHD by suppressing mitophagy in recipient dendritic cells. JCI Insight 3, doi:10.1172/jci.insight.121697 (2018).
37 Peng, Y. et al. Broad and strong memory CD4(+) and CD8(+) T cells induced by SARS-CoV-2 in UK convalescent individuals following COVID-19. Nat Immunol, doi:10.1038/s41590-020-0782-6 (2020).
38 Unsoeld, H. & Pircher, H. Complex memory T-cell phenotypes revealed by coexpression of CD62L and CCR7. J Virol 79, 4510-4513, doi:10.1128/JVI.79.7.4510-4513.2005 (2005).
39 Singh, Y. et al. SARS-CoV-2 infection paralyzes cytotoxic and metabolic functions of immune cells. bioRxiv, 2020.2009.2004.282780, doi:10.1101/2020.09.04.282780 (2020).
40 Schenkel, J. M. & Masopust, D. Tissue-resident memory T cells. Immunity 41, 886-897, doi:10.1016/j.immuni.2014.12.007 (2014).
41 Wen, W. et al. Immune cell profiling of COVID-19 patients in the recovery stage by single-cell sequencing. Cell Discov 6, 31, doi:10.1038/s41421-020-0168-9 (2020).
42 Zamorano Cuervo, N. & Grandvaux, N. ACE2: Evidence of role as entry receptor for SARS-CoV-2 and implications in comorbidities. Elife 9, doi: 10.7554/eLife.61390 (2020).
43 Xu, J. et al. SARS-CoV-2 induces transcriptional signatures in human lung epithelial cells that promote lung fibrosis. Respir Res 21, 182, doi:10.1186/s12931-020-01445-6 (2020).
44 Bonelli, M. et al. IRF1 is critical for the TNF-driven interferon response in rheumatoid fibroblast-like synoviocytes: JAKinibs suppress the interferon response in RA-FLSs. Exp Mol Med 51, 75, doi:10.1038/s12276-019-0267-6 (2019).
45 Salminen, A., Kauppinen, A. & Kaarniranta, K. Emerging role of NF-kappaB signaling in the induction of senescence-associated secretory phenotype (SASP). Cell Signal 24, 835-845, doi:10.1016/j.cellsig.2011.12.006 (2012).
46 Certo, M., Tsai, C. H., Pucino, V., Ho, P. C. & Mauro, C. Lactate modulation of immune responses in inflammatory versus tumour microenvironments. Nat Rev Immunol, doi:10.1038/s41577-020-0406-2 (2020).
47 McLane, L. M., Abdel-Hakeem, M. S. & Wherry, E. J. CD8 T Cell Exhaustion During Chronic Viral Infection and Cancer. Annu Rev Immunol 37, 457-495, doi:10.1146/annurev-immunol-041015-055318 (2019).
48 Cascone, T. et al. Increased Tumor Glycolysis Characterizes Immune Resistance to Adoptive T Cell Therapy. Cell Metab 27, 977-987 e974, doi:10.1016/j.cmet.2018.02.024 (2018).
49 van Hall, T. et al. Monalizumab: inhibiting the novel immune checkpoint NKG2A. J Immunother Cancer 7, 263, doi:10.1186/s40425-019-0761-3 (2019).
50 Borst, L., van der Burg, S. H. & van Hall, T. The NKG2A-HLA-E Axis as a Novel Checkpoint in the Tumor Microenvironment. Clin Cancer Res 26, 5549-5556, doi:10.1158/1078-0432.CCR-19-2095 (2020).
51 Schousboe, P. et al. Assessment of pulmonary surfactant in COVID-19 patients. Crit Care 24, 552, doi:10.1186/s13054-020-03268-9 (2020).
52 Moore, B. J. B. & June, C. H. Cytokine release syndrome in severe COVID-19. Science, doi:10.1126/science.abb8925 (2020).
53 Paget, C. & Trottein, F. Role of type 1 natural killer T cells in pulmonary immunity. Mucosal Immunol 6, 1054-1067, doi:10.1038/mi.2013.59 (2013).
54 Everts, B. et al. TLR-driven early glycolytic reprogramming via the kinases TBK1-IKKvarepsilon supports the anabolic demands of dendritic cell activation. Nat Immunol 15, 323-332, doi:10.1038/ni.2833 (2014).
55 Nabekura, T. et al. Cutting Edge: NKG2D Signaling Enhances NK Cell Responses but Alone Is Insufficient To Drive Expansion during Mouse Cytomegalovirus Infection. J Immunol 199, 1567-1571, doi:10.4049/jimmunol.1700799 (2017).
56 Pizzolato, G. et al. Single-cell RNA sequencing unveils the shared and the distinct cytotoxic hallmarks of human TCRVdelta1 and TCRVdelta2 gammadelta T lymphocytes. Proc Natl Acad Sci USA 116, 11906-11915, doi:10.1073/pnas.1818488116 (2019).
57 Juelke, K. et al. CD62L expression identifies a unique subset of polyfunctional CD56dim NK cells. Blood 116, 1299-1307, doi:10.1182/blood-2009-11-253286 (2010).
58 Harvey, W. T. et al. SARS-CoV-2 variants, spike mutations and immune escape. Nat Rev Microbiol, doi:10.1038/s41579-021-00573-0 (2021).
59 Hariyanto, T. I. & Kurniawan, A. Metformin use is associated with reduced mortality rate from coronavirus disease 2019 (COVID-19) infection. Obes Med 19, 100290, doi:10.1016/j.obmed.2020.100290 (2020).
60 Ayres, J. S. A metabolic handbook for the COVID-19 pandemic. Nat Metab 2, 572-585, doi:10.1038/s42255-020-0237-2 (2020).
61 De Biasi, S. et al. Marked T cell activation, senescence, exhaustion and skewing towards TH17 in patients with COVID-19 pneumonia. Nat Commun 11, 3434, doi:10.1038/s41467-020-17292-4(2020).
62 Drosten, C. et al. Identification of a novel coronavirus in patients with severe acute respiratory syndrome. N Engl J Med 348, 1967-1976, doi:10.1056/NEJMoa030747 (2003).
63 Xu, G. et al. The differential immune responses to COVID-19 in peripheral and lung revealed by single-cell RNA sequencing. Cell Discov 6, 73, doi:10.1038/s41421-020-00225-2 (2020).
64 Ferretti, A. P. et al. Unbiased Screens Show CD8(+) T Cells of COVID-19 Patients Recognize Shared Epitopes in SARS-CoV-2 that Largely Reside outside the Spike Protein. Immunity 53, 1095-1107 e1093, doi:10.1016/j.immuni.2020.10.006 (2020).
65 Thompson, E. et al. Mitochondrial induced T cell apoptosis and aberrant myeloid metabolic programs define distinct immune cell subsets during acute and recovered SARS-CoV-2 infection. medRxiv, 2020.2009.2010.20186064, doi:10.1101/2020.09.10.20186064 (2020).
66 Kusnadi, A. et al. Severely ill COVID-19 patients display augmented functional properties in SARS-CoV-2-reactive CD8<sub>+</sub> T cells. bioRxiv, 2020.2007.2009.194027, doi:10.1101/2020.07.09.194027 (2020).
67 Thompson, E. A. et al. Metabolic programs define dysfunctional immune responses in severe COVID-19 patients. medRxiv, 2020.2009.2010.20186064, doi:10.1101/2020.09.10.20186064 (2020).
68 Group, R. C. et al. Dexamethasone in Hospitalized Patients with Covid-19-Preliminary Report. N Engl J Med, doi:10.1056/NEJMoa2021436 (2020).
69 Ma, R. et al. Switch of glycolysis to gluconeogenesis by dexamethasone for treatment of hepatocarcinoma. Nat Commun 4, 2508, doi:10.1038/ncomms3508 (2013).
70 Cron, R. Q. COVID-19 cytokine storm: targeting the appropriate cytokine. Lancet Rheumatol 3, e236-e237, doi:10.1016/52665-9913(21)00011-4 (2021).
71 Wu, D. et al. Type 1 Interferons Induce Changes in Core Metabolism that Are Critical for Immune Function. Immunity 44, 1325-1336, doi:10.1016/j.immuni.2016.06.006 (2016).
72 Park, A. & Iwasaki, A. Type I and Type III Interferons—Induction, Signaling, Evasion, and Application to Combat COVID-19. Cell Host Microbe 27, 870-878, doi:10.1016/j.chom.2020.05.008 (2020).
73 Yu, Y. R. et al. Disturbed mitochondrial dynamics in CD8(+) TILs reinforce T cell exhaustion. Nat Immunol 21, 1540-1551, doi:10.1038/s41590-020-0793-3 (2020).
74 Xu, R., Ji, Z., Xu, C. & Zhu, J. The clinical value of using chloroquine or hydroxychloroquine as autophagy inhibitors in the treatment of cancers: A systematic review and meta-analysis. Medicine (Baltimore) 97, e12912, doi:10.1097/MD.0000000000012912 (2018).
75 Boulware, D. R. et al. A Randomized Trial of Hydroxychloroquine as Postexposure Prophylaxis for Covid-19. N Engl J Med 383, 517-525, doi:10.1056/NEJMoa2016638 (2020).
76 Self, W. H. et al. Effect of Hydroxychloroquine on Clinical Status at 14 Days in Hospitalized Patients With COVID-19: A Randomized Clinical Trial. JAMA 324, 2165-2176, doi:10.1001/jama.2020.22240 (2020).
77 Gupta, S. S. et al. NIX-Mediated Mitophagy Promotes Effector Memory Formation in Antigen-Specific CD8(+) T Cells. Cell Rep 29, 1862-1877 e1867, doi:10.1016/j.celrep.2019.10.032 (2019).
78 Ziegler, P. K. et al. Mitophagy in Intestinal Epithelial Cells Triggers Adaptive Immunity during Tumorigenesis. Cell 174, 88-101 e116, doi:10.1016/j.cell.2018.05.028 (2018).
79 Xie, J. et al. Association Between Hypoxemia and Mortality in Patients With COVID-19. Mayo Clin Proc 95, 1138-1147, doi:10.1016/j.mayocp.2020.04.006 (2020).
80 Tillett, R. L. et al. Genomic evidence for reinfection with SARS-CoV-2: a case study. Lancet Infect Dis 21, 52-58, doi:10.1016/S1473-3099(20)30764-7 (2021).
81 Egawa, T. & Bhattacharya, D. Regulation of metabolic supply and demand during B cell activation and subsequent differentiation. Curr Opin Immunol 57, 8-14, doi:10.1016/j.coi.2018.10.003 (2019).
82 Kaneko, N. et al. Loss of Bel-6-Expressing T Follicular Helper Cells and Germinal Centers in COVID-19. Cell 183, 143-157 e113, doi:10.1016/j.cell.2020.08.025 (2020).
83 Woodruff, M. C. et al. Extrafollicular B cell responses correlate with neutralizing antibodies and morbidity in COVID-19. Nat Immunol 21, 1506-1516, doi:10.1038/s41590-020-00814-z (2020).
84 Dan, J. M. et al. Immunological memory to SARS-CoV-2 assessed for up to 8 months after infection. Science 371, doi:10.1126/science.abf4063 (2021).
85 Gaebler, C. et al. Evolution of Antibody Immunity to SARS-CoV-2. bioRxiv, 2020.2011.2003.367391, doi:10.1101/2020.11.03.367391 (2020).
86 Zhu, L. et al. Single-Cell Sequencing of Peripheral Mononuclear Cells Reveals Distinct Immune Response Landscapes of COVID-19 and Influenza Patients. Immunity 53, 685-696 e683, doi:10.1016/j.immuni.2020.07.009 (2020).
87 Marongiu, L. et al. Maturation signatures of conventional dendritic cell subtypes in COVID-19 reflect direct viral sensing. bioRxiv, 2021.2003.2003.433597, doi:10.1101/2021.03.03.433597 (2021).
88 Jouan, Y. et al. Phenotypical and functional alteration of unconventional T cells in severe COVID-19 patients. J Exp Med 217, doi:10.1084/jem.20200872 (2020).
89 Cerwenka, A. & Lanier, L. L. Natural killer cell memory in infection, inflammation and cancer. Nat Rev Immunol 16, 112-123, doi:10.1038/nri.2015.9 (2016).
90 Zhang, J. Z., Behrooz, A. & Ismail-Beigi, F. Regulation of glucose transport by hypoxia. Am J Kidney Dis 34, 189-202, doi:10.1016/s0272-6386(99)70131-9 (1999).
91 Lucas, C. et al. Longitudinal analyses reveal immunological misfiring in severe COVID-19. Nature, doi:10.1038/s41586-020-2588-y (2020).
92 Nightingale Health, U. K. B. I., Julkunen, H., Cichonska, A., Slagboom, P. E. & Wurtz, P. Metabolic biomarker profiling for identification of susceptibility to severe pneumonia and COVID-19 in the general population. Elife 10, doi:10.7554/eLife.63033 (2021).
93 Codo, A. C. et al. Elevated Glucose Levels Favor SARS-CoV-2 Infection and Monocyte Response through a HIF-1alpha/Glycolysis-Dependent Axis. Cell Metab 32, 437-446 e435, doi:10.1016/j.cmet.2020.07.007 (2020).
94 Abdool Karim, S. S. & de Oliveira, T. New SARS-CoV-2 Variants—Clinical, Public Health, and Vaccine Implications. N Engl J Med 384, 1866-1868, doi:10.1056/NEJMc2100362 (2021).
95 Hahne, F. et al. flowCore: a Bioconductor package for high throughput flow cytometry. BMC Bioinformatics 10, 106, doi:10.1186/1471-2105-10-106 (2009).
96 Finak, G. et al. OpenCyto: an open source infrastructure for scalable, robust, reproducible, and automated, end-to-end flow cytometry data analysis. PLoS Comput Biol 10, e1003806, doi:10.1371/journal.pcbi.1003806 (2014).
97 Chevrier, S. et al. Compensation of Signal Spillover in Suspension and Imaging Mass Cytometry. Cell Syst 6, 612-620 e615, doi:10.1016/j.cels.2018.02.010 (2018).
98 Morse, C. et al. Proliferating SPP1/MERTK-expressing macrophages in idiopathic pulmonary fibrosis. Eur Respir J 54, doi:10.1183/13993003.02441-2018 (2019).
99 Butler, A., Hoffman, P., Smibert, P., Papalexi, E. & Satija, R. Integrating single-cell transcriptomic data across different conditions, technologies, and species. Nat Biotechnol 36, 411-+, doi:10.1038/nbt.4096 (2018).
100 Stuart, T. et al. Comprehensive Integration of Single-Cell Data. Cell 177, 1888-+, doi:10.1016/j.cell.2019.05.031 (2019).
101 Cao, J. et al. The single-cell transcriptional landscape of mammalian organogenesis. Nature 566, 496-502, doi:10.1038/s41586-019-0969-x (2019).
102 Zhou, G. Y. et al. NetworkAnalyst 3.0: a visual analytics platform for comprehensive gene expression profiling and meta-analysis. Nucleic Acids Res 47, W234-W241, doi:10.1093/nar/gkz240 (2019).
103 Yu, G. Using ggtree to Visualize Data on Tree-Like Structures. Curr Protoc Bioinformatics 69, e96, doi:10.1002/cpbi.96 (2020).
104 Mathewson, N. D. et al. Corrigendum: Gut microbiome-derived metabolites modulate intestinal epithelial cell damage and mitigate graft-versus-host disease. Nat Immunol 17, 1235, doi:10.1038/ni1016-1235b (2016).
105 Kuleshov, M. V. et al. Enrichr: a comprehensive gene set enrichment analysis web server 2016 update. Nucleic Acids Res 44, W90-97, doi:10.1093/nar/gkw377 (2016).
106 Karagiannis, F. et al. Impaired ketogenesis ties metabolism to T cell dysfunction in COVID-19. Nature 609, 801-807, doi:10.1038/s41586-022-05128-8 (2022).

### SUPPLEMENTARY REFERENCES

Cervia, Carlos et al. Immunoglobulin signature predicts risk of post-acute COVID-19 syndrome. Nature Communications 13:446 2022.
Hadjadj, Samy et al. Glucose-lowering treatments and COVID-19 morality in T2DM. Nature Reviews Endocrinolog 17(7):387-388 2021.
Khunti, Kamlesh et al. Prescription of glucose-lowering therapies and risk of COVID-19 mortality in people with type 2 diabetes: a nationwide observational study in England. Lancet Diabetes Endocrinolog 9(5):293-303 2021.

TABLE 9
Chemical Table, and Peptide Table

| | | |
|---|---|---|
| Reagent/Resource | Source | Identifier |

Antibodies

| | | |
|---|---|---|
| Anti-Human CD8 APC/Cy7 | BioLegend | 344722 |
| Anti-Human CD4 PECy7 | BioLegend | 300512 |
| Anti-Human CD137 PE | BioLegend | 309804 |
| Anti-Human LAG-3 PE-Dazzle | BioLegend | 369332 |
| Anti-Human CD45RA PerCP | BioLegend | 304156 |
| Anti-Human GLUT1 AF700 | R&D Systems | FAB1418N |
| Anti-Human CD62L BV510 | BioLegend | 304844 |
| Anti-Human CD56 BV650 | BioLegend | 362532 |
| Anti-Mouse/Human Ki67 PE-Dazzle | BioLegend | 151220 |
| Anti-Mouse/Human Granzyme B PECy7 | BioLegend | 372214 |
| Anti-Mouse/Human LC3 | BioLegend | 848802 |
| Anti-Human CD4 Pacific Blue | BioLegend | 300521 |
| Anti-Human CD98 FITC | BioLegend | 315603 |
| Anti-Human IFNy PECy7 | BioLegend | 502528 |
| Anti-Human TNF-alpha APC | BioLegend | 502912 |
| Anti-Mouse/Human CPT1a | Santa Cruz | sc-393070 |
| | Biotechnology | |
| Anti-Human CD8 FITC | BioLegend | 980908 |
| Anti-Human HIF-1-alpha APC | R&D Systems | IC1935A |
| Anti-Mouse/Human VDAC | Alomone Labs | AVC-001 |
| Anti-Human CCR7 BV510 | BioLegend | 353232 |
| Anti-Human H2DCFDA | ThermoFisher | D399 |
| Anti-Rabbit Antibody FITC | Southern Biotechnology | 4030-02 |
| | Associates | |
| Anti-Mouse Antibody FITC | Invitrogen | F-2761 |

Chemicals

| | | |
|---|---|---|
| PBS | Fisher Scientific | BP3994 |
| DMSO | Thermo Scientific | 036480.K2 |
| 2-DG | Acros Organics | 1.12E+08 |
| Oligomycin A | Cayman | 11342 |
| Mdivi-1 | Cayman | 15559 |
| Fetal Bovine Serum, Heat-inactivated | Sigma | F4135-500ML |
| Sodium Azide | Acros Organics | 19038-1000 |
| BSA | GoldBio | A-420-100 |
| FAM-Dc-Puromycin | Jena Bioscience | NU-925-6FM-S |
| 2-NBDG | Invitrogen | N13195 |
| TMRM | ThermoFisher | T668 |
| Mitotracker | Cell Signaling | 9082 |
| BODIPY | Invitrogen | C2102 |
| Live/Dead | Invitrogen | L34959 |
| PMA | Sigma | P8139 |
| Ionomycin calcium salt from | Sigma | I0634 |
| Streptomyces conglobatus | | |
| FOXP3 Fix/Perm Buffer | BioLegend | 421401 |
| Puromycin | Cayman | 13884 |
| Anti-Puromycin AF647 | EMD Millipore | MABE343- |
| | | AF647 |
| RPMI 1640 Medium | Gibco | 11875-093 |

Peptides

| | | |
|---|---|---|
| SARS-Cov-2 CD8 epitope megapool | Drs. Alessandro Sette | Megapool A⁹² |
| | & Weiskopf | |

What is claimed is:

1. A method for predicting severity of COVID-19 disease in a patient having or suspected of having COVID-19 disease, the method comprising:
obtaining a sample of bronchoalveolar lavage fluid (BALF) from the patient;
performing a single cell transcriptomic analysis of the BALF obtained from the patient;
obtaining a sample of bronchoalveolar lavage fluid (BALF) from a healthy subject;
performing a single cell transcriptomic analysis of the BALF obtained from the healthy subject;
comparing the single cell transcriptomic analysis obtained from the patient to the single cell transcriptomic analysis obtained from the healthy subject and identifying differences from the comparison which indicate biomarkers of the severity of COVID-19 disease;
using the biomarkers indicated to predict the severity of COVID-19 disease in the patient;
obtaining a blood sample from the patient to isolate peripheral blood mononuclear cells (PBMCs) from the blood sample;
performing high-dimensional immune profiling of the PBMCs obtained from the patient to identify and characterize the populations of lymphocytes contained therein;
obtaining a blood sample from the healthy subject to isolate the PBMCs from the blood sample;
performing high-dimensional immune profiling of the PBMCs obtained from the healthy subject to identify and characterize the populations of lymphocytes contained therein;
comparing the populations of lymphocytes identified from the patient to the populations of lymphocytes identified from the healthy subject and determining differences from the comparison which indicate biomarkers of the severity of COVID-19 disease; and
using the biomarkers indicated to predict the severity of COVID-19 disease in the patient.

2. The method according to claim 1, wherein performing the single cell transcriptomic analysis of the BALF includes:
quality control and batch effect correction;
unsupervised clustering and non-linear dimensionality reduction for high-dimensional immunophenotyping;
differential expression and abundance analysis; and
nonparametric correlation analysis.

3. The method according to claim 1, wherein obtaining the blood sample includes isolating the PBMCs from the whole blood using density dependent centrifugation.

4. The method according to claim 1, wherein the high-dimensional immune profiling is carried out using flow cytometry.

5. The method according to claim 1, wherein the biomarkers indicated include GLUT1⁺ VDAC⁺ CD8 cells, GLUT1⁺ VDAC⁺ CD8 NKT cells, and GLUT1⁺ CD62L⁺ NK cells.

6. The method according to claim 1, further including obtaining a sample of bronchoalveolar lavage fluid (BALF) from a patient having a non-COVID respiratory infection and carrying out the method on the sample.

7. The method according to claim 1, further comprising carrying out single-cell metabolomics on lymphocytes identified from the population of lymphocytes contained within the PBMCs obtained from the patient and on lymphocytes identified from the population of lymphocytes contained within the PBMCs obtained from the healthy subject.

8. The method according to claim 7, wherein carrying out single-cell metabolomics includes carrying out a SCENITH metabolomics assay.

9. The method according to claim 8, wherein carrying out the SCENITH metabolomics assay includes carrying out a glucose uptake assay.

10. The method according to claim 7, wherein the identified lymphocytes are at least one of CD8 cells and Natural Killer T cells (NKTs).

11. The method according to claim 10, wherein the CD8 cells are CD8 memory cells.

12. The method according to claim 7, further comprising carrying out scRNA-SEQ reanalysis.

13. A biomarker for predicting severity of COVID-19 disease in a patient having COVID-19 disease comprising a metabolic profile of a CD8 cell.

14. The biomarker according to claim 13, wherein the CD8 cell is a CD8 memory cell.

15. A method for inhibiting mitophagy in cells isolated from bronchoalveolar lavage fluid (BALF), the method comprising:
providing a composition including Mdivi-1; and
administering the composition to the cells isolated from BALF.

16. The method according to claim 15, wherein the cells isolated from BALF are at least one of lymphocytes and epithelial cells (ECs).

17. The method according to claim 15, wherein the bronchoalveolar lavage fluid (BALF) is obtained from at least one of a patient having or suspected of having COVID-19 disease, a healthy patient, and a patient having a non-COVID respiratory disease.

18. A method for restoring lymphocyte function in lymphocytes isolated from a patient having COVID-19 disease, the method comprising:
providing a composition including Mdivi-1; and
administering the composition to the lymphocytes isolated from a patient having COVID-19 disease.

19. The method according to claim 18, wherein the lymphocytes are at least one of CD8⁺T_{C} and CD8⁺T_{M}.

20. A method for restoring proliferation, activation, and memory formation of CD8⁺T_{C} and CD8⁺T_{M} isolated from a patient having COVID-19 disease, the method comprising:
providing a composition including Mdivi-1; and
administering the composition to the CD8+Tc and CD8+TM isolated from a patient having COVID-19 disease.

21. A method for inhibiting mitophagy in lymphocytes of a patient having or suspected of having COVID-19 disease, the method comprising:
providing a composition including Mdivi-1; and
administering the composition to the lymphocytes of the patient.

22. The method according to claim 21, wherein the lymphocytes are at least one of CD8⁺T_{C} and CD8⁺T_{M}.

23. A method for treating COVID-19 disease in a patient having or suspected of having COVID-19 disease, the method comprising:
providing a composition including Mdivi-1; and
administering the composition to the patient.

24. The method according to claim 23, wherein the composition further includes a drug known as beneficial in treating COVID-19.

25. The method according to claim 24, wherein the drug known as beneficial in treating COVID-19 is at least one of cyclophilin, a cholesterol-lowering drug, a glucose-metabolism reducing drug, and an antioxidant drug.

26. A method for inhibiting viral replication of SARS-CoV-2 in cells of a patient having COVID-19 disease, the method comprising:
providing a composition including Mdivi-1; and
administering the composition to the patient.

27. A pharmaceutical composition for treatment of COVID-19 disease comprising a therapeutically effective dosage of Mdivi-1in at least one pharmaceutical carrier.

28. The pharmaceutical composition according to claim 27 for use in a method for inhibiting mitophagy in cells isolated from bronchoalveolar lavage fluid (BALF).

29. Use of the pharmaceutical composition according to claim 28, wherein the cells isolated from BALF are at least one of lymphocytes and epithelial cells (ECs).

30. Use of the pharmaceutical composition according to claim 28, wherein the bronchoalveolar lavage fluid (BALF) is obtained from at least one of a patient having or suspected of having COVID-19 disease, a healthy patient, and a patient having a non-COVID respiratory disease.

31. The pharmaceutical composition according to claim 27 for use in a method for restoring lymphocyte function in lymphocytes isolated from a patient having COVID-19 disease.

32. Use of the pharmaceutical composition according to claim 31, wherein the lymphocytes are at least one of CD8⁺T_{C} and CD8⁺T_{M}.

33. The pharmaceutical composition according to claim 27 for use in a method for restoring proliferation, activation, and memory formation of CD8⁺T_{C} and CD8⁺T_{M} isolated from a patient having COVID-19 disease.

34. The pharmaceutical composition according to claim 27 for use in a method for inhibiting mitophagy in lymphocytes of a patient having or suspected of having COVID-19 disease.

35. Use of the pharmaceutical composition according to claim 34, wherein the lymphocytes are at least one of CD8⁺T_{C} and CD8⁺T_{M}.

36. The pharmaceutical composition according to claim 27 for use in a method for treating COVID-19 disease in a patient having or suspected of having COVID-19 disease.

37. Use of the pharmaceutical composition according to claim 36, wherein the composition further includes a drug known as beneficial in treating COVID-19.

38. Use of the pharmaceutical composition according to claim 37, wherein the drug known as beneficial in treating COVID-19 is at least one of cyclophilin, a cholesterol-lowering drug, a glucose-metabolism reducing drug, and an antioxidant drug.

39. The pharmaceutical composition according to claim 27 for use in a method for method for inhibiting viral replication of SARS-CoV-2 in cells of a patient having COVID-19 disease.
